(19) United States
(12) Patent Application Publication
(10) Pub. No.: US 2016/0311817 A1
(43) Pub. Date: Oct. 27, 2016

(54) HETEROARYL SULTAM DERIVATIVES AS RORc MODULATORS

(71) Applicant: Genentech, Inc., South San Francisco, CA (US)

(72) Inventors: Benjamin Fauber, Austin, TX (US); Olivier Rene, San Francisco, CA (US); Sushant Malhotra, Burlingame, CA (US); Herbert Yajima, San Francisco, CA (US)

(73) Assignee: Genentech, Inc., South San Francisco, CA (US)

(21) Appl. No.: 15/202,911

(22) Filed: Jul. 6, 2016

Related U.S. Application Data
(63) Continuation of application No. PCT/EP2015/050290, filed on Jan. 9, 2015.
(60) Provisional application No. 61/925,831, filed on Jan. 10, 2014.

(51) Int. Cl. C07D 417/14; C07D 417/04

(52) U.S. Cl. CPC C07D 417/14; C07D 417/04

(57) ABSTRACT

Compounds of the formula I

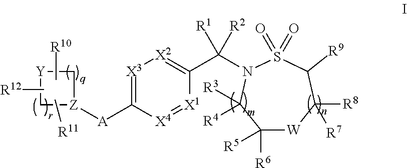

or a pharmaceutical salt thereof, wherein m, n, q, r, A, W, X¹, X², X³, X⁴, Y, Z, R¹, R², R³, R⁴, R⁵, R⁶, R⁷, R⁸, R⁹, R¹⁰ and R¹¹ are as defined herein. Also disclosed are methods of making the compounds and using the compounds for treatment of inflammatory diseases such as arthritis.

### RELATED APPLICATION DATA

This application is a Continuation of International Application No. PCT/EP2015/050290 filed on Jan. 9, 2015 which claims priority to Provisional Patent Application No. 61/925,831 filed on Jan. 10, 2014, the disclosures of which are incorporated by reference in their entirety.

### FIELD OF THE INVENTION

The invention pertains to compounds that modulate the function of retinoid-receptor related orphan receptor RORc (RORγ) and use of such compounds for treatment of autoimmune diseases.

### BACKGROUND OF THE INVENTION

T helper 17 cells (Th17) are interleukin (IL)-17 secreting CD4+ T cells involved in pathogenesis of autoimmune diseases such as rheumatoid arthritis, irritable bowel disease, psoriasis, psoriatic arthritis and spondyloarthridities. The retinoic acid-related orphan receptor γ (RORγ or RORc) is recognized as a transcription factor necessary for Th17 cell differentiation. RORc is an orphan member of the nuclear hormone receptor subfamily that includes RORα (RORa) and RORβ (RORb). RORc controls gene transcription by binding to DNA as a monomer. Selective modulation of RORc has been proposed as a route to discovery and development of Th17 cell-associated autoimmune diseases.

There is accordingly a need for compounds that inhibit RORc for use in treatment of autoimmune diseases such as rheumatoid arthritis, irritable bowel disease, psoriasis, psoriatic arthritis and spondyloarthridities.

### SUMMARY OF THE INVENTION

The invention provides compounds of formula I:

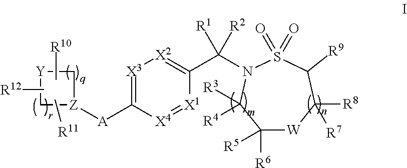

or a pharmaceutically acceptable salt thereof,
wherein:

m is 0 or 1;

n is 0 or 1;

q is 0, 1 or 2;

r is from 1 to 3;

A is: a bond; —(CRⱼRₖ)ₜ—; —C(O)—(CRⱼRₖ)ₜ—; —(CRⱼRₖ)ₜ—C(O)—; —NR^{a}—(CRⱼRₖ)ₜ—; —(CRⱼRₖ)ₜ—NR^{a}—; —C(O)NR^{a}—(CRⱼRₖ)ₜ—; —(CRⱼRₖ)ₜ—NR^{a}C(O)—; —O—(CRⱼRₖ)ₜ—; —(CRⱼRₖ)ₜ—O—; —S—(CRⱼRₖ)ₜ—; —(CRⱼRₖ)ₜ—S—; —SO₂—(CRⱼRₖ)ₜ—; or —(CRⱼRₖ)ₜ—SO₂—;

t is from 0 to 4;

W is: —CR^{b}R^{c}—; —O—; —S—; —SO₂—; or NR^{d}—;

one of X¹, X², X³ and X⁴ is N and the others are CR^{e}; or two of X¹, X², X³ and X⁴ are N and the others are CR^{e}; or three of X¹, X², X³ and X⁴ are N and the other is CR^{e}; or each of X¹, X², X³ and X⁴ is CR^{e};

Y is: —O—; —S—; SO₂—; —CR^{f}R^{g}—; or —NR^{h}—;

Z is: CH; or N;

R¹, R², R³, R⁴, R⁵, R⁶, R⁷ and R⁸ each independently is: hydrogen; or C₁₋₆alkyl which may be unsubstituted or substituted one or more times with halo;

or R³ and R⁴ together with the atom to which they are attached may form an ethylene group;

or R³ and R⁴ together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

or R⁵ and R⁶ together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

or R⁷ and R⁸ together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

or one of R³ and R⁴ together with one of R⁵ and R⁶ and the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

or one of R⁵ and R⁶ together with one of R⁷ and R⁸ and the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

R⁹ is: C₁₋₆alkyl; C₃-cycloalkyl; heterocyclyl; or heteroaryl; each of which may be unsubstituted or substituted one or more times with Rⁱ;

R¹⁰ is: hydrogen; carboxy; C₁₋₆alkyl-carbonyl; C₁₋₆alkoxy-carbonyl; oxo; hydroxy; aminocarbonyl; N—C₁₋₆alkyl-aminocarbonyl; N,N-di-C₁₋₆alkyl-aminocarbonyl; cyano; hydroxy-C₁₆alkyl; N—C₁₋₆alkoxy-C₁₋₆alkyl-aminocarbonyl; N-hydroxy-C₁₋₆alkyl-aminocarbonyl; N—C₁₋₆alkoxy-aminocarbonyl; halo; or C₁₋₆alkyl which may be unsubstituted or substituted one or more times with halo or oxo;

R¹¹ is: hydrogen; halo; carboxy; C₁₋₆alkyl-carbonyl; C₁₋₆alkoxy-carbonyl; oxo; hydroxy; aminocarbonyl; N—C₁₋₆alkyl-aminocarbonyl; N,N-di-C₁₋₆alkyl-aminocarbonyl; C₁₋₆alkyl-sulfonylamino; C₁₋₆alkyl-sulfonylamino-C₁₋₆alkyl; cyano; hydroxy-C₁₆alkyl; N—C₁₋₆alkoxy-C₁₋₆alkyl-aminocarbonyl; N-hydroxy-C₁₋₆alkyl-aminocarbonyl; N—C₁₋₆alkoxy-aminocarbonyl; or C₁₋₆alkyl which may be unsubstituted or substituted one or more times with halo or oxo;

R¹² is: hydrogen; halo; carboxy; C₁₋₆alkyl-carbonyl; C₁₋₆alkoxy-carbonyl; oxo; hydroxy; aminocarbonyl; N—C₁₋₆alkyl-aminocarbonyl; N,N-di-C₁₋₆alkyl-aminocarbonyl; cyano; hydroxy-C₁₆alkyl; N—C₁₋₆alkoxy-C₁₋₆alkyl-aminocarbonyl; N-hydroxy-C₁₋₆alkyl-aminocarbonyl; N—C₁₋₆alkoxy-aminocarbonyl; or C₁₋₆alkyl which may be unsubstituted or substituted one or more times with halo or oxo;

or R¹⁰ and R¹¹ together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

R^{a}, R^{b}, R^{c} and R^{d} each independent is: hydrogen; or C₁₋₆alkyl which may be unsubstituted or substituted one or more times with halo;

or R^{b} and R^{c} together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

or one of R^{b} and R^{c} together with one of R⁷ and R⁸ and the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

or one of R^{b} and R^{c} together with one of R⁵ and R⁶ and the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

each R^{e} is independently: hydrogen; C₁₋₆alkyl; halo; C₁₋₆alkoxy; or cyano; wherein the C₁₋₆alkyl moieties may be unsubstituted or substituted one or more times with halo;

R^{f} is: hydrogen; halo; or C₁₋₆alkyl which may be unsubstituted or substituted one or more times with halo;

R^{g} is: hydrogen; C₁₋₆alkyl; C₃₋₆cycloalkyl; C₃₋₆cycloalkenyl; C₃₋₆cycloalkyl-C₁₋₆alkyl; halo; C₁₋₆alkyl-carbonyl; C₃₋₆cycloalkyl-carbonyl; C₃₋₆cycloalkyl-C₁₋₆alkyl-carbonyl; cyano-C₁₋₆alkyl-carbonyl; hydroxy-C₁₋₆alkyl-carbonyl; C₁₋₆alkoxy-C₁₋₆alkyl-carbonyl; carboxy; N-cyano-aminocarbonyl; N-cyano-N—C₁₋₆alkyl-aminocarbonyl; N—C₁₋₆alkyl-acetimidamidyl; N,N′-di-C₁₋₆alkyl-acetimidamidyl; N′-cyano-N—C₁₋₆alkyl-acetimidamidyl; N′-hydroxy-acetimidamidyl; N′—C₁₋₆alkoxy-acetimidamidyl; N′-hydroxy-N—C₁₋₆alkyl-acetimidamidyl; N′—C₁₋₆alkoxy-N—C₁₋₆alkyl-acetimidamidyl; 2-nitro-1-N—C₁₋₆alkylamino-vinyl; formyl; C₁₋₆alkyl-sulfonyl; C₃₋₆cycloalkyl-sulfonyl; C₃₋₆cycloalkyl-C₁₋₆alkyl-sulfonyl; C₁₋₆alkyl-sulfonyl-C₁₋₆alkyl; aminocarbonyl; N-hydroxy-aminocarbonyl; N—C₁₋₆alkoxy-aminocarbonyl; N—C₁₋₆alkyl-aminocarbonyl; aminocarbonyl-C₁₋₆alkyl; N—C₁₋₆alkyl-aminocarbonyl-C₁₋₆alkyl; N,N-di-C₁₋₆alkyl-aminocarbonyl-C₁₋₆alkyl; C₁₋₆alkoxy-carbonyl; N-hydroxy-N—C₁₋₆alkyl-aminocarbonyl; N—C₁₋₆alkoxy-N—C₁₋₆alkyl-aminocarbonyl; N,N-di-C₁₋₆alkyl-aminocarbonyl; aminosulfonyl; N—C₁₋₆alkyl-aminosulfonyl; N,N-di-C₁₋₆alkyl-aminosulfonyl; cyano; C₁₋₆alkoxy; C₁₋₆alkyl-sulfonylamino; N—C₁₋₆alkyl-sulfonylaminocarbonyl; N—(C₁₋₆alkyl-sulfonyl)-N—C₁₋₆alkyl-aminocarbonyl; N—(C₁₋₆alkyl-sulfonyl)-amino-C₁₋₆alkyl; amino; N—C₁₋₆alkyl-amino; N,N-di-C₁₋₆alkyl-amino; halo-C₁₋₆alkyl; heterocyclyl; heteroaryl; or hydroxyl; wherein the C₁₋₆alkyl moieties may be unsubstituted or substituted one or more times with halo; and wherein the heterocyclyl, heteroaryl, C₃₋₆cycloalkyl, C₃₋₆cycloalkenyl and C₃₋₆cycloalkyl-C₁₋₆alkyl moieties may be unsubstituted or substituted one or more times with Rⁱ;

or R^{f} and R^{g} together with the atoms to which they are attached may form a four, five, six or seven membered saturated or partially saturated ring that may optionally include one or two heteroatoms selected from —O—, —NR^{a}— or —S—, and which may be optionally substituted one or more times with Rⁱ;

R^{h} is: hydrogen; C₁₋₆alkyl; C₃₋₆cycloalkyl; C₃₋₆cycloalkenyl; C₃₋₆cycloalkyl-C₁₋₆alkyl; C₁₋₆alkyl-carbonyl; C₃₋₆cycloalkyl-carbonyl; C₃₋₆cycloalkyl-C₁₋₆alkyl-carbonyl; cyano-C₁₋₆alkyl-carbonyl; hydroxy-C₁₋₆alkyl-carbonyl; C₁₋₆alkoxy-C₁₋₆alkyl-carbonyl; N-cyano-aminocarbonyl; N-cyano-N—C₁₋₆alkyl-aminocarbonyl; N—C₁₋₆alkyl-acetimidamidyl; N,N′-di-C₁₋₆alkyl-acetimidamidyl; N′-cyano-N—C₁₋₆alkyl-acetimidamidyl; N′-hydroxy-acetimidamidyl; N′—C₁₋₆alkoxy-acetimidamidyl; N′-hydroxy-N—C₁₋₆alkyl-acetimidamidyl; N′—C₁₋₆alkoxy-N—C₁₋₆alkyl-acetimidamidyl; 2-nitro-1-N—C₁₋₆alkylamino-vinyl; formyl; C₁₋₆alkyl-sulfonyl; C₃₋₆cycloalkyl-sulfonyl; C₃₋₆cycloalkyl-C₁₋₆alkyl-sulfonyl; C₁₋₆alkyl-sulfonyl-C₁₋₆alkyl; aminocarbonyl; N-hydroxy-aminocarbonyl; N—C₁₋₆alkoxy-aminocarbonyl; N—C₁₋₆alkyl-aminocarbonyl; N-hydroxy-N—C₁₋₆alkyl-aminocarbonyl; N—C₁₋₆alkoxy-N—C₁₋₆alkyl-aminocarbonyl; N,N-di-C₁₋₆alkyl-aminocarbonyl; aminosulfonyl; N—C₁₋₆alkyl-aminosulfonyl; N,N-di-C₁₋₆alkyl-aminosulfonyl; cyano; C₁₋₆alkyl-sulfonylamino; C₁₋₆alkyl-sulfonylamino-C₁₋₆alkyl; N—(C₁₋₆alkyl-sulfonyl)aminocarbonyl; N—(C₁₋₆alkyl-sulfonyl)-N—C₁₋₆alkyl-aminocarbonyl; N—(C₁₋₆alkyl-sulfonyl)-amino-C₁₋₆alkyl; aminocarbonyl-C₁₋₆alkyl; N—C₁₋₆alkyl-aminocarbonyl-C₁₋₆alkyl; N,N-di-C₁₋₆alkyl-aminocarbonyl-C₁₋₆alkyl; C₁₋₆alkoxy-carbonyl; halo-C₁₋₆alkyl; heterocyclyl; or heteroaryl; wherein the C₁₋₆alkyl moieties may be unsubstituted or substituted one or more times with halo; and wherein the heterocyclyl, heteroaryl, C₃₋₆cycloalkyl, C₃₋₆cycloalkenyl and C₃₋₆cycloalkyl-C₁₋₆alkyl moieties may be unsubstituted or substituted one or more times with Rⁱ;

or R^{h} and one of R¹⁰ and R¹¹ together with the atoms to which they are attached may form a four, five, six or seven membered aromatic, partially saturated or unsaturated ring that may optionally include one or two additional heteroatom selected from —O—, —NR^{a}— or —S—, and which may be optionally substituted one or more times with Rⁱ—;

or one of R^{f} and R^{g} and one of R¹⁰ and R¹¹ together with the atoms to which they are attached may form a three, four, five, six or seven membered aromatic, partially saturated or unsaturated ring that may optionally include an additional heteroatom selected from —O—, —NR^{a}— or —S—, and which may be optionally substituted one or more times with Rⁱ;

Rⁱ is: C₁₋₆alkyl; halo; oxo; hydroxy; acetyl; C₁₋₆alkyl-carbonyl; amino-carbonyl; hydroxy-C₁₋₆alkyl; cyano; heteroaryl; or C₁₋₆alkoxy; wherein the C₁₋₆alkyl moieties may be unsubstituted or substituted one or more times with halo; and

R^{j} and R^{h} each independent is: hydrogen; or C₁₋₆alkyl which may be unsubstituted or substituted one or more times with halo.

The invention also provides and pharmaceutical compositions comprising the compounds, methods of using the compounds, and methods of preparing the compounds.

### DETAILED DESCRIPTION OF THE INVENTION

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

“Alkyl” means the monovalent linear or branched saturated hydrocarbon moiety, consisting solely of carbon and hydrogen atoms, having from one to twelve carbon atoms. “Lower alkyl” refers to an alkyl group of one to six carbon atoms, i.e. C₁-C₆alkyl. Examples of alkyl groups include, but are not limited to, methyl, ethyl, propyl, isopropyl, isobutyl, sec-butyl, tert-butyl, pentyl, n-hexyl, octyl, dodecyl, and the like.

“Alkenyl” means a linear monovalent hydrocarbon radical of two to six carbon atoms or a branched monovalent hydrocarbon radical of three to six carbon atoms, containing at least one double bond, e.g., ethenyl, propenyl, and the like.

“Alkynyl” means a linear monovalent hydrocarbon radical of two to six carbon atoms or a branched monovalent hydrocarbon radical of three to six carbon atoms, containing at least one triple bond, e.g., ethynyl, propynyl, and the like.

“Alkylene” means a linear saturated divalent hydrocarbon radical of one to six carbon atoms or a branched saturated divalent hydrocarbon radical of three to six carbon atoms, e.g., methylene, ethylene, 2,2-dimethylethylene, propylene, 2-methylpropylene, butylene, pentylene, and the like.

“Alkoxy” and “alkyloxy”, which may be used interchangeably, mean a moiety of the formula —OR, wherein R is an alkyl moiety as defined herein. Examples of alkoxy moieties include, but are not limited to, methoxy, ethoxy, isopropoxy, and the like.

“Alkoxyalkyl” means a moiety of the formula R^{a}—O—R^{b}—, where R^{a} is alkyl and R^{b} is alkylene as defined herein. Exemplary alkoxyalkyl groups include, by way of example, 2-methoxyethyl, 3-methoxypropyl, 1-methyl-2-methoxyethyl, 1-(2-methoxyethyl)-3-methoxypropyl, and 1-(2-methoxyethyl)-3-methoxypropyl.

“Alkoxyalkoxy” means a group of the formula —O—R—R′ wherein R is alkylene and R′ is alkoxy as defined herein.

“Alkylcarbonyl” means a moiety of the formula —C(O)—R, wherein R is alkyl as defined herein.

“Alkoxycarbonyl” means a group of the formula —C(O)—R wherein R is alkoxy as defined herein.

“Alkylcarbonylalkyl” means a group of the formula —R—C(O)—R wherein R is alkylene and R′ is alkyl as defined herein.

“Alkoxyalkylcarbonyl” means a moiety of the formula —C(O)—R—R′, wherein R is alkylene and R′ is alkoxy as defined herein.

“Alkoxycarbonylalkyl” means a group of the formula —R—C(O)—R wherein R is alkylene and R′ is alkoxy as defined herein.

“Alkoxycarbonylalkoxy” means a group of the formula —O—R—C(O)—R′ wherein R is alkylene and R′ is alkoxy as defined herein.

“Hydroxycarbonylalkoxy” means a group of the formula —O—R—C(O)—OH wherein R is alkylene as defined herein.

“Alkylaminocarbonylalkoxy” means a group of the formula —O—R—C(O)—NHR′ wherein R is alkylene and R′ is alkyl as defined herein.

“Dialkylaminocarbonylalkoxy” means a group of the formula —O—R—C(O)—NR′R″ wherein R is alkylene and R′ and R″ are alkyl as defined herein.

“Alkylaminoalkoxy” means a group of the formula —O—R—NHR′ wherein R is alkylene and R′ is alkyl as defined herein.

“Dialkylaminoalkoxy” means a group of the formula —O—R—NR′R′ wherein R is alkylene and R′ and R″ are alkyl as defined herein.

“Alkylsulfonyl” means a moiety of the formula —SO₂R, wherein R is alkyl as defined herein.

“Alkylsulfonylalkyl means a moiety of the formula —R′—SO₂—R” where R′ is alkylene and R″ is alkyl as defined herein.

“Alkylsulfonylalkoxy” means a group of the formula —O—R—SO₂—R′ wherein R is alkylene and R′ is alkyl as defined herein.

“Amino means a moiety of the formula —NRR′ wherein R and R′ each independently is hydrogen or alkyl as defined herein. “Amino thus includes “alkylamino (where one of R and R′ is alkyl and the other is hydrogen) and “dialkylamino (where R and R′ are both alkyl.

“Aminocarbonyl” means a group of the formula —C(O)—R wherein R is amino as defined herein.

“N-hydroxy-aminocarbonyl” means a group of the formula —C(O)—NR—OH wherein R is hydrogen or alkyl as defined herein.

“N-alkoxy-aminocarbonyl” means a group of the formula —C(O)—NR—R′ wherein R is hydrogen or alkyl and R′ is alkoxy as defined herein.

“N-alkyl-aminocarbonyl means a group of the formula —C(O)—NH—R wherein R is alkyl as defined herein.

“N-hydroxy-N-alkylaminocarbonyl means a group of the formula —C(O)—NRR′ wherein R is alkyl as defined herein and R′ is hydroxy.

“N-alkoxy-N-alkylaminocarbonyl” means a group of the formula —C(O)—NRR′ wherein R is alkyl and R′ is alkoxy as defined herein.

“N,N-di-C₁₋₆alkyl-aminocarbonyl” means a group of the formula —C(O)—NRR′ wherein R and R′ are alkyl as defined herein.

“Aminosulfonyl” means a group of the formula —SO₂—NH₂.

“N-alkylaminosulfonyl” means a group of the formula —SO₂—NHR wherein R is alkyl as defined herein.

“N,N-dialkylaminosulfonyl” means a group of the formula —SO₂—NRR′ wherein R and R′ are alkyl as defined herein.

“Alkylsulfonylamino” means a group of the formula —NR′—SO₂—R wherein Rid alkyl and R′ is hydrogen or alkyl as defined herein.

“N-(alkylsulfonyl)-aminoalkyl” means a group of the formula —R—NH—SO₂—R′ wherein R is alkylene and R′ is alkyl as defined herein.

“N-(Alkylsulfonyl)aminocarbonyl” means a group of the formula —C(O)—NH—SO₂—R wherein wherein R is alkyl as defined herein.

“N-(Alkylsulfonyl)-N-alkylaminocarbonyl” means a group of the formula —C(O)—NR—SO₂—R′ wherein R and R′ are alkyl as defined herein.

“N-Alkoxyalkyl-aminocarbonyl” means a group of the formula —C(O)—NR—R′—OR″ wherein R is hydrogen or alkyl, R′ is alkylene, and R″ is alkyl as defined herein.

“N-Hydroxyalkyl-aminocarbonyl” means a group of the formula —C(O)—NR—R′—OH″ wherein R is hydrogen or alkyl and R′ is alkylene as defined herein.

“Alkoxyamino” means a moiety of the formula —NR—OR′ wherein R is hydrogen or alkyl and R′ is alkyl as defined herein.

“Alkylsulfanyl” means a moiety of the formula —SR wherein R is alkyl as defined herein.

“Aminoalkyl” means a group —R—R′ wherein R′ is amino and R is alkylene as defined herein.

“Aminoalkyl” includes aminomethyl, aminoethyl, 1-aminopropyl, 2-aminopropyl, and the like. The amino moiety of “aminoalkyl” may be substituted once or twice with alkyl to provide “alkylaminoalkyl” and “dialkylaminoalkyl” respectively. “Alkylaminoalkyl” includes methylaminomethyl, methylaminoethyl, methylaminopropyl, ethylaminoethyl and the like.

“Dialkylaminoalkyl” includes dimethylaminomethyl, dimethylaminoethyl, dimethylaminopropyl, N-methyl-N-ethylaminoethyl, and the like.

“Aminoalkoxy” means a group —OR—R′ wherein R′ is amino and R is alkylene as defined herein.

“Alkylsulfonylamido” means a moiety of the formula —NR′SO₂—R wherein R is alkyl and R′ is hydrogen or alkyl.

“Aminocarbonyloxyalkyl” or “carbamylalkyl” means a group of the formula —R—O—C(O)—NR′R″ wherein R is alkylene and R′, R″ each independently is hydrogen or alkyl as defined herein.

“Alkynylalkoxy” means a group of the formula —O—R—R′ wherein R is alkylene and R′ is alkynyl as defined herein.

“Aryl” means a monovalent cyclic aromatic hydrocarbon moiety consisting of a mono-, bi- or tricyclic aromatic ring. The aryl group can be optionally substituted as defined herein. Examples of aryl moieties include, but are not limited to, phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like, of which may be optionally substituted as defined herein.

“Arylalkyl” and “Aralkyl”, which may be used interchangeably, mean a radical-R^{a}R^{b} where R^{a} is an alkylene group and R^{b} is an aryl group as defined herein; e.g., phenylalkyls such as benzyl, phenylethyl, 3-(3-chlorophenyl)-2-methylpentyl, and the like are examples of arylalkyl.

“Arylsulfonyl means a group of the formula —SO₂—R wherein R is aryl as defined herein.

“Aryloxy” means a group of the formula —O—R wherein R is aryl as defined herein.

“Aralkyloxy” means a group of the formula —O—R—R″ wherein R is alkylene and R′ is aryl as defined herein.

“Carboxy” or “hydroxycarbonyl”, which may be used interchangeably, means a group of the formula —C(O)—OH.

“Cyanoalkyl” “means a moiety of the formula —R′—R″, where R′ is alkylene as defined herein and R″ is cyano or nitrile.

“Cycloalkyl” means a monovalent saturated carbocyclic moiety consisting of mono- or bicyclic rings. Particular cycloalkyl are unsubstituted or substituted with alkyl. Cycloalkyl can optionally be substituted as defined herein. Unless defined otherwise, cycloalkyl may be optionally substituted with one or more substituents, wherein each substituent is independently hydroxy, alkyl, alkoxy, halo, haloalkyl, amino, monoalkylamino, or dialkylamino. Examples of cycloalkyl moieties include, but are not limited to, cyclopropyl, cyclobutyl, cyclopentyl, cyclohexyl, cycloheptyl, and the like, including partially unsaturated (cycloalkenyl) derivatives thereof.

“Cycloalkenyl” means a cycloalkyl as defined herein that includes at least one double bond or unsaturation. Exemplary cycloalkenyl include cyclohexenyl, cyclopentenyl, cyclobutenyl and the like.

“Cycloalkylalkyl” means a moiety of the formula —R′—R″, where R′ is alkylene and R″ is cycloalkyl as defined herein.

“Cycloalkylalkoxy” means a group of the formula —O—R—R′ wherein R is alkylene and R′ is cycloalkyl as defined herein.

“Cycloalkylcarbonyl” means a moiety of the formula —C(O)—R, wherein R is cycloalkyl as defined herein.

“C₃₋₆cycloalkyl-C₁₋₆alkyl-carbonyl” means a moiety of the formula —C(O)—R, wherein R is cycloalkylalkyl as defined herein.

“Cyanoalkylcarbonyl” means a moiety of the formula —C(O)—R—R′, wherein R is alkylene as defined herein and R′ is cyano or nitrile.

“N-Cyano-aminocarbonyl” means a moiety of the formula —C(O)—NHR, wherein R is cyano or nitrile.

“N-Cyano-N-alkyl-aminocarbonyl” means a moiety of the formula —C(O)—NRR′—R, wherein R′ is alkyl as defined herein and R is cyano or nitrile.

“Cycloalkylsulfonyl” means a group of the formula —SO₂—R wherein R is cycloalkyl as defined herein.

“Cycloalkylalkylsulfonyl” means a group of the formula —SO₂—R wherein R is cycloalkylalkyl as defined herein.

“Formyl” means a moiety of the formula —C(O)—H.

“Heteroaryl” means a monocyclic or bicyclic radical of 5 to 12 ring atoms having at least one aromatic ring containing one, two, or three ring heteroatoms selected from N, O, or S, the remaining ring atoms being C, with the understanding that the attachment point of the heteroaryl radical will be on an aromatic ring. The heteroaryl ring may be optionally substituted as defined herein. Examples of heteroaryl moieties include, but are not limited to, optionally substituted imidazolyl, oxazolyl, isoxazolyl, thiazolyl, isothiazolyl, oxadiazolyl, thiadiazolyl, pyrazinyl, thienyl, benzothienyl, thiophenyl, furanyl, pyranyl, pyridyl, pyrrolyl, pyrazolyl, pyrimidyl, quinolinyl, isoquinolinyl, benzofuryl, benzothiophenyl, benzothiopyranyl, benzimidazolyl, benzooxazolyl, benzooxadiazolyl, benzothiazolyl, benzothiadiazolyl, benzopyranyl, indolyl, isoindolyl, triazolyl, triazinyl, quinoxalinyl, purinyl, quinazolinyl, quinolizinyl, naphthyridinyl, pteridinyl, carbazolyl, azepinyl, diazepinyl, acridinyl and the like, each of which may be optionally substituted as defined herein.

Heteroarylalkyl” or “heteroaralkyl” means a group of the formula —R—R′ wherein R is alkylene and R′ is heteroaryl as defined herein.

“Heteroarylsulfonyl means a group of the formula —SO₂—R wherein R is heteroaryl as defined herein.

“Heteroaryloxy” means a group of the formula —O—R wherein R is heteroaryl as defined herein.

“Heteroaralkyloxy” means a group of the formula —O—R—R″ wherein R is alkylene and R′ is heteroaryl as defined herein.

The terms “halo”, “halogen” and “halide”, which may be used interchangeably, refer to a substituent fluoro, chloro, bromo, or iodo.

“Haloalkyl” means alkyl as defined herein in which one or more hydrogen has been replaced with same or different halogen. Exemplary haloalkyls include —CH₂Cl, —CH₂CF₃, —CH₂CCl₃, perfluoroalkyl (e.g., —CF₃), and the like.

“Haloalkoxy” means a moiety of the formula —OR, wherein R is a haloalkyl moiety as defined herein. An exemplary haloalkoxy is difluoromethoxy.

“Heterocycloamino” means a saturated ring wherein at least one ring atom is N, NH or N-alkyl and the remaining ring atoms form an alkylene group.

“Heterocyclyl” means a monovalent saturated moiety, consisting of one to three rings, incorporating one, two, or three or four heteroatoms (chosen from nitrogen, oxygen or sulfur). The heterocyclyl ring may be optionally substituted as defined herein. Examples of heterocyclyl moieties include, but are not limited to, optionally substituted piperidinyl, piperazinyl, morpholinyl, thiomorpholinyl, azepinyl, pyrrolidinyl, azetidinyl, tetrahydropyranyl, tetrahydrofuranyl, oxetanyl and the like. Such heterocyclyl may be optionally substituted as defined herein.

“Heterocyclylalkyl” means a moiety of the formula —R—R′ wherein R is alkylene and R′ is heterocyclyl as defined herein.

“Heterocyclyloxy” means a moiety of the formula —OR wherein R is heterocyclyl as defined herein.

“Heterocyclylalkoxy” means a moiety of the formula —OR—R′ wherein R is alkylene and R′ is heterocyclyl as defined herein.

“Hydroxyalkoxy” means a moiety of the formula —OR wherein R is hydroxyalkyl as defined herein.

“Hydroxyalkylamino” means a moiety of the formula —NR—R′ wherein R is hydrogen or alkyl and R′ is hydroxyalkyl as defined herein.

“Hydroxyalkylaminoalkyl” means a moiety of the formula —R—NR′—R″ wherein R is alkylene, R′ is hydrogen or alkyl, and R″ is hydroxyalkyl as defined herein.

“Hydroxycarbonylalkyl” or “carboxyalkyl” means a group of the formula —R—(CO)—OH where R is alkylene as defined herein.

“Hydroxycarbonylalkoxy” means a group of the formula —O—R—C(O)—OH wherein R is alkylene as defined herein.

“Hydroxyalkylcarbonyl” means a moiety of the formula —C(O)—R—R′, wherein R is alkylene as defined herein and R′ is hydroxy.

“Hydroxyalkyloxycarbonylalkyl” or “hydroxyalkoxycarbonylalkyl” means a group of the formula —R—C(O)—O—R—OH wherein each R is alkylene and may be the same or different.

“Hydroxyalkyl” means an alkyl moiety as defined herein, substituted with one or more, for example, one, two or three hydroxy groups, provided that the same carbon atom does not carry more than one hydroxy group. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl and 2-(hydroxymethyl)-3-hydroxypropyl

“Hydroxycycloalkyl” means a cycloalkyl moiety as defined herein wherein one, two or three hydrogen atoms in the cycloalkyl radical have been replaced with a hydroxy substituent. Representative examples include, but are not limited to, 2-, 3-, or 4-hydroxycyclohexyl, and the like.

“Oxo” means a group of the formula ═O (i.e., an oxygen with a double bond). Thus, for example, a 1-oxo-ethyl group is an acetyl group.

“Alkoxy hydroxyalkyl” and “hydroxy alkoxyalkyl”, which may be used interchangeably, means an alkyl as defined herein that is substituted at least once with hydroxy and at least once with alkoxy.

“Alkoxy hydroxyalkyl” and “hydroxy alkoxyalkyl” thus encompass, for example, 2-hydroxy-3-methoxy-propan-1-yl and the like.

“Urea” or “ureido” means a group of the formula —NR′—C(O)—NR″R′″ wherein R′, R″ and R′″ each independently is hydrogen or alkyl.

“Carbamate” means a group of the formula —O—C(O)—NR′R″ wherein R′ and R″ each independently is hydrogen or alkyl.

“Carboxy” means a group of the formula —O—C(O)—OH.

“Sulfonamido” means a group of the formula —SO₂—NR′R″ wherein R′, R″ and R′″ each independently is hydrogen or alkyl.

“Optionally substituted” when used in association with an “aryl”, phenyl”, “heteroaryl” “cycloalkyl” or “heterocyclyl” moiety means that such moiety may be unsubstituted (i.e., all open valencies are occupied by a hydrogen atom) or substituted with specific groups as related herein.

“Leaving group” means the group with the meaning conventionally associated with it in synthetic organic chemistry, i.e., an atom or group displaceable under substitution reaction conditions. Examples of leaving groups include, but are not limited to, halogen, alkane- or arylenesulfonyloxy, such as methanesulfonyloxy, ethanesulfonyloxy, thiomethyl, benzenesulfonyloxy, tosyloxy, and thienyloxy, dihalophosphinoyloxy, optionally substituted benzyloxy, isopropyloxy, acyloxy, and the like.

“Modulator” means a molecule that interacts with a target. The interactions include, but are not limited to, agonist, antagonist, and the like, as defined herein.

“Optional” or “optionally” means that the subsequently described event or circumstance may but need not occur, and that the description includes instances where the event or circumstance occurs and instances in which it does not.

“Disease” and “Disease state” means any disease, condition, symptom, disorder or indication.

“Inert organic solvent” or “inert solvent” means the solvent is inert under the conditions of the reaction being described in conjunction therewith, including for example, benzene, toluene, acetonitrile, tetrahydrofuran, N,N-dimethylformamide, chloroform, methylene chloride or dichloromethane, dichloroethane, diethyl ether, ethyl acetate, acetone, methyl ethyl ketone, methanol, ethanol, propanol, isopropanol, tert-butanol, dioxane, pyridine, and the like. Unless specified to the contrary, the solvents used in the reactions of the present invention are inert solvents.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

“Pharmaceutically acceptable salts” of a compound means salts that are pharmaceutically acceptable, as defined herein, and that possess the desired pharmacological activity of the parent compound.

It should be understood that all references to pharmaceutically acceptable salts include solvent addition forms (solvates) or crystal forms (polymorphs) as defined herein, of the same acid addition salt.

“Protective group” or “protecting group” means the group which selectively blocks one reactive site in a multifunctional compound such that a chemical reaction can be carried out selectively at another unprotected reactive site in the meaning conventionally associated with it in synthetic chemistry. Certain processes of this invention rely upon the protective groups to block reactive nitrogen and/or oxygen atoms present in the reactants. For example, the terms “amino-protecting group” and “nitrogen protecting group” are used interchangeably herein and refer to those organic groups intended to protect the nitrogen atom against undesirable reactions during synthetic procedures. Exemplary nitrogen protecting groups include, but are not limited to, trifluoroacetyl, acetamido, benzyl (Bn), benzyloxycarbonyl (carbobenzyloxy, CBZ), p-methoxybenzyloxycarbonyl, p-nitrobenzyloxycarbonyl, tert-butoxycarbonyl (BOC), and the like. The artisan in the art will know how to chose a group for the ease of removal and for the ability to withstand the following reactions.

“Solvates” means solvent additions forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

“Arthritis” means a disease or condition that causes damage to joints of the body and pain associated with such joint damage. Arthritis includes rheumatoid arthritis, osteoarthritis, psoriatic arthritis, septic arthritis, spondyloarthropathies, gouty arthritis, systemic lupus erythematosus and juvenile arthritis, osteoarthritis, and other arthritic conditions.

“Respiratory disorder” refers to, without limitation, chronic obstructive pulmonary disease (COPD), asthma, bronchospasm, and the like.

“Subject” means mammals and non-mammals. Mammals means any member of the mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The terms “those defined above” and “those defined herein” when referring to a variable incorporates by reference the broad definition of the variable as well as particular definitions, if any.

“Treating” or “treatment” of a disease state includes, inter alia, inhibiting the disease state, i.e., arresting the development of the disease state or its clinical symptoms, and/or relieving the disease state, i.e., causing temporary or permanent regression of the disease state or its clinical symptoms.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

Nomenclature and Structures

In general, the nomenclature and chemical names used in this application are based on ChembioOffice™ by Cambridge Soft™. Any open valency appearing on a carbon, oxygen sulfur or nitrogen atom in the structures herein indicates the presence of a hydrogen atom unless indicated otherwise. Where a nitrogen-containing heteroaryl ring is shown with an open valency on a nitrogen atom, and variables such as R^{a}, R^{b} or R^{c} are shown on the heteroaryl ring, such variables may be bound or joined to the open valency nitrogen. Where a chiral center exists in a structure but no specific stereochemistry is shown for the chiral center, both enantiomers associated with the chiral center are encompassed by the structure. Where a structure shown herein may exist in multiple tautomeric forms, all such tautomers are encompassed by the structure. The atoms represented in the structures herein are intended to encompass all naturally occurring isotopes of such atoms. Thus, for example, the hydrogen atoms represented herein are meant to include deuterium and tritium, and the carbon atoms are meant to include C¹³ and C¹⁴ isotopes. One or more carbon atom(s) of a compound of the invention may be replaced by a silicon atom(s), and it is contemplated that one or more oxygen atom(s) of a compound of the invention may be replaced by a sulfur or selenium atom(s).

Compounds of the Invention

The invention provides compounds of formula I:

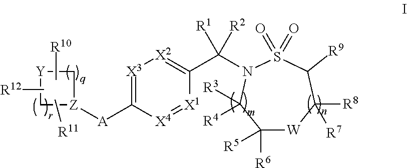

or a pharmaceutically acceptable salt thereof,
wherein:

m is 0 or 1;

n is 0 or 1;

q is 0, 1 or 2;

r is from 1 to 3;

A is: a bond; —(CRⱼRₖ)ₜ—; —C(O)—(CRⱼRₖ)ₜ—; —(CRⱼRₖ)ₜ—C(O)—; —NR^{a}—(CRⱼRₖ)ₜ—; —(CRⱼRₖ)ₜ—NR^{a}—; —C(O)NR^{a}—(CRⱼRₖ)ₜ—; —(CRⱼRₖ)ₜ—NR^{a}C(O)—; —O—(CRⱼRₖ)ₜ—; —(CRⱼRₖ)ₜ—O—; —S—(CRⱼRₖ)ₜ—; —(CRⱼRₖ)ₜ—S—; —SO₂—(CRⱼRₖ)ₜ—; or —(CRⱼRₖ)ₜ—SO₂—;

t is from 0 to 4;

W is: —CR^{b}R^{c}—; —O—; —S—; —SO₂—; or NR^{d}—;

one of X¹, X², X³ and X⁴ is N and the others are CR^{e}; or two of X¹, X², X³ and X⁴ are N and the others are CR^{e}; or three of X¹, X², X³ and X⁴ are N and the other is CR^{e}; or each of X¹, X², X³ and X⁴ is CR^{e};

Y is: —O—; —S—; SO₂—; —CR^{f}R^{g}—; or NR^{h}—;

Z is: CH; or N;

R¹, R², R³, R⁴, R⁵, R⁶, R⁷ and R⁸ each independently is: hydrogen; or C₁₋₆alkyl which may be unsubstituted or substituted one or more times with halo;

or R³ and R⁴ together with the atom to which they are attached may form an ethylene group;

or R³ and R⁴ together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

or R⁵ and R⁶ together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

or R⁷ and R⁸ together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

or one of R³ and R⁴ together with one of R⁵ and R⁶ and the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

or one of R⁵ and R⁶ together with one of R⁷ and R⁸ and the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

R⁹ is: C₁₋₆alkyl; C₃-cycloalkyl; heterocyclyl; or heteroaryl; each of which may be unsubstituted or substituted one or more times with Rⁱ;

R¹⁰ is: hydrogen; carboxy; C₁₋₆alkyl-carbonyl; C₁₋₆alkoxy-carbonyl; oxo; hydroxy; aminocarbonyl; N—C₁₋₆alkyl-aminocarbonyl; N,N-di-C₁₋₆alkyl-aminocarbonyl; cyano; hydroxy-C₁₆alkyl; N—C₁₋₆alkoxy-C₁₋₆alkyl-aminocarbonyl; N-hydroxy-C₁₋₆alkyl-aminocarbonyl; N—C₁₋₆alkoxy-aminocarbonyl; halo; or C₁₋₆alkyl which may be unsubstituted or substituted one or more times with halo or oxo;

R¹¹ is: hydrogen; halo; carboxy; C₁₋₆alkyl-carbonyl; C₁₋₆alkoxy-carbonyl; oxo; hydroxy; aminocarbonyl; N—C₁₋₆alkyl-aminocarbonyl; N,N-di-C₁₋₆alkyl-aminocarbonyl; C₁₋₆alkyl-sulfonylamino; C₁₋₆alkyl-sulfonylamino-C₁₋₆alkyl; cyano; hydroxy-C₁₆alkyl; N—C₁₋₆alkoxy-C₁₋₆alkyl-aminocarbonyl; N-hydroxy-C₁₋₆alkyl-aminocarbonyl; N—C₁₋₆alkoxy-aminocarbonyl; or C₁₋₆alkyl which may be unsubstituted or substituted one or more times with halo or oxo;

R¹² is: hydrogen; halo; carboxy; C₁₋₆alkyl-carbonyl; C₁₋₆alkoxy-carbonyl; oxo; hydroxy; aminocarbonyl; N—C₁₋₆alkyl-aminocarbonyl; N,N-di-C₁₋₆alkyl-aminocarbonyl; cyano; hydroxy-C₁₆alkyl; N—C₁₋₆alkoxy-C₁₋₆alkyl-aminocarbonyl; N-hydroxy-C₁₋₆alkyl-aminocarbonyl; N—C₁₋₆alkoxy-aminocarbonyl; or C₁₋₆alkyl which may be unsubstituted or substituted one or more times with halo or oxo;

or R¹⁰ and R¹¹ together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

R^{a}, R^{b}, R^{c} and R^{d} each independent is: hydrogen; or C₁₋₆alkyl which may be unsubstituted or substituted one or more times with halo;

or R^{b} and R^{c} together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

or one of R^{b} and R^{c} together with one of R⁷ and R⁸ and the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

or one of R^{b} and R^{c} together with one of R⁵ and R⁶ and the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;

each R^{e} is independently: hydrogen; C₁₋₆alkyl; halo; C₁₋₆alkoxy; or cyano; wherein the C₁₋₆alkyl moieties may be unsubstituted or substituted one or more times with halo;

R^{f} is: hydrogen; halo; or C₁₋₆alkyl which may be unsubstituted or substituted one or more times with halo;

R^{g} is: hydrogen; C₁₋₆alkyl; C₃₋₆cycloalkyl; C₃₋₆cycloalkenyl; C₃₋₆cycloalkyl-C₁₋₆alkyl; halo; C₁₋₆alkyl-carbonyl; C₃₋₆cycloalkyl-carbonyl; C₃₋₆cycloalkyl-C₁₋₆alkyl-carbonyl; cyano-C₁₋₆alkyl-carbonyl; hydroxy-C₁₋₆alkyl-carbonyl; C₁₋₆alkoxy-C₁₋₆alkyl-carbonyl; carboxy; N-cyano-aminocarbonyl; N-cyano-N—C₁₋₆alkyl-aminocarbonyl; N—C₁₋₆alkyl-acetimidamidyl; N,N′-di-C₁₋₆alkyl-acetimidamidyl; N′-cyano-N—C₁₋₆alkyl-acetimidamidyl; N′-hydroxy-acetimidamidyl; N′—C₁₋₆alkoxy-acetimidamidyl; N′-hydroxy-N—C₁₋₆alkyl-acetimidamidyl; N′—C₁₋₆alkoxy-N—C₁₋₆alkyl-acetimidamidyl; 2-nitro-1-N—C₁₋₆alkylamino-vinyl; formyl; C₁₋₆alkyl-sulfonyl; C₃₋₆cycloalkyl-sulfonyl; C₃₋₆cycloalkyl-C₁₋₆alkyl-sulfonyl; C₁₋₆alkyl-sulfonyl-C₁₋₆alkyl; aminocarbonyl; N-hydroxy-aminocarbonyl; N—C₁₋₆alkoxy-aminocarbonyl; N—C₁₋₆alkyl-aminocarbonyl; aminocarbonyl-C₁₋₆alkyl; N—C₁₋₆alkyl-aminocarbonyl-C₁₋₆alkyl; N,N-di-C₁₋₆alkyl-aminocarbonyl-C₁₋₆alkyl; C₁₋₆alkoxy-carbonyl; N-hydroxy-N—C₁₋₆alkyl-aminocarbonyl; N—C₁₋₆alkoxy-N—C₁₋₆alkyl-aminocarbonyl; N,N-di-C₁₋₆alkyl-aminocarbonyl; aminosulfonyl; N—C₁₋₆alkyl-aminosulfonyl; N,N-di-C₁₋₆alkyl-aminosulfonyl; cyano; C₁₋₆alkoxy; C₁₋₆alkyl-sulfonylamino; N—C₁₋₆alkyl-sulfonylaminocarbonyl; N—(C₁₋₆alkyl-sulfonyl)-N—C₁₋₆alkyl-aminocarbonyl; N—(C₁₋₆alkyl-sulfonyl)-amino-C₁₋₆alkyl; amino; N—C₁₋₆alkyl-amino; N,N-di-C₁₋₆alkyl-amino; halo-C₁₋₆alkyl; heterocyclyl; heteroaryl; or hydroxyl; wherein the C₁₋₆alkyl moieties may be unsubstituted or substituted one or more times with halo; and wherein the heterocyclyl, heteroaryl, C₃₋₆cycloalkyl, C₃₋₆cycloalkenyl and C₃₋₆cycloalkyl-C₁₋₆alkyl moieties may be unsubstituted or substituted one or more times with Rⁱ;

or R^{f} and R^{g} together with the atoms to which they are attached may form a four, five, six or seven membered saturated or partially saturated ring that may optionally include one or two heteroatoms selected from —O—, —NR^{a}— or —S—, and which may be optionally substituted one or more times with Rⁱ;

R^{h} is: hydrogen; C₁₋₆alkyl; C₃₋₆cycloalkyl; C₃₋₆cycloalkenyl; C₃₋₆cycloalkyl-C₁₋₆alkyl; C₁₋₆alkyl-carbonyl; C₃₋₆cycloalkyl-carbonyl; C₃₋₆cycloalkyl-C₁₋₆alkyl-carbonyl; cyano-C₁₋₆alkyl-carbonyl; hydroxy-C₁₋₆alkyl-carbonyl; C₁₋₆alkoxy-C₁₋₆alkyl-carbonyl; N-cyano-aminocarbonyl; N-cyano-N—C₁₋₆alkyl-aminocarbonyl; N—C₁₋₆alkyl-acetimidamidyl; N,N′-di-C₁₋₆alkyl-acetimidamidyl; N′-cyano-N—C₁₋₆alkyl-acetimidamidyl; N′-hydroxy-acetimidamidyl; N′—C₁₋₆alkoxy-acetimidamidyl; N′-hydroxy-N—C₁₋₆alkyl-acetimidamidyl; N′—C₁₋₆alkoxy-N—C₁₋₆alkyl-acetimidamidyl; 2-nitro-1-N—C₁₋₆alkylamino-vinyl; formyl; C₁₋₆alkyl-sulfonyl; C₃₋₆cycloalkyl-sulfonyl; C₃₋₆cycloalkyl-C₁₋₆alkyl-sulfonyl; C₁₋₆alkyl-sulfonyl-C₁₋₆alkyl; aminocarbonyl; N-hydroxy-aminocarbonyl; N—C₁₋₆alkoxy-aminocarbonyl; N—C₁₋₆alkyl-aminocarbonyl; N-hydroxy-N—C₁₋₆alkyl-aminocarbonyl; N—C₁₋₆alkoxy-N—C₁₋₆alkyl-aminocarbonyl; N,N-di-C₁₋₆alkyl-aminocarbonyl; aminosulfonyl; N—C₁₋₆alkyl-aminosulfonyl; N,N-di-C₁₋₆alkyl-aminosulfonyl; cyano; C₁₋₆alkyl-sulfonylamino; C₁₋₆alkyl-sulfonylamino-C₁₋₆alkyl; N—(C₁₋₆alkyl-sulfonyl)aminocarbonyl; N—(C₁₋₆alkyl-sulfonyl)-N—C₁₋₆alkyl-aminocarbonyl; N—(C₁₋₆alkyl-sulfonyl)-amino-C₁₋₆alkyl; aminocarbonyl-C₁₋₆alkyl; N—C₁₋₆alkyl-aminocarbonyl-C₁₋₆alkyl; N,N-di-C₁₋₆alkyl-aminocarbonyl-C₁₋₆alkyl; C₁₋₆alkoxy-carbonyl; halo-C₁₋₆alkyl; heterocyclyl; or heteroaryl; wherein the C₁₋₆alkyl moieties may be unsubstituted or substituted one or more times with halo; and wherein the heterocyclyl, heteroaryl, C₃₋₆cycloalkyl, C₃₋₆cycloalkenyl and C₃₋₆cycloalkyl-C₁₋₆alkyl moieties may be unsubstituted or substituted one or more times with Rⁱ;

or R^{h} and one of R¹⁰ and R¹¹ together with the atoms to which they are attached may form a four, five, six or seven membered aromatic, partially saturated or unsaturated ring that may optionally include one or two additional heteroatom selected from —O—, —NR^{a}— or —S—, and which may be optionally substituted one or more times with Rⁱ—;

or one of R^{f} and R^{g} and one of R¹⁰ and R¹¹ together with the atoms to which they are attached may form a three, four, five, six or seven membered aromatic, partially saturated or unsaturated ring that may optionally include an additional heteroatom selected from —O—, —NR^{a}— or —S—, and which may be optionally substituted one or more times with Rⁱ;

Rⁱ is: C₁₋₆alkyl; halo; oxo; hydroxy; acetyl; C₁₋₆alkyl-carbonyl; amino-carbonyl; hydroxy-C₁₋₆alkyl; cyano; heteroaryl; or C₁₋₆alkoxy; wherein the C₁₋₆alkyl moieties may be unsubstituted or substituted one or more times with halo; and

R^{j} and R^{k} each independent is: hydrogen; or C₁₋₆alkyl which may be unsubstituted or substituted one or more times with halo.

In certain embodiments of formula I, when A is a heteroatom, then X is —CH—.

In certain embodiments of formula I, when Y is a heteroatom, then q is 1 or 2.

In certain embodiments of formula I, when Y and Z are heteroatom, then q is 2 and r is 2 or 3.

In certain embodiments of formula I, when Z is a heteroatom and A is —(CRⱼRₖ)ₜ—; —NR^{a}—(CRⱼRₖ)ₜ; —O—(CRⱼRₖ)ₜ; —S—(CRⱼRₖ)ₜ; or —SO₂—(CRⱼRₖ)ₜ; then t is from 2 to 4.

In certain embodiments of formula I, m is 0.

In certain embodiments of formula I, m is 1.

In certain embodiments of formula I, n is 0.

In certain embodiments of formula I, n is 1.

In certain embodiments of formula I, p is from 0 to 2.

In certain embodiments of formula I, q is 0.

In certain embodiments of formula I, q is 1.

In certain embodiments of formula I, q is 2.

In certain embodiments of formula I, r is 1.

In certain embodiments of formula I, r is 2.

In certain embodiments of formula I, r is 3.

In certain embodiments of formula I, t is from 0 to 3.

In certain embodiments of formula I, t is 0.

In certain embodiments of formula I, t is 1.

In certain embodiments of formula I, t is 2.

In certain embodiments of formula I, t is 3.

In certain embodiments of formula I, A is: a bond; —CH₂—; —C(O)—; —NR^{a}—; —O—; —S—; or —SO₂—.

In certain embodiments of formula I, A is: a bond; —(CRⱼRₖ)ₜ—; —C(O)—(CRⱼRₖ)ₜ—; —(CRⱼRₖ)ₜ—C(O)—; —(CRⱼRₖ)ₜ—NR^{a}—; —C(O)NR^{a}—(CRⱼRₖ)ₜ—; —(CRⱼRₖ)ₜ—NR^{a}C(O)—; —(CRⱼRₖ)ₜ—O—; —(CRⱼRₖ)ₜ—S—; - or —(CRⱼRₖ)ₜ—SO₂—.

In certain embodiments of formula I, A is: a bond; —C(O)—(CRⱼRₖ)ₜ—; —(CRₜRₖ)ₜ—C(O)—; —(CRₜRₖ)ₜ—NR^{a}—; —C(O)NR^{a}—(CRₜRₖ)ₜ—; (CRⱼRₖ)ₜ—NR^{a}C(O)—; or —(CRⱼRₖ)ₜ—O—.

In certain embodiments of formula I, A is: a bond; —NR^{a}—; —O—; or —S—.

In certain embodiments of formula I, A is: a bond; —NR^{a}—; or —O—.

In certain embodiments of formula I, A is a bond.

In certain embodiments of formula I, A is —CH₂—.

In certain embodiments of formula I, A is —C(O)—.

In certain embodiments of formula I, A is —NR^{a}—.

In certain embodiments of formula I, A is —O—.

In certain embodiments of formula I, A is —S—.

In certain embodiments of formula I, A is —SO₂—.

In certain embodiments of formula I, A is —C(O)NR^{a}—(CH₂)ₜ.

In certain embodiments of formula I, A is —(CH₂)ₜ—NR^{a}C(O)—.

In certain embodiments of formula I, A is —(CRⱼRₖ)ₜ—.

In certain embodiments of formula I, A is —CRⱼRₖ—.

In certain embodiments of formula I, A is —C(O)—(CRⱼRₖ)ₜ—.

In certain embodiments of formula I, A is —(CRⱼRₖ)ₜ—C(O)—.

In certain embodiments of formula I, A is —NR^{a}—(CRₜRₖ)ₜ—.

In certain embodiments of formula I, A is —(CRⱼRₖ)ₜ—NR^{a}—.

In certain embodiments of formula I, A is —C(O)NR^{a}—(CRₜRₖ)ₜ—.

In certain embodiments of formula I, A is (CRⱼRₖ)ₜ—NR^{a}C(O)—.

In certain embodiments of formula I, A is —O—(CRⱼRₖ)ₜ—.

In certain embodiments of formula I, A is —(CRⱼRₖ)ₜ—O—.

In certain embodiments of formula I, A is —S—(CRⱼRₖ)ₜ—.

In certain embodiments of formula I, A is —(CRⱼRₖ)ₜ—S—.

In certain embodiments of formula I, A is —SO₂—(CRⱼRₖ)ₜ—.

In certain embodiments of formula I, A is —(CRⱼRₖ)ₜ—SO₂—.

In certain embodiments of formula I, A is —(CH₂)₂—O—.

In certain embodiments of formula I, A is —(CH₂)—O—.

In certain embodiments of formula I, A is —O—(CH₂)₂—.

In certain embodiments of formula I, A is —O—(CH₂)—.

In certain embodiments of formula I, A is —(CH₂)₂—C(O)—.

In certain embodiments of formula I, A is —(CH₂)—C(O)—.

In certain embodiments of formula I, A is —C(O)—(CH₂)₂—.

In certain embodiments of formula I, A is —C(O)—(CH₂)—.

In certain embodiments of formula I, A is —C(O)—NH—.

In certain embodiments of formula I, A is —CH₂—C(O)—NH—.

In certain embodiments of formula I, A is —NH—.

In certain embodiments of formula I, A is —(CH₂)₂—NH—.

In certain embodiments of formula I, A is —CH₂—NH—.

In certain embodiments of formula I, A is —NH—(CH₂)₂—.

In certain embodiments of formula I, A is —NH—CH₂—.

In certain embodiments of formula I, A is —NH—C(O)—.

In certain embodiments of formula I, t is from 0 to 3.

In certain embodiments of formula I, t is from 1 to 3.

In certain embodiments of formula I, t is from 0 to 2.

In certain embodiments of formula I, t is 0.

In certain embodiments of formula I, t is 1.

In certain embodiments of formula I, t is 2.

In certain embodiments of formula I, t is 3.

In certain embodiments of formula I, t is 4.

In certain embodiments of formula I, W is —CR^{b}R^{c}— or —O—.

In certain embodiments of formula I, W is —CR^{b}R^{c}—.

In certain embodiments of formula I, W is —O—.

In certain embodiments of formula I, W is —NR^{d}—.

In certain embodiments of formula I, W is —S—.

In certain embodiments of formula I, W is —SO₂—.

In certain embodiments of formula I, W is —CH₂—.

In certain embodiments of formula I, one or two of X¹, X², X³ and X⁴ is N and the others are CR^{e}.

In certain embodiments of formula I, three of X¹, X², X³ and X⁴ are CR^{e} and the other is N.

In certain embodiments of formula I, X¹, X², X³ and X⁴ are CR^{e}.

In certain embodiments of formula I, X¹ is N and X², X³ and X⁴ are CR^{e}.

In certain embodiments of formula I, X² is N and X¹, X³ and X⁴ are CR^{e}.

In certain embodiments of formula I, X¹ and X⁴ are N, and X² and X³ are CR^{a}.

In certain embodiments of formula I, X² and X³ are N, and X¹ and X⁴ are CR^{e}.

In certain embodiments of formula I, X¹ and X² are N, and X³ and X⁴ are CR^{e}.

In certain embodiments of formula I, Y is —O—, —CR^{f}R^{g}— or NR^{h}—.

In certain embodiments of formula I, Y is —CR^{f}R^{g}— or —NR^{h}—.

In certain embodiments of formula I, Y is —O—.

In certain embodiments of formula I, Y is —S—.

In certain embodiments of formula I, Y is —SO₂—.

In certain embodiments of formula I, Y is —CR^{f}R^{g}—.

In certain embodiments of formula I, Y is —NR^{h}—.

In certain embodiments of formula I, Z is CH.

In certain embodiments of formula I, Z is N.

In certain embodiments of formula I, each R¹ is independently: C₁₋₆alkyl; halo; C₁₋₆alkoxy; cyano; halo-C₁₋₆alkyl; or halo-C₁₋₆alkoxy.

In certain embodiments of formula I, R¹ is hydrogen.

In certain embodiments of formula I, R¹ is C₁₋₆alkyl.

In certain embodiments of formula I, R² is hydrogen.

In certain embodiments of formula I, R² is C₁₋₆alkyl.

In certain embodiments of formula I, R³ is hydrogen.

In certain embodiments of formula I, R³ is C₁₋₆alkyl.

In certain embodiments of formula I, R⁴ is hydrogen.

In certain embodiments of formula I, R⁴ is C₁₋₆alkyl.

In certain embodiments of formula I, R⁵ is hydrogen.

In certain embodiments of formula I, R⁵ is C₁₋₆alkyl.

In certain embodiments of formula I, R⁶ is hydrogen.

In certain embodiments of formula I, R⁶ is C₁₋₆alkyl.

In certain embodiments of formula I, R⁷ is hydrogen.

In certain embodiments of formula I, R⁷ is C₁₋₆alkyl.

In certain embodiments of formula I, R⁸ is hydrogen.

In certain embodiments of formula I, R⁸ is C₁₋₆alkyl.

In certain embodiments of formula I, R³ and R⁴ together with the atoms to which they are attached form a three, four, five, six or seven membered saturated or partially saturated ring that may optionally include one or two heteroatoms selected from —O—, —NR^{a}— or —S—, and which may be optionally substituted one or more times with Rⁱ.

In certain embodiments of formula I, R³ and R⁴ together with the atoms to which they are attached form a three, four or five membered saturated ring.

In certain embodiments of formula I, R⁵ and R⁶ together with the atoms to which they are attached form a three, four, five, six or seven membered saturated or partially saturated ring that may optionally include one or two heteroatoms selected from —O—, —NR^{a}— or —S—, and which may be optionally substituted one or more times with Rⁱ.

In certain embodiments of formula I, R⁵ and R⁶ together with the atoms to which they are attached form a three, four or five membered saturated ring.

In certain embodiments of formula I, R⁷ and R⁸ together with the atoms to which they are attached form a three, four, five, six or seven membered saturated or partially saturated ring that may optionally include one or two heteroatoms selected from —O—, —NR^{a}— or —S—, and which may be optionally substituted one or more times with Rⁱ.

In certain embodiments of formula I, R⁷ and R⁸ together with the atoms to which they are attached form a three, four or five membered saturated ring.

In certain embodiments of formula I, one of R³ and R⁴ together with one of R⁵ and R⁶ and the atoms to which they are attached form a three, four, five, six or seven membered ring that may optionally include one or two heteroatoms selected from —O—, —NR^{a}— or —S—, and which may be optionally substituted one or more times with Rⁱ.

In certain embodiments of formula I, one of R⁵ and R⁶ together with one of R⁷ and R⁸ and the atoms to which they are attached form a three, four, five, six or seven membered saturated or partially saturated ring that may optionally include one or two heteroatoms selected from —O—, —NR^{a}— or —S—, and which may be optionally substituted one or more times with Rⁱ.

In certain embodiments of formula I, each R⁹ is independently: C₁₋₆alkyl; halo; or halo-C₁₋₆alkyl.

In certain embodiments of formula I, each R⁹ is independently: C₁₋₆alkyl; C₃-cycloalkyl; heterocyclyl; or heteroaryl; each of which may be unsubstituted or substituted one or more times with Rⁱ;

In certain embodiments of formula I, each R⁹ is independently: C₃-cycloalkyl; heterocyclyl; or heteroaryl; each of which may be unsubstituted or substituted one or more times with Rⁱ;

In certain embodiments of formula I, R⁹ is C₁₋₆alkyl which may be unsubstituted or substituted one or more times with W.

In certain embodiments of formula I, R⁹ is C₃-cycloalkyl which may be unsubstituted or substituted one or more times with W.

In certain embodiments of formula I, R⁹ is C₃-cycloalkyl selected from cyclopropyl, cyclobutyl, cyclopentyl and cyclohexyl, each of which may be unsubstituted or substituted one or more times with Rⁱ.

In certain embodiments of formula I, R⁹ is C₃-cycloalkyl selected from cyclopropyl, cyclobutyl, cyclopentyl and cyclohexyl.

In certain embodiments of formula I, R⁹ is cyclohexyl.

In certain embodiments of formula I, R⁹ is heterocyclyl which may be unsubstituted or substituted one or more times with Rⁱ.

In certain embodiments of formula I, R⁹ is heterocyclyl selected from azetidinyl, pyrrolidinyl, piperidinyl, azepinyl, oxetanyl, tetrahydropyranyl and tetrahydropyranyl, each of which may be unsubstituted or substituted one or more times with Rⁱ.

In certain embodiments of formula I, R⁹ is tetrahydropyranyl.

In certain embodiments of formula I, R⁹ is heteroaryl which may be unsubstituted or substituted one or more times with Rⁱ.

In certain embodiments of formula I, R⁹ is heteroaryl selected from thienyl, furanyl, pyrrolyl, pyrazolyl, oxazolyl, thiazolyl, isoxazolyl, isothiazolyl, triazolyl, pyridinyl, or pyrimidinyl, each of which may be unsubstituted or substituted one or more times with Rⁱ.

In certain embodiments of formula I, R⁹ is heteroaryl selected from thienyl, furanyl, pyridinyl, or pyrimidinyl, each of which may be unsubstituted or substituted one or more times with Rⁱ.

In certain embodiments of formula I, R⁹ is heteroaryl selected from thienyl, pyridinyl, or pyrimidinyl, each of which may be unsubstituted or substituted one or more times with Rⁱ.

In certain embodiments of formula I, R⁹ is heteroaryl selected from thienyl, pyridinyl, or pyrimidinyl.

In certain embodiments of formula I, R⁹ is thienyl, pyridinyl, or pyrimidinyl.

In certain embodiments of formula I, R⁹ is pyridinyl.

In certain embodiments of formula I, R⁹ is pyrimidinyl.

In certain embodiments of formula I, R⁹ is thienyl.

In certain embodiments of formula I, R¹⁰ is: hydrogen; halo; or C₁₋₆alkyl which may be unsubstituted or substituted one or more times with halo or oxo.

In certain embodiments of formula I, R¹⁰ is: hydrogen or C₁₋₆alkyl.

In certain embodiments of formula I, R¹⁰ is hydrogen.

In certain embodiments of formula I, R¹⁰ is C₁₋₆alkyl.

In certain embodiments of formula I, R¹⁰ is methyl.

In certain embodiments of formula I, R¹⁰ is halo.

In certain embodiments of formula I, R¹⁰ is carboxy.

In certain embodiments of formula I, R¹⁰ is C₁₋₆alkyl-carbonyl.

In certain embodiments of formula I, R¹⁰ is C₁₋₆alkoxy-carbonyl. In certain embodiments of formula I, R¹⁰ is oxo.

In certain embodiments of formula I, R¹⁰ is hydroxy.

In certain embodiments of formula I, R¹⁰ is aminocarbonyl.

In certain embodiments of formula I, R¹⁰ is N—C₁₋₆alkyl-aminocarbonyl.

In certain embodiments of formula I, R¹⁰ is N,N-di-C₁₋₆alkyl-aminocarbonyl.

In certain embodiments of formula I, R¹⁰ is cyano

In certain embodiments of formula I, R¹⁰ is hydroxy-C₁₆alkyl.

In certain embodiments of formula I, R¹⁰ is N—C₁₋₆alkoxy-C₁₋₆alkyl-aminocarbonyl.

In certain embodiments of formula I, R¹⁰ is N-hydroxy-C₁₋₆alkyl-aminocarbonyl.

In certain embodiments of formula I, R¹⁰ is N—C₁₋₆alkoxy-aminocarbonyl.

In certain embodiments of formula I, R¹¹ is: hydrogen; halo; oxo; hydroxy; or C₁₋₆alkyl which may be unsubstituted or substituted one or more times with halo; or oxo.

In certain embodiments of formula I, R¹¹ is: hydrogen; halo; carboxy; C₁₋₆alkyl-carbonyl; C₁₋₆alkoxy-carbonyl; oxo; hydroxy; aminocarbonyl; N—C₁₋₆alkyl-aminocarbonyl; N,N-di-C₁₋₆alkyl-aminocarbonyl; or C₁₋₆alkyl which may be unsubstituted or substituted one or more times with halo or oxo.

In certain embodiments of formula I, R¹¹ is: hydrogen; halo; or C₁₋₆alkyl.

In certain embodiments of formula I, R¹¹ is: hydrogen; C₁₋₆alkyl; or halo.

In certain embodiments of formula I, R¹¹ is: hydrogen; or C₁₋₆alkyl.

In certain embodiments of formula I, R¹¹ is hydrogen.

In certain embodiments of formula I, R¹¹ is C₁₋₆alkyl

In certain embodiments of formula I, R¹⁰¹ is methyl.

In certain embodiments of formula I, R¹¹ is halo.

In certain embodiments of formula I, R¹¹ is oxo.

In certain embodiments of formula I, R¹¹ is C₁₋₆alkyl-sulfonylamino.

In certain embodiments of formula I, R¹¹ is C₁₋₆alkyl-sulfonylamino-C₁₋₆alkyl.

In certain embodiments of formula I, R¹¹ is cyano.

In certain embodiments of formula I, R¹¹ is hydroxy-C₁₆alkyl.

In certain embodiments of formula I, R¹¹ is N—C₁₋₆alkoxy-C₁₋₆alkyl-aminocarbonyl.

In certain embodiments of formula I, R¹¹ is N-hydroxy-C₁₋₆alkyl-aminocarbonyl.

In certain embodiments of formula I, R¹¹ is N—C₁₋₆alkoxy-aminocarbonyl.

In certain embodiments of formula I, R¹¹ is: hydrogen; or C₁₋₆alkyl.

In certain embodiments of formula I, R¹² is hydrogen.

In certain embodiments of formula I, R¹² is halo.

In certain embodiments of formula I, R¹² is carboxy.

In certain embodiments of formula I, R¹² is C₁₋₆alkyl-carbonyl.

In certain embodiments of formula I, R¹² is C₁₋₆alkoxy-carbonyl.

In certain embodiments of formula I, R¹² is oxo.

In certain embodiments of formula I, R¹² is hydroxy.

In certain embodiments of formula I, R¹² is aminocarbonyl.

In certain embodiments of formula I, R¹² is N—C₁₋₆alkyl-aminocarbonyl.

In certain embodiments of formula I, R¹² is N,N-di-C₁₋₆alkyl-aminocarbonyl.

In certain embodiments of formula I, R¹² is cyano.

In certain embodiments of formula I, R¹² is hydroxy-C₁₆alkyl.

In certain embodiments of formula I, R¹² is N—C₁₋₆alkoxy-C₁₋₆alkyl-aminocarbonyl.

In certain embodiments of formula I, R¹² is N-hydroxy-C₁₋₆alkyl-aminocarbonyl.

In certain embodiments of formula I, R¹² is N—C₁₋₆alkoxy-aminocarbonyl.

In certain embodiments of formula I, R¹² is C₁₋₆alkyl.

In certain embodiments of formula I, R¹² is methyl.

In certain embodiments of formula I, R¹⁰ and R¹¹ together with the atoms to which they are attached form a three, four, five, six or seven membered saturated or partially saturated ring that may optionally include one or two heteroatoms selected from —O—, —NR^{a}— or —S—, and which may be optionally substituted one or more times with Rⁱ.

In certain embodiments of formula I, R¹⁰ and R¹¹ together with the atoms to which they are attached form a four, five, six or seven membered ring;

In certain embodiments of formula I, R^{a} is hydrogen.

In certain embodiments of formula I, R^{a} is C₁₋₆alkyl.

In certain embodiments of formula I, R^{b} is hydrogen.

In certain embodiments of formula I, R^{b} is C₁₋₆alkyl.

In certain embodiments of formula I, R^{c} is hydrogen.

In certain embodiments of formula I, R^{c} is C₁₋₆alkyl.

In certain embodiments of formula I, R^{b} and together with the atoms to which they are attached form a three, four, five, six or seven membered saturated or partially saturated ring that may optionally include one or two heteroatoms selected from —O—, —NR^{a}— or —S—, and which may be optionally substituted one or more times with Rⁱ.

In certain embodiments of formula I, one of R^{b} and together with one of R⁷ and R⁸ and the atoms to which they are attached form a three, four, five, six or seven membered saturated or partially saturated ring that may optionally include one or two heteroatoms selected from —O—, —NR^{a}— or —S—, and which may be optionally substituted one or more times with Rⁱ.

In certain embodiments of formula I, one of R^{b} and R^{c} together with one of R⁵ and R⁶ and the atoms to which they are attached form a three, four, five, six or seven membered saturated or partially saturated ring that may optionally include one or two heteroatoms selected from —O—, —NR^{a}— or —S—, and which may be optionally substituted one or more times with Rⁱ.

In certain embodiments of formula I, R^{d} is hydrogen.

In certain embodiments of formula I, R^{d} is C₁₋₆alkyl.

In certain embodiments of formula I, each R^{e} is independently: hydrogen; C₁₋₆alkyl; halo; or halo-C₁₋₆alkyl.

In certain embodiments of formula I, each R^{e} is independently: hydrogen; C₁₋₆alkyl; or halo.

In certain embodiments of formula I, each R^{e} is independently: hydrogen; or halo.

In certain embodiments of formula I, each R^{e} is independently: hydrogen; or fluoro.

In certain embodiments of formula I, R^{e} is hydrogen.

In certain embodiments of formula I, R^{e} is C₁₋₆alkyl.

In certain embodiments of formula I, R^{e} is halo.

In certain embodiments of formula I, R^{e} is C₁₋₆alkoxy.

In certain embodiments of formula I, R^{e} is cyano.

In certain embodiments of formula I, R^{e} is halo-C₁₋₆alkyl.

In certain embodiments of formula I, each R^{f} is independently: hydrogen; or C₁₋₆alkyl.

In certain embodiments of formula I, R^{f} is hydrogen.

In certain embodiments of formula I, R^{f} is C₁₋₆alkyl.

In certain embodiments of formula I, R^{f} is halo.

In certain embodiments of formula I, R^{g} is: C₁₋₆alkyl; C₃₋₆cycloalkyl; C₃₋₆cycloalkenyl; C₃₋₆cycloalkyl-C₁₋₆alkyl; halo; C₁₋₆alkyl-carbonyl; C₃₋₆cycloalkyl-carbonyl; C₃₋₆cycloalkyl-C₁₋₆alkyl-carbonyl; cyano-C₁₋₆alkyl-carbonyl; hydroxy-C₁₋₆alkyl-carbonyl; C₁₋₆alkoxy-C₁₋₆alkyl-carbonyl; carboxy; N-cyano-aminocarbonyl; N-cyano-N—C₁₋₆alkyl-aminocarbonyl; N—C₁₋₆alkyl-acetimidamidyl; N,N′-di-C₁₋₆alkyl-acetimidamidyl; N′-cyano-N—C₁₋₆alkyl-acetimidamidyl; N′-hydroxy-acetimidamidyl; N′—C₁₋₆alkoxy-acetimidamidyl; N′-hydroxy-N—C₁₋₆alkyl-acetimidamidyl; N′—C₁₋₆alkoxy-N—C₁₋₆alkyl-acetimidamidyl; 2-nitro-1-N—C₁₋₆alkylamino-vinyl; formyl; C₁₋₆alkyl-sulfonyl; C₃₋₆cycloalkyl-sulfonyl; C₃₋₆cycloalkyl-C₁₋₆alkyl-sulfonyl; C₁₋₆alkyl-sulfonyl-C₁₋₆alkyl; aminocarbonyl; N-hydroxy-aminocarbonyl; N—C₁₋₆alkoxy-aminocarbonyl; N—C₁₋₆alkyl-aminocarbonyl; N-hydroxy-N—C₁₋₆alkyl-aminocarbonyl; N—C₁₋₆alkoxy-N—C₁₋₆alkyl-aminocarbonyl; N,N-di-C₁₋₆alkyl-aminocarbonyl; aminosulfonyl; N—C₁₋₆alkyl-aminosulfonyl; N,N-di-C₁₋₆alkyl-aminosulfonyl; cyano; C₁₋₆alkoxy; C₁₋₆alkyl-sulfonylamino; N—C₁₋₆alkyl-sulfonylaminocarbonyl; N—(C₁₋₆alkyl-sulfonyl)-N—C₁₋₆alkyl-aminocarbonyl; N—(C₁₋₆alkyl-sulfonyl)-amino-C₁₋₆alkyl; amino; N—C₁₋₆alkyl-amino; N,N-di-C₁₋₆alkyl-amino; halo-C₁₋₆alkyl; heterocyclyl; heteroaryl; or hydroxyl; wherein the C₁₋₆alkyl moieties may be unsubstituted or substituted one or more times with halo; and wherein the heterocyclyl, heteroaryl, C₃₋₆cycloalkyl, C₃₋₆cycloalkenyl and C₃₋₆cycloalkyl-C₁₋₆alkyl moieties may be unsubstituted or substituted one or more times with Rⁱ.

In certain embodiments of formula I, R^{g} is: hydrogen; C₁₋₆alkyl; C₃₋₆cycloalkyl; C₃₋₆cycloalkyl-C₁₋₆alkyl; halo; C₁₋₆alkyl-carbonyl; C₃₋₆cycloalkyl-carbonyl; C₃₋₆cycloalkyl-C₁₋₆alkyl-carbonyl; C₁₋₆alkyl-sulfonyl; C₃₋₆cycloalkyl-sulfonyl; C₃₋₆cycloalkyl-C₁₋₆alkyl-sulfonyl; aminocarbonyl; N—C₁₋₆alkyl-aminocarbonyl; N,N-di-C₁₋₆alkyl-aminocarbonyl; aminosulfonyl; N—C₁₋₆alkyl-aminosulfonyl; N,N-di-C₁₋₆alkyl-aminosulfonyl; cyano; C₁₋₆alkoxy; C₁₋₆alkyl-sulfonylamino; amino; N—C₁₋₆alkyl-amino; N,N-di-C₁₋₆alkyl-amino; halo-C₁₋₆alkyl; or hydroxyl; wherein the C₁₋₆alkyl moieties may be unsubstituted or substituted one or more times with halo; and wherein the C₃₋₆cycloalkyl, and C₃₋₆cycloalkyl-C₁₋₆alkyl moieties may be unsubstituted or substituted one or more times with Rⁱ.

In certain embodiments of formula I, R^{g} is hydrogen.

In certain embodiments of formula I, R^{g} is C₁₋₆alkyl.

In certain embodiments of formula I, R^{g} is C₃₋₆cycloalkyl which may be unsubstituted or substituted one or more times with Rⁱ.

In certain embodiments of formula I, R^{g} is C₃₋₆cycloalkyl-C₁₋₆alkyl which may be unsubstituted or substituted one or more times with Rⁱ.

In certain embodiments of formula I, R^{g} is halo.

In certain embodiments of formula I, R^{g} is C₁₋₆alkyl-carbonyl.

In certain embodiments of formula I, R^{g} is C₃₋₆cycloalkyl-carbonyl wherein the C₃₋₆cycloalkyl moeity may be unsubstituted or substituted one or more times with Rⁱ.

In certain embodiments of formula I, R^{g} is C₃₋₆cycloalkyl-C₁₋₆alkyl-carbonyl wherein the C₃₋₆cycloalkyl-C₁₋₆alkyl moiety may be unsubstituted or substituted one or more times with W.

In certain embodiments of formula I, R^{g} is C₁₋₆alkyl-sulfonyl.

In certain embodiments of formula I, R^{g} is C₃₋₆cycloalkyl-sulfonyl.

In certain embodiments of formula I, R^{g} is C₃₋₆cycloalkyl-C₁₋₆alkyl-sulfonyl.

In certain embodiments of formula I, R^{g} is aminocarbonyl.

In certain embodiments of formula I, R^{g} is N—C₁₋₆alkyl-aminocarbonyl.

In certain embodiments of formula I, R^{g} is N,N-di-C₁₋₆alkyl-aminocarbonyl.

In certain embodiments of formula I, R^{g} is aminosulfonyl.

In certain embodiments of formula I, R^{g} is N—C₁₋₆alkyl-aminosulfonyl.

In certain embodiments of formula I, R^{g} is N,N-di-C₁₋₆alkyl-aminosulfonyl.

In certain embodiments of formula I, R^{g} is cyano.

In certain embodiments of formula I, R^{g} is C₁₋₆alkoxy.

In certain embodiments of formula I, R^{g} is C₁₋₆alkyl-sulfonylamino.

In certain embodiments of formula I, R^{g} is amino.

In certain embodiments of formula I, R^{g} is N—C₁₋₆alkyl-amino.

In certain embodiments of formula I, R^{g} is N,N-di-C₁₋₆alkyl-amino.

In certain embodiments of formula I, R^{g} is halo-C₁₋₆alkyl.

In certain embodiments of formula I, R^{g} is hydroxy.

In certain embodiments of formula I, R^{g} is C₃₋₆cycloalkenyl which may be unsubstituted or substituted one or more times with Rⁱ.

In certain embodiments of formula I, R^{g} is cyano-C₁₋₆alkyl-carbonyl.

In certain embodiments of formula I, R^{g} is hydroxy-C₁₋₆alkyl-carbonyl.

In certain embodiments of formula I, R^{g} is C₁₋₆alkoxy-C₁₋₆alkyl-carbonyl.

In certain embodiments of formula I, R^{g} is carboxy.

In certain embodiments of formula I, R^{g} is N-cyano-aminocarbonyl.

In certain embodiments of formula I, R^{g} is N-cyano-N—C₁₋₆alkyl-aminocarbonyl.

In certain embodiments of formula I, R^{g} is N—C₁₋₆alkyl-acetimidamidyl.

In certain embodiments of formula I, R^{g} is N,N′-di-C₁₋₆alkyl-acetimidamidyl.

In certain embodiments of formula I, R^{g} is N′-cyano-N—C₁₋₆alkyl-acetimidamidyl.

In certain embodiments of formula I, R^{g} is N′-hydroxy-acetimidamidyl.

In certain embodiments of formula I, R^{g} is N′—C₁₋₆alkoxy-acetimidamidyl.

In certain embodiments of formula I, R^{g} is N′-hydroxy-N—C₁₋₆alkyl-acetimidamide; N′—C₁₋₆alkoxy-N—C₁₋₆alkyl-acetimidamidyl.

In certain embodiments of formula I, R^{g} is 2-nitro-1-N—C₁₋₆alkylamino-vinyl.

In certain embodiments of formula I, R^{g} is C₁₋₆alkyl-sulfonyl-C₁₋₆alkyl.

In certain embodiments of formula I, R^{g} is N-hydroxy-aminocarbonyl.

In certain embodiments of formula I, R^{g} is N—C₁₋₆alkoxy-aminocarbonyl.

In certain embodiments of formula I, R^{g} is N-hydroxy-N—C₁₋₆alkyl-aminocarbonyl.

In certain embodiments of formula I, R^{g} is N—C₁₋₆alkoxy-N—C₁₋₆alkyl-aminocarbonyl.

In certain embodiments of formula I, R^{g} is N—C₁₋₆alkyl-sulfonylaminocarbonyl.

In certain embodiments of formula I, R^{g} is N—(C₁₋₆alkyl-sulfonyl)-N—C₁₋₆alkyl-aminocarbonyl.

In certain embodiments of formula I, R^{g} is aminocarbonyl-C₁₋₆alkyl.

In certain embodiments of formula I, R^{g} is N—C₁₋₆alkyl-aminocarbonyl-C₁₋₆alkyl

In certain embodiments of formula I, R^{g} is N,N-di-C₁₋₆alkyl-aminocarbonyl-C₁₋₆alkyl.

In certain embodiments of formula I, R^{g} is C₁₋₆alkoxy-carbonyl.

In certain embodiments of formula I, R^{g} is heterocyclyl which may be unsubstituted or substituted one or more times with Rⁱ.

In embodiments of formula I wherein R^{g} is heterocyclyl, such heterocyclyl may be oxetanyl, tetrahydrofuranyl, tetrahydropyranyl, azetidinyl, pyrrolidinyl, piperidinyl, azepinyl or piperazinyl, each of which may be unsubstituted or substituted one or more times with Rⁱ.

In certain embodiments of formula I, R^{g} is heteroaryl which may be unsubstituted or substituted one or more times with Rⁱ.

In embodiments of formula I wherein R^{g} is heteroaryl, such heteroaryl may be pyridinyl, pyrimidinyl, triazinyl, pyrrolyl, imidazolyl, pyrazoyl, triazolyl, oxazolyl, thiazolyl, isoxazolyl, isothiazolyl, oxadiazolyl, thiadiazolyl or tetrazolyl, each of which may be unsubstituted or substituted one or more times with Rⁱ.

In embodiments of formula I wherein R^{g} is heteroaryl, such heteroaryl may be imidazolyl, pyrazoyl, triazolyl, oxazolyl, thiazolyl, isoxazolyl, isothiazolyl, oxadiazolyl, thiadiazolyl or tetrazolyl, each of which may be unsubstituted or substituted one or more times with Rⁱ.

In certain embodiments of formula I, R^{g} is triazolyl.

In certain embodiments of formula I, R^{g} is [1,2,4]triazol-4-yl.

In certain embodiments of formula I, R^{g} is [1,2,4]triazol-3-yl.

In certain embodiments of formula I, R^{g} is 4-methyl-[1,2,4]triazol-3-yl.

In certain embodiments of formula I, R^{g} is [1,2,4]triazol-1-yl.

In certain embodiments of formula I, R^{g} is [1,2,3]triazol-1-yl.

In certain embodiments of formula I, R^{g} is [1,2,3]triazol-4-yl.

In certain embodiments of formula I, R^{g} is 4-methyl-[1,2,4]triazol-3-yl.

In certain embodiments of formula I, R^{g} is pyrazolyl.

In certain embodiments of formula I, R^{g} is pyrazol-3-yl.

In certain embodiments of formula I, R^{g} is pyrazol-1-yl.

In certain embodiments of formula I, R^{g} is pyrazol-4-yl.

In certain embodiments of formula I, R^{g} is imidazolyl.

In certain embodiments of formula I, R^{g} is imidazol-1-yl.

In certain embodiments of formula I, R^{g} is 1-methyl-imidazol-2-yl.

In certain embodiments of formula I, R^{g} is isoxazolyl.

In certain embodiments of formula I, R^{g} is 3-hydroxyisoxazol-5-yl.

In certain embodiments of formula I, R^{g} is oxdiazolyl.

In certain embodiments of formula I, R^{g} is [1,2,4]oxadiazol-5-yl.

In certain embodiments of formula I, R^{g} is [1,2,4]oxadiazol-3-yl.

In certain embodiments of formula I, R^{g} is [1,2,3]oxadiazol-2-yl.

In certain embodiments of formula I, R^{g} is [1,2,3]oxadiazol-2-one-5-yl.

In certain embodiments of formula I, R^{g} is tetrazolyl.

In certain embodiments of formula I, R^{g} is tetrazol-5-yl.

In certain embodiments of formula I, R^{g} is tetrazol-1-yl.

In certain embodiments of formula I, R^{g} is tetrazol-2-yl.

In certain embodiments of formula I, R^{g} is pyrazolyl.

In certain embodiments of formula I, R^{g} is pyridazinyl.

In certain embodiments of formula I, R^{g} is triazinyl.

In certain embodiments of formula I, R^{f} and R^{g} together with the atoms to which they are attached form a three, four, five, six or seven membered saturated or partially saturated ring.

In certain embodiments of formula I, R^{f} and R^{g} together with the atoms to which they are attached form a three membered ring.

In certain embodiments of formula I, R^{f} and R^{g} together with the atoms to which they are attached form a four membered ring.

In certain embodiments of formula I, R^{f} and R^{g} together with the atoms to which they are attached form a five membered ring.

In certain embodiments of formula I, R^{f} and R^{g} together with the atoms to which they are attached form a six membered ring.

In certain embodiments of formula I, R^{f} and R^{g} together with the atoms to which they are attached form a seven membered ring.

In certain embodiments of formula I, R^{h} is: hydrogen; C₁₋₆alkyl; C₃₋₆cycloalkyl; C₃₋₆cycloalkenyl; C₃₋₆cycloalkyl-C₁₋₆alkyl; C₁₋₆alkyl-carbonyl; C₃₋₆cycloalkyl-carbonyl; C₃₋₆cycloalkyl-C₁₋₆alkyl-carbonyl; cyano-C₁₋₆alkyl-carbonyl; hydroxy-C₁₋₆alkyl-carbonyl; C₁₋₆alkoxy-C₁₋₆alkyl-carbonyl; N-cyano-aminocarbonyl; N-cyano-N—C₁₋₆alkyl-aminocarbonyl; N—C₁₋₆alkyl-acetimidamidyl; N,N′-di-C₁₋₆alkyl-acetimidamidyl; N′-cyano-N—C₁₋₆alkyl-acetimidamidyl; N′-hydroxy-acetimidamidyl; N′—C₁₋₆alkoxy-acetimidamidyl; N′-hydroxy-N—C₁₋₆alkyl-acetimidamidyl; N′—C₁₋₆alkoxy-N—C₁₋₆alkyl-acetimidamidyl; formyl; C₁₋₆alkyl-sulfonyl; C₃₋₆cycloalkyl-sulfonyl; C₃₋₆cycloalkyl-C₁₋₆alkyl-sulfonyl; C₁₋₆alkyl-sulfonyl-C₁₋₆alkyl; aminocarbonyl; N-hydroxy-aminocarbonyl; N—C₁₋₆alkoxy-aminocarbonyl; N—C₁₋₆alkyl-aminocarbonyl; N-hydroxy-N—C₁₋₆alkyl-aminocarbonyl; N—C₁₋₆alkoxy-N—C₁₋₆alkyl-aminocarbonyl; N,N-di-C₁₋₆alkyl-aminocarbonyl; aminosulfonyl; N—C₁₋₆alkyl-aminosulfonyl; N,N-di-C₁₋₆alkyl-aminosulfonyl; cyano; C₁₋₆alkyl-sulfonylamino; C₁₋₆alkyl-sulfonylamino-C₁₋₆alkyl; N—(C₁₋₆alkyl-sulfonyl)aminocarbonyl; N—(C₁₋₆alkyl-sulfonyl)-N—C₁₋₆alkyl-aminocarbonyl; N—(C₁₋₆alkyl-sulfonyl)-amino-C₁₋₆alkyl; halo-C₁₋₆alkyl; heterocyclyl; or heteroaryl; wherein the C₁₋₆alkyl moieties may be unsubstituted or substituted one or more times with halo; and wherein the heterocyclyl, heteroaryl, C₃₋₆cycloalkyl, C₃₋₆cycloalkenyl and C₃₋₆cycloalkyl-C₁₋₆alkyl moieties may be unsubstituted or substituted one or more times with Rⁱ.

In certain embodiments of formula I, R^{h} is: hydrogen; C₁₋₆alkyl; C₃₋₆cycloalkyl; C₃₋₆cycloalkyl-C₁₋₆alkyl; C₁₋₆alkyl-carbonyl; C₃₋₆cycloalkyl-carbonyl; C₃₋₆cycloalkyl-C₁₋₆alkyl-carbonyl; C₁₋₆alkyl-sulfonyl; C₃₋₆cycloalkyl-sulfonyl; C₃₋₆cycloalkyl-C₁₋₆alkyl-sulfonyl; aminocarbonyl; N—C₁₋₆alkyl-aminocarbonyl; N,N-di-C₁₋₆alkyl-aminocarbonyl; aminosulfonyl; N—C₁₋₆alkyl-aminosulfonyl; or N,N-di-C₁₋₆alkyl-aminosulfonyl; wherein the C₁₋₆alkyl moieties may be unsubstituted or substituted one or more times with halo; and wherein the C₃₋₆cycloalkyl, and C₃₋₆cycloalkyl-C₁₋₆alkyl moieties may be unsubstituted or substituted one or more times with Rⁱ.

In certain embodiments of formula I, R^{h} is: C₁₋₆alkyl-carbonyl; C₃₋₆cycloalkyl-carbonyl; C₃₋₆cycloalkyl-C₁₋₆alkyl-carbonyl; C₁₋₆alkyl-sulfonyl; C₃₋₆cycloalkyl-sulfonyl; C₃₋₆cycloalkyl-C₁₋₆alkyl-sulfonyl; aminocarbonyl; N—C₁₋₆alkyl-aminocarbonyl; or N,N-di-C₁₋₆alkyl-aminocarbonyl; aminosulfonyl; N—C₁₋₆alkyl-aminosulfonyl; or N,N-di-C₁₋₆alkyl-aminosulfonyl; wherein the C₃₋₆cycloalkyl, and C₃₋₆cycloalkyl-C₁₋₆alkyl moieties each may be unsubstituted or substituted one or more times with Rⁱ.

In certain embodiments of formula I, R^{h} is: C₁₋₆alkyl-carbonyl; C₃₋₆cycloalkyl-carbonyl; C₃₋₆cycloalkyl-C₁₋₆alkyl-carbonyl; C₁₋₆alkyl-sulfonyl; C₃₋₆cycloalkyl-sulfonyl; or C₃₋₆cycloalkyl-C₁₋₆alkyl-sulfonyl; wherein the C₃₋₆cycloalkyl, and C₃₋₆cycloalkyl-C₁₋₆alkyl moieties each may be unsubstituted or substituted one or more times with Rⁱ.

In certain embodiments of formula I, R^{h} is: C₁₋₆alkyl-carbonyl; C₃₋₆cycloalkyl-carbonyl; or C₃₋₆cycloalkyl-C₁₋₆alkyl-carbonyl; wherein the C₃₋₆cycloalkyl, and C₃₋₆cycloalkyl-C₁₋₆alkyl moieties each may be unsubstituted or substituted one or more times with Rⁱ.

In certain embodiments of formula I, R^{h} is hydrogen.

In certain embodiments of formula I, R^{h} is C₁₋₆alkyl.

In certain embodiments of formula I, R^{h} is C₃₋₆cycloalkyl which may be unsubstituted or substituted one or more times with Rⁱ.

In certain embodiments of formula I, R^{h} is C₃₋₆cycloalkyl-C₁₋₆alkyl.

In certain embodiments of formula I, R^{h} is C₁₋₆alkyl-carbonyl.

In certain embodiments of formula I, R^{h} is C₃₋₆cycloalkyl-carbonyl.

In certain embodiments of formula I, R^{h} is C₃₋₆cycloalkyl-C₁₋₆alkyl-carbonyl.

In certain embodiments of formula I, R^{h} is C₁₋₆alkyl-sulfonyl.

In certain embodiments of formula I, R^{h} is C₃₋₆cycloalkyl-sulfonyl.

In certain embodiments of formula I, R^{h} is C₃₋₆cycloalkyl-C₁₋₆alkyl-sulfonyl.

In certain embodiments of formula I, R^{h} is aminocarbonyl.

In certain embodiments of formula I, R^{h} is N—C₁₋₆alkyl-aminocarbonyl.

In certain embodiments of formula I, R^{h} is N,N-di-C₁₋₆alkyl-aminocarbonyl.

In certain embodiments of formula I, R^{h} is aminosulfonyl.

In certain embodiments of formula I, R^{h} is N—C₁₋₆alkyl-aminosulfonyl.

In certain embodiments of formula I, R^{h} is or N,N-di-C₁₋₆alkyl-aminosulfonyl.

In certain embodiments of formula I, R^{h} is C₃₋₆cycloalkenyl.

In certain embodiments of formula I, R^{h} is cyano-C₁₋₆alkyl-carbonyl.

In certain embodiments of formula I, R^{h} is hydroxy-C₁₋₆alkyl-carbonyl.

In certain embodiments of formula I, R^{h} is C₁₋₆alkoxy-C₁₋₆alkyl-carbonyl.

In certain embodiments of formula I, R^{h} is N-cyano-aminocarbonyl.

In certain embodiments of formula I, R^{h} is N-cyano-N—C₁₋₆alkyl-aminocarbonyl.

In certain embodiments of formula I, R^{h} is N—C₁₋₆alkyl-acetimidamidyl.

In certain embodiments of formula I, R^{h} is N,N′-di-C₁₋₆alkyl-acetimidamidyl.

In certain embodiments of formula I, R^{h} is N′-cyano-N—C₁₋₆alkyl-acetimidamidyl.

In certain embodiments of formula I, R^{h} is N′-hydroxy-acetimidamidyl.

In certain embodiments of formula I, R^{h} is N′—C₁₋₆alkoxy-acetimidamidyl.

In certain embodiments of formula I, R^{h} is N′-hydroxy-N—C₁₋₆alkyl-acetimidamidyl.

In certain embodiments of formula I, R^{h} is N′—C₁₋₆alkoxy-N—C₁₋₆alkyl-acetimidamidyl.

In certain embodiments of formula I, R^{h} is 2-nitro-1-N—C₁₋₆alkylamino-vinyl.

In certain embodiments of formula I, R^{h} is C₁₋₆alkyl-sulfonyl-C₁₋₆alkyl.

In certain embodiments of formula I, R^{h} is N-hydroxy-aminocarbonyl.

In certain embodiments of formula I, R^{h} is N—C₁₋₆alkoxy-aminocarbonyl.

In certain embodiments of formula I, R^{h} is N-hydroxy-N—C₁₋₆alkyl-aminocarbonyl.

In certain embodiments of formula I, R^{h} is N—C₁₋₆alkoxy-N—C₁₋₆alkyl-aminocarbonyl.

In certain embodiments of formula I, R^{h} is C₁₋₆alkyl-sulfonylamino-C₁₋₆alkyl.

In certain embodiments of formula I, R^{h} is N—(C₁₋₆alkyl-sulfonyl)aminocarbonyl.

In certain embodiments of formula I, R^{h} is N—(C₁₋₆alkyl-sulfonyl)-N—C₁₋₆alkyl-aminocarbonyl.

In certain embodiments of formula I, R^{h} is aminocarbonyl-C₁₋₆alkyl.

In certain embodiments of formula I, R^{h} is N—C₁₋₆alkyl-aminocarbonyl-C₁₋₆alkyl

In certain embodiments of formula I, R^{h} is N,N-di-C₁₋₆alkyl-aminocarbonyl-C₁₋₆alkyl.

In certain embodiments of formula I, R^{h} is C₁₋₆alkoxy-carbonyl.

In certain embodiments of formula I, R^{h} is heterocyclyl which may be unsubstituted or substituted one or more times with Rⁱ.

In certain embodiments of formula I, R^{h} is heteroaryl which may be unsubstituted or substituted one or more times with Rⁱ.

In embodiments of formula I wherein R^{h} is heteroaryl, such heteroaryl may be pyridinyl, pyrimidinyl, pyrolyl, imidazolyl, pyrazoyl, triazolyl, oxazolyl, thiazolyl, isoxazolyl, isothiazolyl, oxadiazolyl, thiadiazolyl or tetrazolyl, each of which may be unsubstituted or substituted one or more times with Rⁱ.

In embodiments of formula I wherein R^{h} is heteroaryl, such heteroaryl may be imidazolyl, pyrazoyl, triazolyl, oxazolyl, thiazolyl, isoxazolyl, isothiazolyl, oxadiazolyl, thiadiazolyl or tetrazolyl, each of which may be unsubstituted or substituted one or more times with Rⁱ.

In certain embodiments of formula I, R^{h} is acetyl.

In certain embodiments of formula I, R^{h} is methanesulfonyl.

In certain embodiments of formula I, R^{h} is cyclopropylcarbonyl.

In certain embodiments of formula I, R^{h} and one of R¹⁰ and R¹¹ together with the atoms to which they are attached form a four, five, six or seven membered aromatic, partially saturated or unsaturated ring.

In certain embodiments of formula I, R^{h} and one of R¹⁰ and R¹¹ together with the atoms to which they are attached form a four membered ring.

In certain embodiments of formula I, R^{h} and one of R¹⁰ and R¹¹ together with the atoms to which they are attached form a five membered ring.

In certain embodiments of formula I, R^{h} and one of R¹⁰ and R¹¹ together with the atoms to which they are attached form a six membered ring.

In certain embodiments of formula I, R^{h} and one of R¹⁰ and R¹¹ together with the atoms to which they are attached form a seven membered ring.

In certain embodiments of formula I, one of R^{f} and R^{g} and one of R¹⁰ and R¹¹ together with the atoms to which they are attached form a four, five, six or seven membered aromatic, partially saturated or unsaturated ring.

In certain embodiments of formula I, one of R^{f} and R^{g} and one of R¹⁰ and R¹¹ together with the atoms to which they are attached form a five or six membered aromatic ring.

In certain embodiments of formula I, one of R^{f} and R^{g} and one of R¹⁰ and R¹¹ together with the atoms to which they are attached form a five membered aromatic ring.

In certain embodiments of formula I, one of R^{f} and R^{g} and one of R¹⁰ and R¹¹ together with the atoms to which they are attached form a six membered aromatic ring.

In certain embodiments of formula I, one of R^{f} and R^{g} and one of R¹⁰ and R¹¹ together with the atoms to which they are attached form a five or six membered saturated ring.

In certain embodiments of formula I, one of R^{f} and R^{g} and one of R¹⁰ and R¹¹ together with the atoms to which they are attached form a five membered saturated ring.

In certain embodiments of formula I, one of R^{f} and R^{g} and one of R¹⁰ and R¹¹ together with the atoms to which they are attached form a six membered saturated ring.

In certain embodiments of formula I, one of R^{f} and R^{g} and one of R¹⁰ and R¹¹ together with the atoms to which they are attached form a four membered ring.

In certain embodiments of formula I, one of R^{f} and R^{g} and one of R¹⁰ and R¹¹ together with the atoms to which they are attached form a five membered ring.

In certain embodiments of formula I, one of R^{f} and R^{g} and one of R¹⁰ and R¹¹ together with the atoms to which they are attached form a six membered ring.

In certain embodiments of formula I, one of R^{f} and R^{g} and one of R¹⁰ and R¹¹ together with the atoms to which they are attached form a seven membered ring.

In certain embodiments of formula I, Rⁱ is: C₁₋₆alkyl; halo; oxo; hydroxy; acetyl; or C₁₋₆alkoxy.

In certain embodiments of formula I, Rⁱ is C₁₋₆alkyl.

In certain embodiments of formula I, Rⁱ is halo.

In certain embodiments of formula I, Rⁱ is C₁₋₆alkoxy.

In certain embodiments of formula I, Rⁱ is halo-C₁₋₆alkyl.

In certain embodiments of formula I, Rⁱ is oxo.

In certain embodiments of formula I, Rⁱ is hydroxy.

In certain embodiments of formula I, Rⁱ is acetyl.

In certain embodiments of formula I, Rⁱ is C₁₋₆alkyl-carbonyl.

In certain embodiments of formula I, Rⁱ is amino-carbonyl.

In certain embodiments of formula I, Rⁱ is hydroxy-C₁₋₆alkyl.

In certain embodiments of formula I, Rⁱ is cyano.

In certain embodiments of formula I, Rⁱ is heteroaryl;

In certain embodiments of formula I, R^{j} and R^{k} each independent is: hydrogen; or methyl.

In certain embodiments of formula I, Rⱼ is hydrogen.

In certain embodiments of formula I, R^{k} is hydrogen.

In certain embodiments of the invention, the group

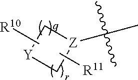

is:

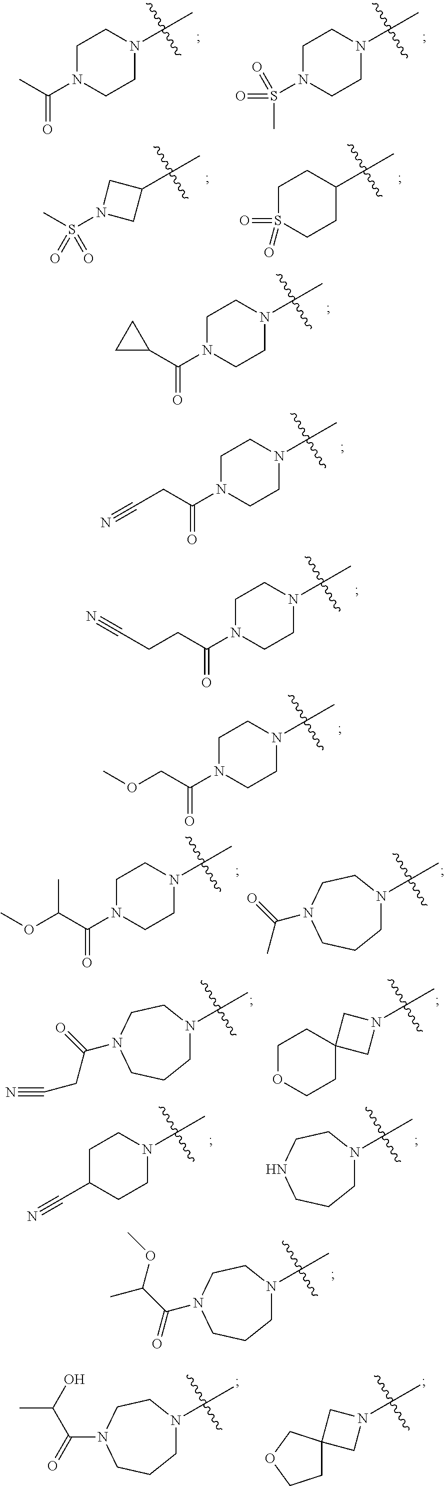

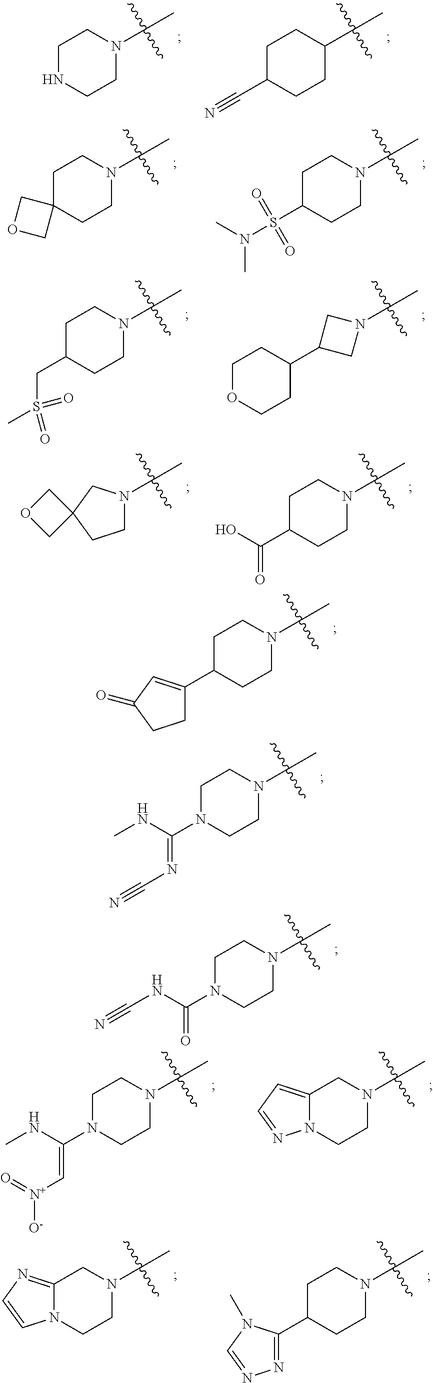

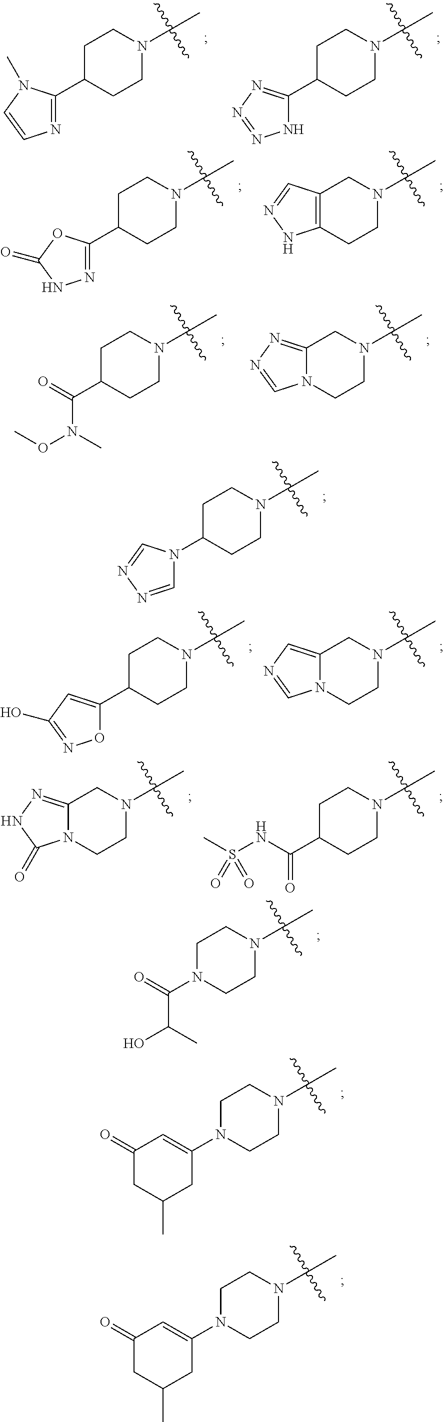

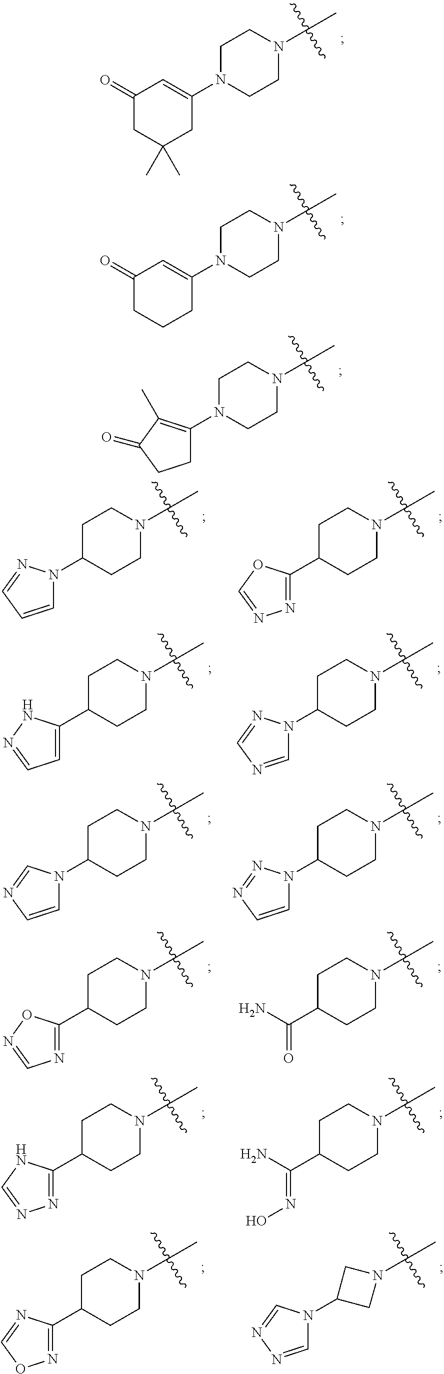

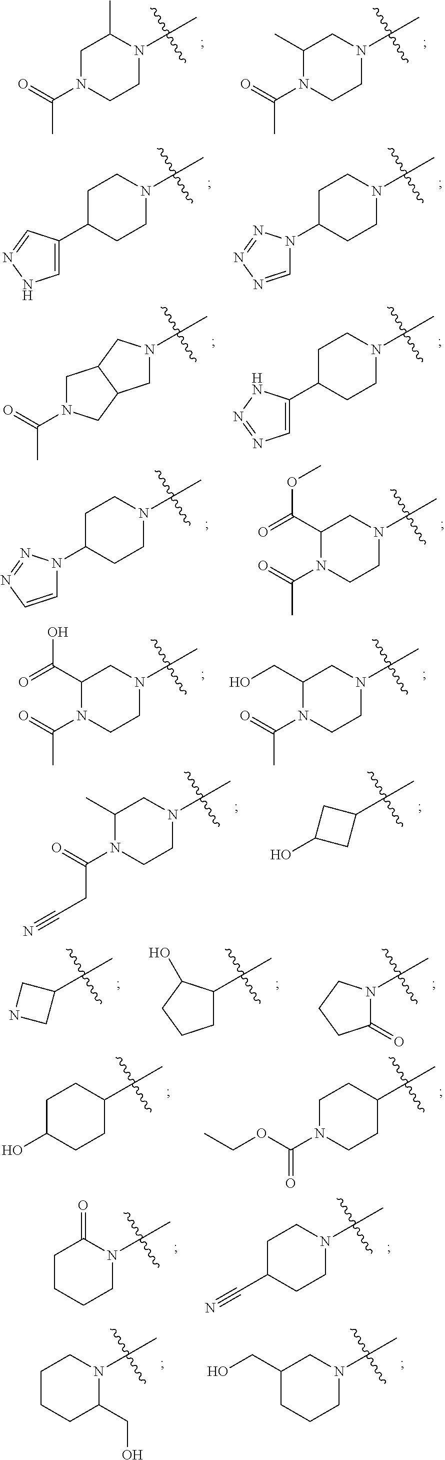

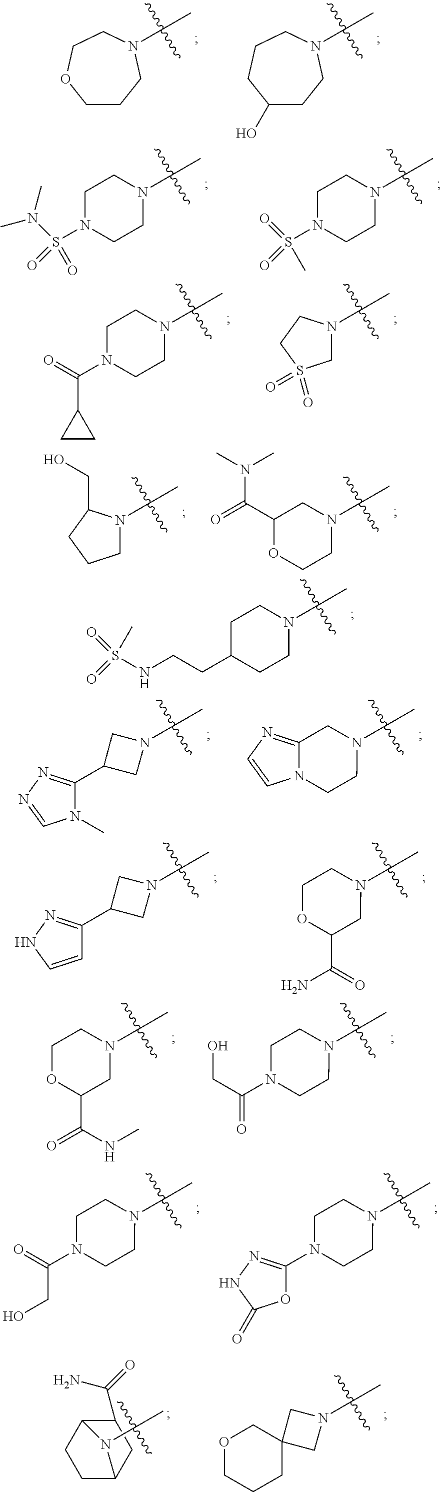

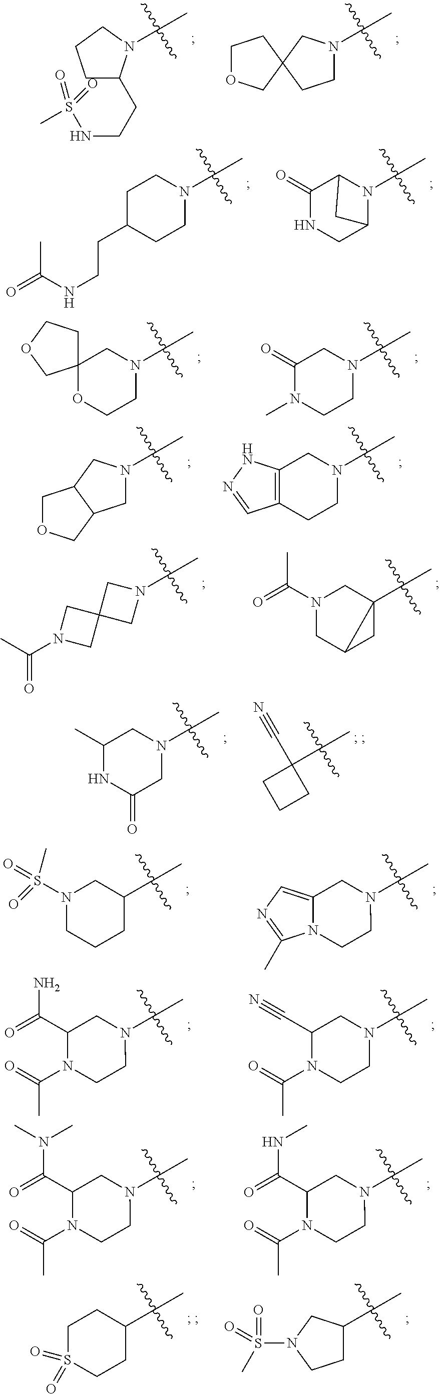

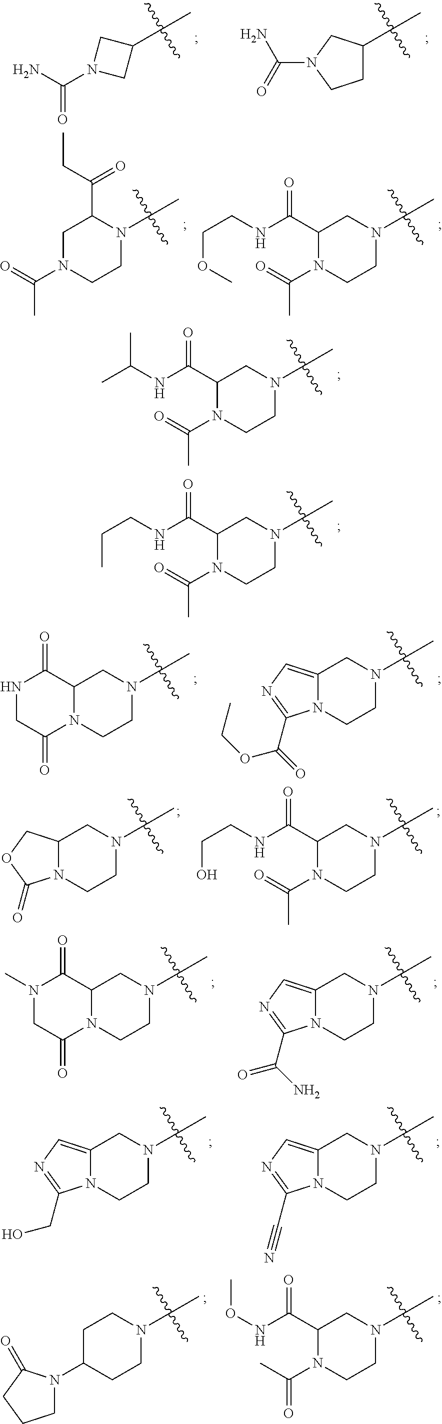

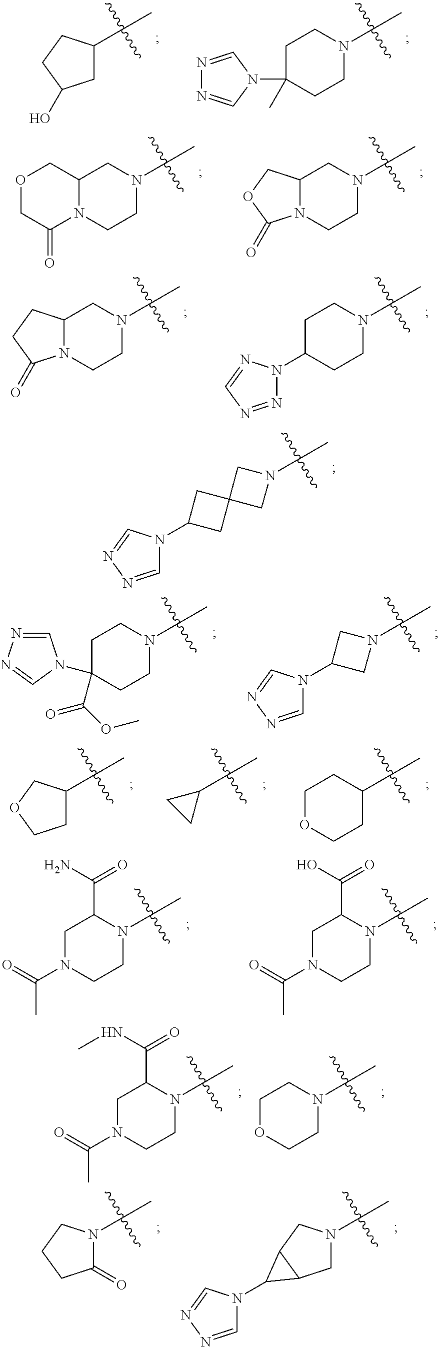

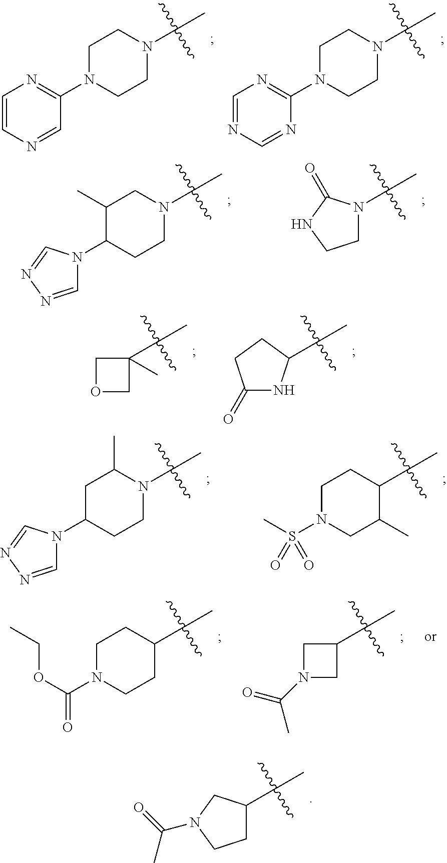

In certain embodiments of the invention, the group

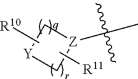

is:

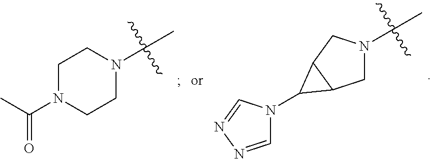

In certain embodiments of formula I, the subject compounds may be of formula Ia or Ib:

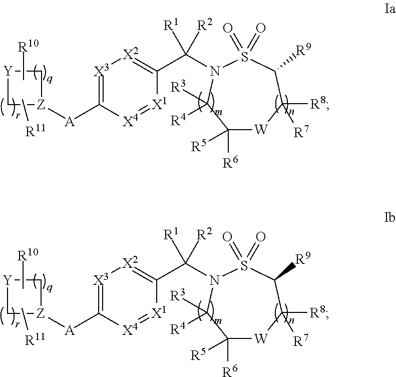

wherein m, n, r, A, W, X¹, X², X³, X⁴, Y, Z, R¹, R², R³, R⁴, R⁵, R⁶, R⁷, R⁸, R⁹, R¹⁰ and R¹¹, and the group

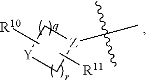

are as defined herein.

In certain embodiments, the subject compounds are of formula Ia.

In certain embodiments, the subject compounds are of formula Ib.

In certain embodiments of formula I, the subject compounds may be of formula IIa or IIb

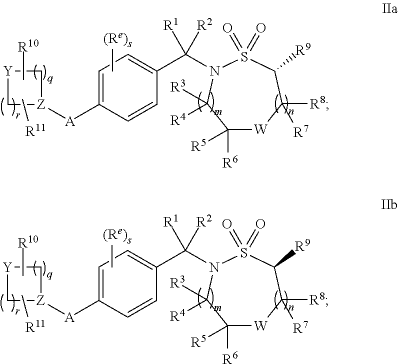

wherein s is from 0 to 3,
and m, n, q, r, A, W, Y, Z, R¹, R², R³, R⁴, R⁵, R⁶, R⁷, R⁸, R⁹, R¹⁰, R¹¹ and R^{e}, and the group

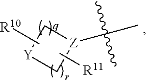

are as defined herein.

In certain embodiments, the subject compounds are of formula IIa.

In certain embodiments, the subject compounds are of formula IIb.

In certain embodiments of formula IIa or IIb, R^{e} is halo.

In certain embodiments of formula IIa or IIb, R^{e} is fluoro.

In certain embodiments of formula IIa or IIb, s is 0 or 1.

In certain embodiments of formula IIa or IIb, s is 0.

In certain embodiments of formula IIa or IIb, s is 1.

In certain embodiments of formula IIa or IIb, s is 1 or 2.

In certain embodiments of formula IIa or IIb, s is 2.

In certain embodiments of formula IIa or IIb, s is 1, 2 or 3.

In certain embodiments of formula IIa or IIb, s is 2 or 3.

In certain embodiments of formula IIa or IIb, s is 3.

In certain embodiments of formula I, the subject compounds may be of formula IIIa or IIIb:

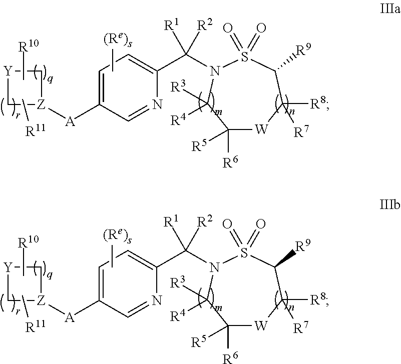

wherein m, n, q, r, s, A, W, Y, Z, R¹, R², R³, R⁴, R⁵, R⁶, R⁷, R⁸, R⁹, R¹⁰, R¹¹ and R^{e}, and the group

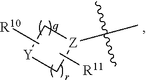

are as defined herein.

In certain embodiments, the subject compounds are of formula IIIa.

In certain embodiments, the subject compounds are of formula IIIb.

In certain embodiments of formula I, the subject compounds may be of formula IVa or IV

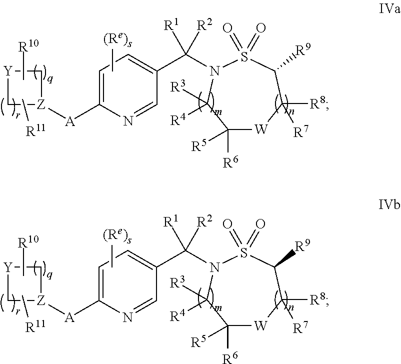

wherein m, n, q, r, s, A, W, Y, Z, R¹, R², R³, R⁴, R⁵, R⁶, R⁷, R⁸, R⁹, R¹⁰, R¹¹, and R^{e}, and the group

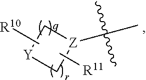

are as defined herein.

In certain embodiments, the subject compounds are of formula IVa.

In certain embodiments, the subject compounds are of formula IVb.

In certain embodiments of formula I, the subject compounds may be of formula Va or Vb:

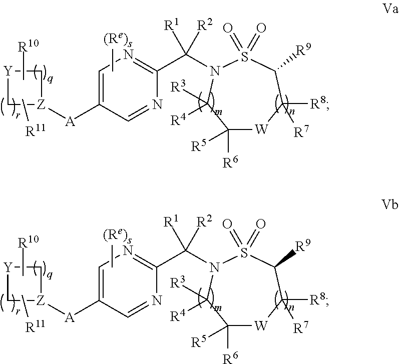

wherein m, n, q, r, s, A, W, Y, Z, R¹, R², R³, R⁴, R⁵, R⁶, R⁷, R⁸, R⁹, R¹⁰, R¹¹ and R^{e}, and the group

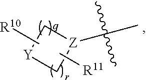

are as defined herein.

In certain embodiments, the subject compounds are of formula Va.

In certain embodiments, the subject compounds are of formula Vb.

In certain embodiments of formula I, the subject compounds may be of formula VIa or VIb:

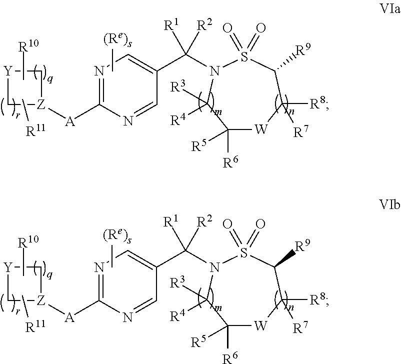

wherein m, n, q, r, s, A, W, Y, Z, R¹, R², R³, R⁴, R⁵, R⁶, R⁷, R⁸, R⁹, R¹⁰, R¹¹ and R^{e}, and the group

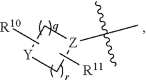

are as defined herein.

In certain embodiments, the subject compounds are of formula VIa.

In certain embodiments, the subject compounds are of formula VIb.

In certain embodiments of formula I, the subject compounds may be of formula VIIa or VIIb:

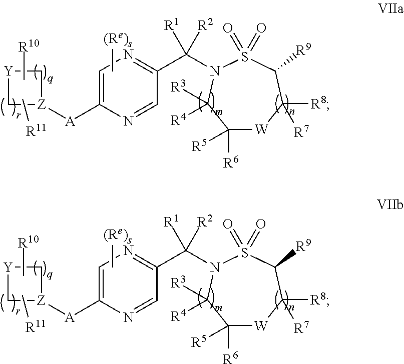

wherein m, n, q, r, s, A, W, Y, Z, R¹, R², R³, R⁴, R⁵, R⁶, R⁷, R⁸, R⁹, R¹⁰, R¹¹ and R^{e}, and the group

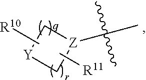

are as defined herein.

In certain embodiments, the subject compounds are of formula VIIa.

In certain embodiments, the subject compounds are of formula VIIb.

In certain embodiments of formula I, the subject compounds may be of formula VIIIa or VIIIb:

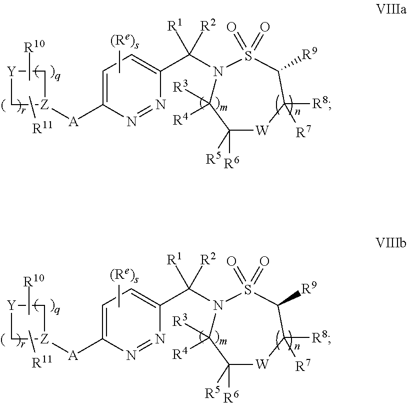

wherein m, n, q, r, s, A, W, Y, Z, R¹, R², R³, R⁴, R⁵, R⁶, R⁷, R⁸, R⁹, R¹⁰, R¹¹ and R^{e}, and the group

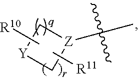

are as defined herein.

In certain embodiments, the subject compounds are of formula VIIIa.

In certain embodiments, the subject compounds are of formula VIIIb.

In certain embodiments of formula I, the subject compounds may be of formula IXa or IXb:

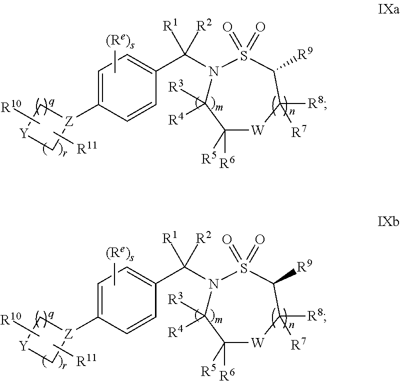

wherein m, n, q, r, s, W, Y, Z, R¹, R², R³, R⁴, R⁵, R⁶, R⁷, R⁸, R⁹, R¹⁰, R¹¹ and R^{e}, and the group

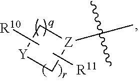

are as defined herein.

In certain embodiments, the subject compounds are of formula IXa.

In certain embodiments, the subject compounds are of formula IXb.

In certain embodiments of formula I, the subject compounds may be of formula Xa or Xb:

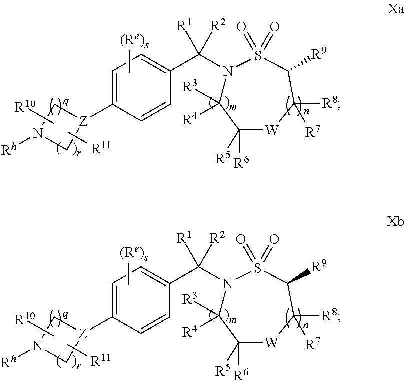

wherein m, n, q, r, s, W, Y, Z, R¹, R², R³, R⁴, R⁵, R⁶, R⁷, R⁸, R⁹, R¹⁰, R¹¹, R^{e} and R^{h} are as defined herein.

In certain embodiments, the subject compounds are of formula Xa.

In certain embodiments, the subject compounds are of formula Xb.

In certain embodiments of formula I, the subject compounds may be of formula XIa or XIb:

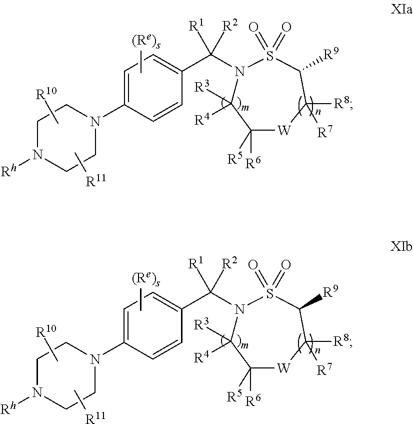

wherein m, n, s, W, R¹, R², R³, R⁴, R⁵, R⁶, R⁷, R⁸, R⁹, R¹⁰, R¹¹, R^{e} and R^{h} are as defined herein.

In certain embodiments, the subject compounds are of formula XIa.

In certain embodiments, the subject compounds are of formula XIb.

In certain embodiments of formula I, the subject compounds may be of formula XIIa or XIIb:

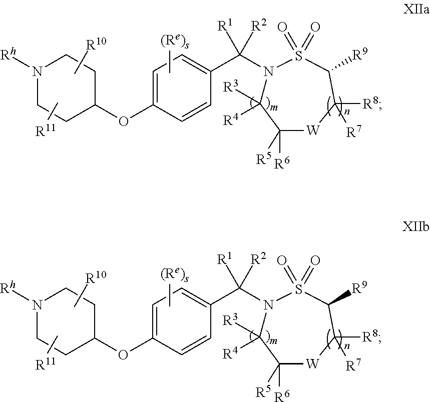

wherein m, n, s, W, R¹, R², R³, R⁴, R⁵, R⁶, R⁷, R⁸, R⁹, R¹⁰, R¹¹, R^{e} and R^{h} are as defined herein.

In certain embodiments, the subject compounds are of formula XIIa.

In certain embodiments, the subject compounds are of formula XIIb.

In certain embodiments of formula I, the subject compounds may be of formula XIIIa or XIIIb;

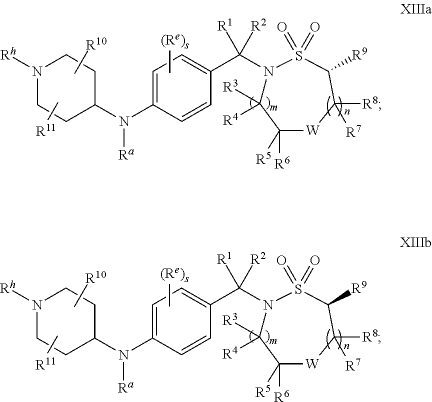

wherein m, n, p, s, W, R¹, R², R³, R⁴, R⁵, R⁶, R⁷, R⁸, R⁹, R¹⁰, R¹¹, R^{a}, R^{e} and R^{h} are as defined herein.

In certain embodiments, the subject compounds are of formula XIIIa.

In certain embodiments, the subject compounds are of formula XIIIb.

In certain embodiments of formula I, the subject compounds may be of formula XIVa or XIVb:

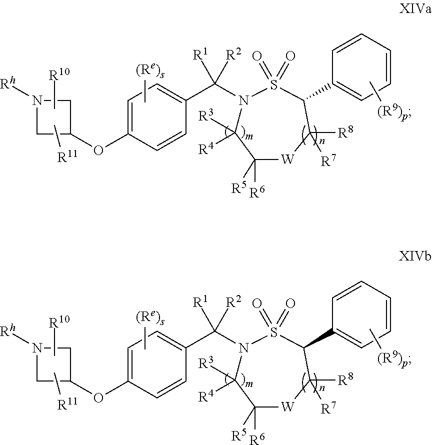

wherein m, n, s, W, R¹, R², R³, R⁴, R⁵, R⁶, R⁷, R⁸, R⁹, R¹⁰, R¹¹, R^{e} and R^{h} are as defined herein.

In certain embodiments, the subject compounds are of formula XIVa.

In certain embodiments, the subject compounds are of formula XIVb.

In certain embodiments of formula I, the subject compounds may be of formula XVa or XVb:

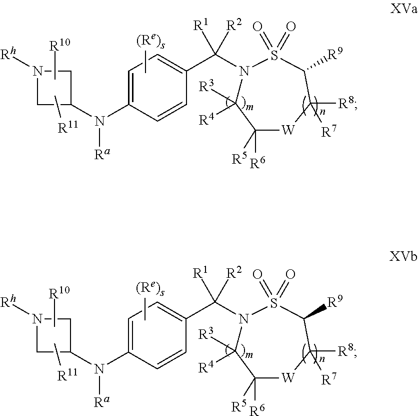

wherein m, n, s, W, R¹, R², R³, R⁴, R⁵, R⁶, R⁷, R⁸, R⁹, R¹⁰, R¹¹, R^{a}, R^{e} and R^{h} are as defined herein.

In certain embodiments, the subject compounds are of formula XVa.

In certain embodiments, the subject compounds are of formula XVb.

In certain embodiments of formula I, the subject compounds may be of formula XVIa or XVIb:

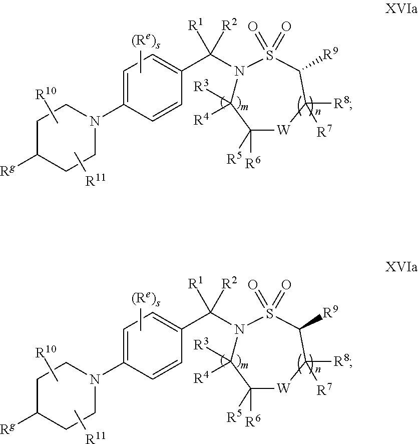

wherein m, n, p, s, W, R¹, R², R³, R⁴, R⁵, R⁶, R⁷, R⁸, R⁹, R¹⁰, R¹¹, R^{e} and R^{g} are as defined herein.

In certain embodiments, the subject compounds are of formula XVIa.

In certain embodiments, the subject compounds are of formula XVIb.

In certain embodiments of formula I, the subject compounds may be of formula XVIIa or XVIIb:

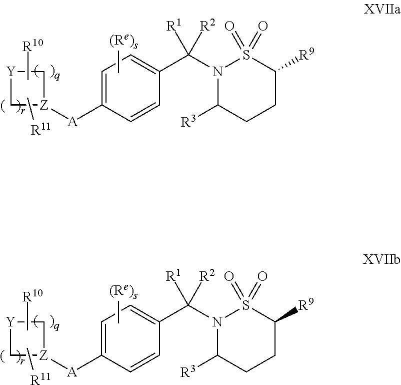

wherein q, r, s, A, Y, Z, R¹, R², R³, R⁹, R¹⁰, R¹¹ and R^{e}, and the group

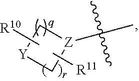

are as defined herein.

In certain embodiments, the subject compounds are of formula XVIIa.

In certain embodiments, the subject compounds are of formula XVIIb.

In certain embodiments of formula I, the subject compounds may be of formula XVIIIa or XVIIIb:

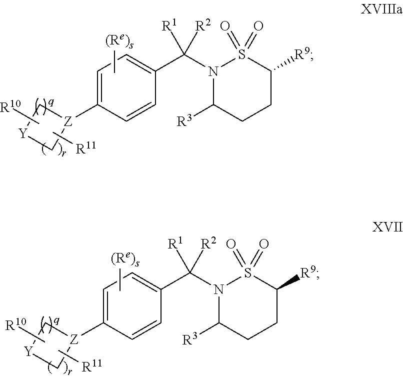

wherein q, r, s, Y, Z, R¹, R², R³, R⁹, R¹⁰, R¹¹ and R^{e}, and the group

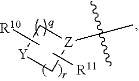

are as defined herein.

In certain embodiments, the subject compounds are of formula XVIIIa.

In certain embodiments, the subject compounds are of formula XVIIIb.

In certain embodiments of formula I, the subject compounds may be of formula XIXa or XIXb:

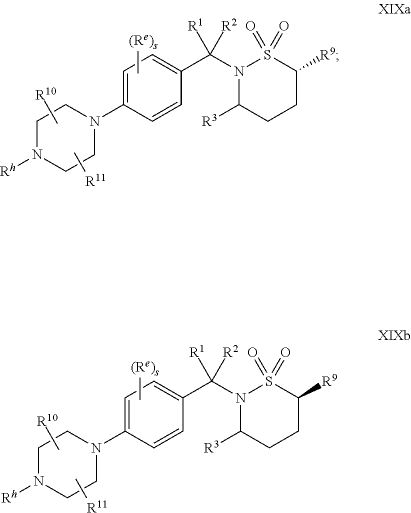

wherein m, n, s, R¹, R², R³, R⁹, R¹⁰, R¹¹, R^{e} and R^{h} are as defined herein.

In certain embodiments, the subject compounds are of formula XIXa.

In certain embodiments, the subject compounds are of formula XIXb.

In certain embodiments of formula I, the subject compounds may be of formula XXa or XXb:

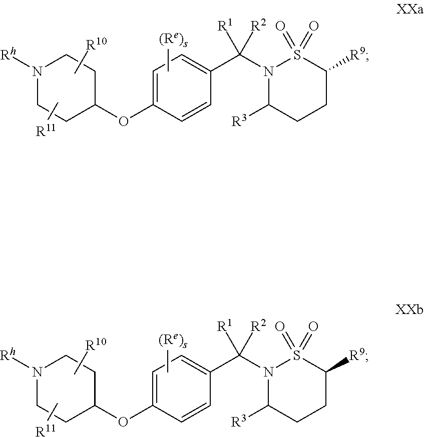

wherein m, n, s, R¹, R², R³, R⁹, R¹⁰, R¹¹, R^{e} and R^{h} are as defined herein.

In certain embodiments, the subject compounds are of formula XXa.

In certain embodiments, the subject compounds are of formula XXb.

In certain embodiments of formula I, the subject compounds may be of formula XXIa or XXIb;

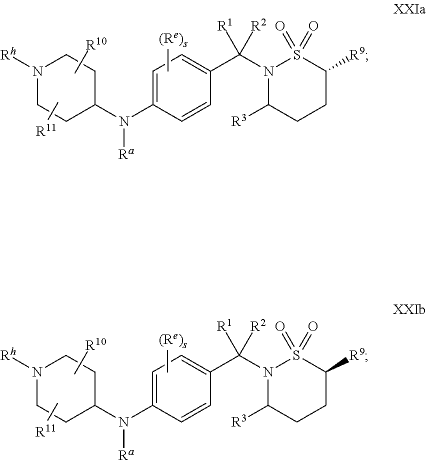

wherein m, n, s, R¹, R², R³, R⁹, R¹⁰, R¹¹, R^{a}, R^{e} and R^{h} are as defined herein.

In certain embodiments, the subject compounds are of formula XXIa.

In certain embodiments, the subject compounds are of formula XXIb.

In certain embodiments of formula I, the subject compounds may be of formula XXIIa or XXIIb:

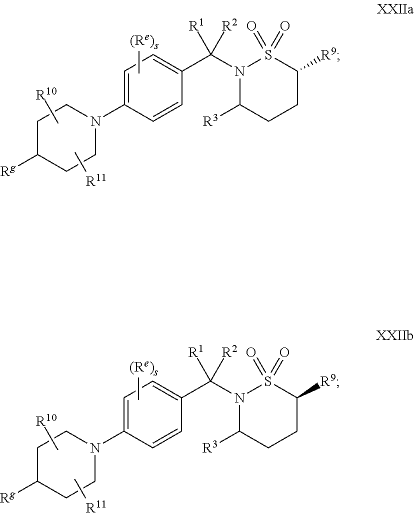

wherein m, n, s, R¹, R², R³, R⁹, R¹⁰, R¹¹, R^{e} and R^{g} are as defined herein.

In certain embodiments, the subject compounds are of formula XXIIa.

In certain embodiments, the subject compounds are of formula XXIIb.

In certain embodiments, the subject compounds may be of one of formulas XXIIIa through XIIId:

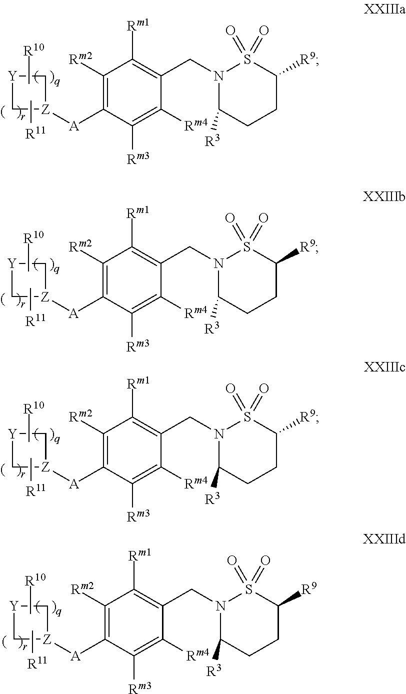

wherein R^{m1}, R^{m2}, R^{m3} and R^{m4} each independently is: hydrogen; or halo;
and q, r, A, Y, Z, R³, R⁹, R¹⁰, and R¹¹, and the group

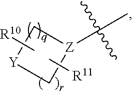

are as defined herein.

In certain embodiments, the subject compounds are of formula XXIIIa.

In certain embodiments, the subject compounds are of formula XXIIIb.

In certain embodiments, the subject compounds are of formula XXIIIc.

In certain embodiments, the subject compounds are of formula XXIIId.

In certain embodiments, R^{m1}, R^{m2}, R^{m3} and R^{m4} each independently is: hydrogen; or fluoro.

In certain embodiments, R^{m1} is fluoro and R^{m2}, R^{m3} and R^{m4} are hydrogen.

In certain embodiments, R^{m2} is fluoro and R^{m1}, R^{m3} and R^{m4} are hydrogen.

In certain embodiments, R^{m3} is fluoro and R^{m1}, R^{m2} and R^{m4} are hydrogen.

In certain embodiments, R^{m1} and R^{m2} are fluoro and R^{m3} and R^{m4} are hydrogen.

In certain embodiments, R^{m1} and R^{m3} are fluoro and R^{m2} and R^{m4} are hydrogen.

In certain embodiments, R^{m1} and R^{m4} are fluoro and R^{m2} and R^{m3} are hydrogen.

In certain embodiments, R^{m1}, R^{m2} and R^{m4} are fluoro and R^{m3} is hydrogen.

In certain embodiments, R^{m1}, R^{m2}, R^{m3} and R^{m4} are fluoro.

In certain embodiments, the subject compounds may be of one of formula XXIVa through XXIVd:

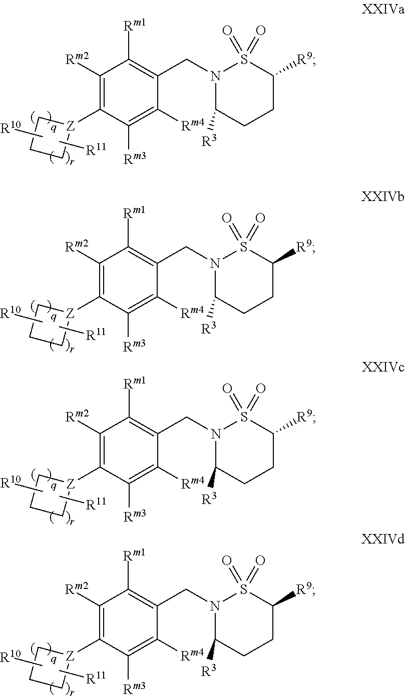

wherein q, r, Y, Z, R³, R⁹, R¹⁰, R¹¹, R^{m1}, R^{m2}, R^{m3} and R^{m4}, and the group

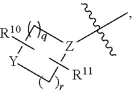

are as defined herein.

In certain embodiments, the subject compounds are of formula XXIXa.

In certain embodiments, the subject compounds are of formula XXIXb.

In certain embodiments, the subject compounds are of formula XXIXc.

In certain embodiments, the subject compounds are of formula XXIXd.

In certain embodiments of formula I, the subject compounds may be of one of formulas XXVa through XXVd:

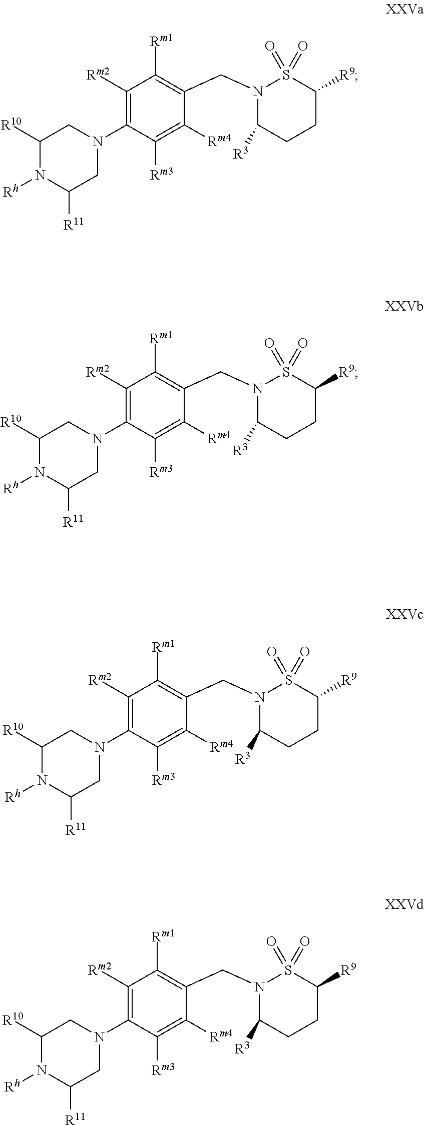

wherein R³, R⁹, R¹⁰, R¹¹, R^{h}, R^{m1}, R^{m2}, R^{m3} and R^{m4} are as defined herein.

In certain embodiments, the subject compounds are of formula XXVa.

In certain embodiments, the subject compounds are of formula XXVb.

In certain embodiments, the subject compounds are of formula XXVc.

In certain embodiments, the subject compounds are of formula XXVd.

In certain embodiments of formula I, the subject compounds may be of one of formulas XXVIa through XXVId:

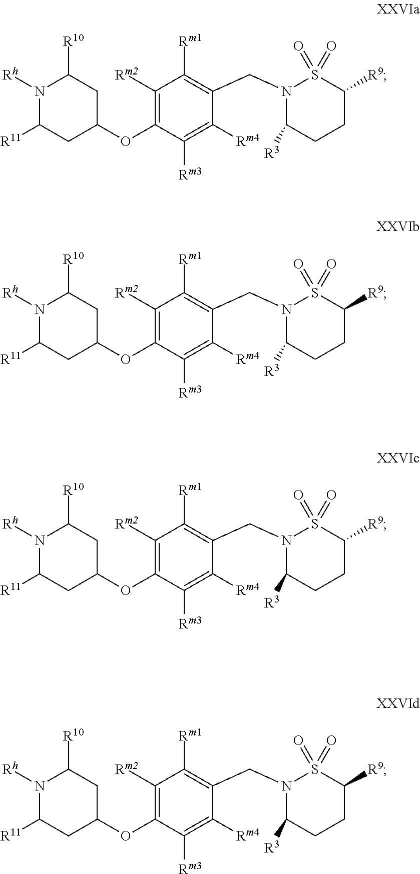

wherein R³, R⁹, R¹⁰, R¹¹, R^{h}, R^{m1}, R^{m2}, R^{m3} and R^{m4} are as defined herein.

In certain embodiments, the subject compounds are of formula XXVIa.

In certain embodiments, the subject compounds are of formula XXVIb.

In certain embodiments, the subject compounds are of formula XXVIc.

In certain embodiments, the subject compounds are of formula XXVId.

In certain embodiments of formula I, the subject compounds may be of one of formulas XXVIIa through XXVIId;

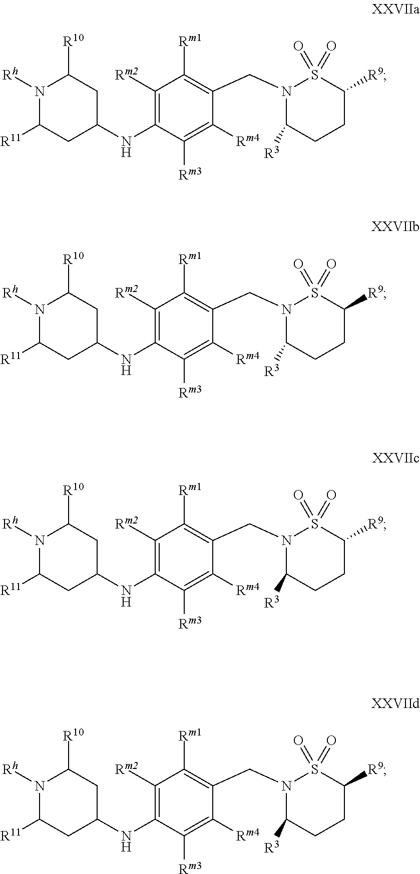

wherein R³, R⁹, R¹⁰, R¹¹, R^{h}, R^{m1}, R^{m2}, R^{m3} and R^{m4} are as defined herein.

In certain embodiments, the subject compounds are of formula XXVIIa.

In certain embodiments, the subject compounds are of formula XXVIIb.

In certain embodiments, the subject compounds are of formula XXVIIc.

In certain embodiments, the subject compounds are of formula XXVIId.

In certain embodiments of formula I, the subject compounds may be of one of formulas XXVIIIa through XXVIIId:

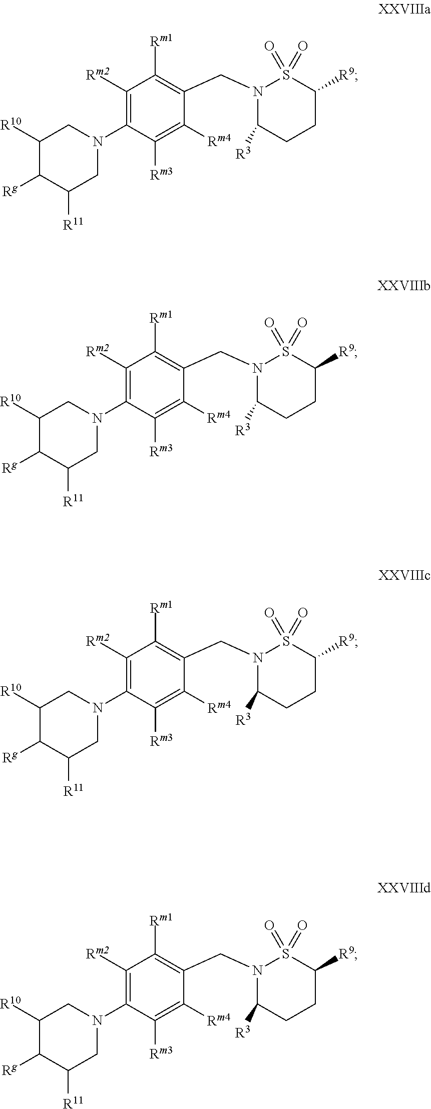

wherein R³, R⁹, R¹⁰, R¹¹, R^{h}, R^{m1}, R^{m2}, R^{m3} and R^{m4} are as defined herein.

In certain embodiments, the subject compounds are of formula XXVIIIa.

In certain embodiments, the subject compounds are of formula XXVIIIb.

In certain embodiments, the subject compounds are of formula XXVIIIc.

In certain embodiments, the subject compounds are of formula XXVIIId.

In certain embodiments of formula I, the subject compounds may be one of formulas XXIXIa through XXIXd:

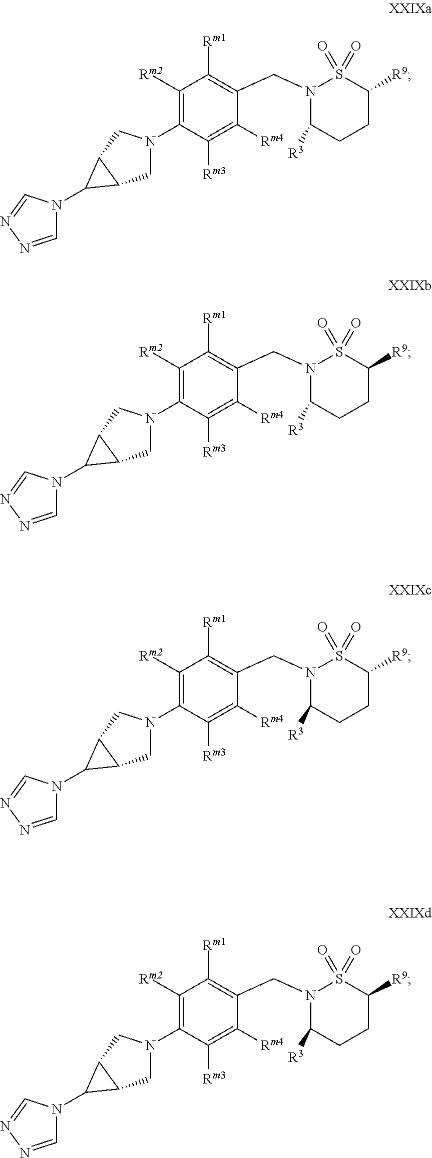

wherein:

R⁹ is: hydrogen; C₁₋₆alkyl; C₃-cycloalkyl; heterocyclyl; or heteroaryl; each of which may be unsubstituted or substituted one or more times with Rⁱ; and

R³, R¹⁰, R¹¹, R^{h}, R^{m1}, R^{m2}, R^{m3} and R^{m4} are as defined herein.

Methods

The invention also provides a method for treating a disease or condition mediated by or otherwise associated with the RORc receptor, the method comprising administering to a subject in need thereof an effective amount of a compound of the invention.

The disease may be arthritis such as rheumatoid arthritis or osteoarthritis.

The disease may be asthma or COPD.

Representative compounds in accordance with the methods of the invention are shown in the experimental examples below.

Synthesis

Compounds of the present invention can be made by a variety of methods depicted in the illustrative synthetic reaction schemes shown and described below.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein may be conducted under an inert atmosphere at atmospheric pressure at a reaction temperature range of from about −78° C. to about 150° C., for example, from about 0° C. to about 125° C., or conveniently at about room (or ambient) temperature, e.g., about 20° C.

Scheme A below illustrates one synthetic procedure usable to prepare specific compounds of formula I, wherein LG is a leaving group such as halo and may be the same or different at each occurrence, and m, n, q, A, X¹, X³, X⁴, R¹, R², R³, R⁴, R⁵, R⁶, R⁷, R⁸, R⁹, R¹⁰, R^{b} and R^{c} are as defined herein.

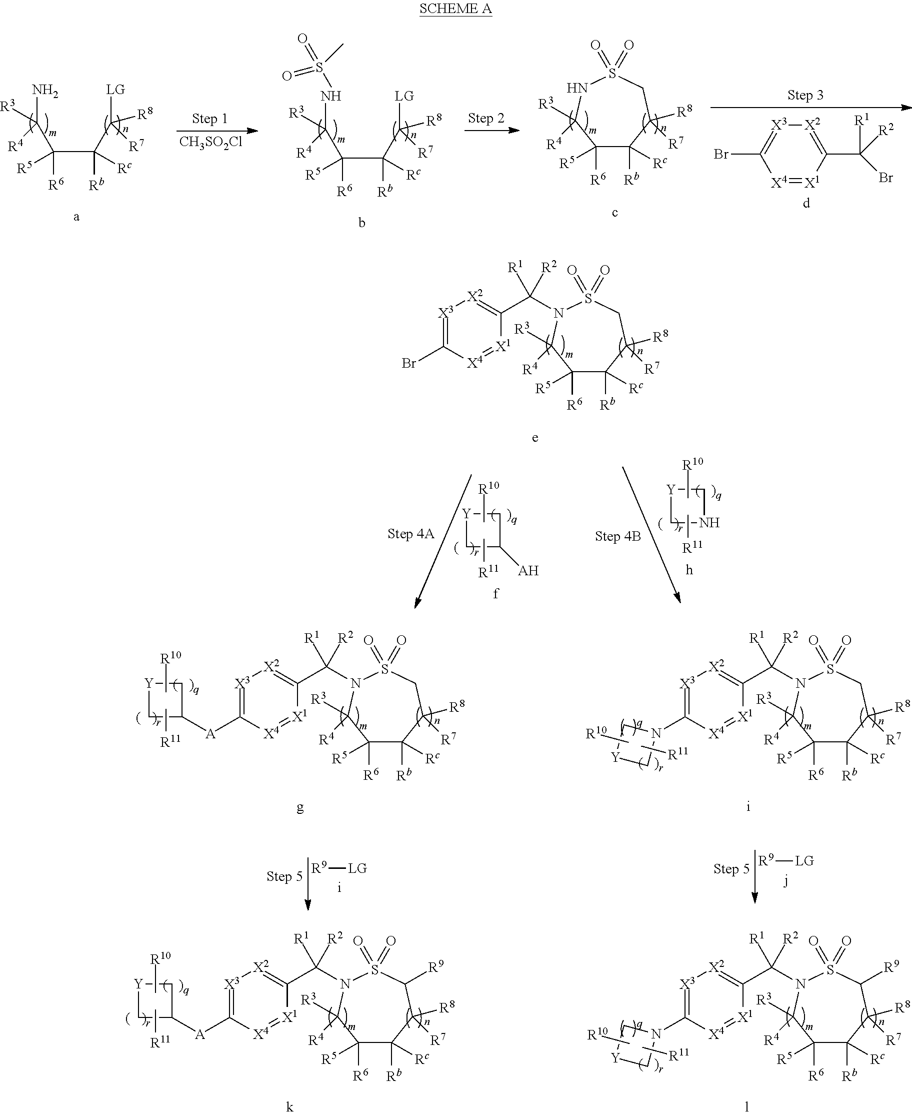

In step 1 of Scheme A, alkyl amine a is reacted with methanesulfonyl chlorided to form sulfonamide compound b. The reaction of step 1 may be carried out in a polar aprotic solvent such as THF, and in the presence of a tertiary amine base. The leaving group of compound a may be bromo or chloro in certain embodiments.

A cyclization reaction is carried out in step 2 to afford thiazinane compound c. The cyclization may be achieved in the presence of a strong base such as an alkyl lithium reagent, using polar aprotic solvent under anhydrous conditions.

In step 3, thiazinane compound c is reacted with aryalkyl halide compound d to yield aralkyl thiazinane e. The reaction of step 3 may be carried out in the presence of a strong base such as sodium hydride under anhydrous polar aprotic solvent conditions. The bromo groups of compound e may be replaced by other suitable leaving groups used in the art.

Thiazinane compound e may be treated with reagent fin step 4A to provide sultam compound g. In embodiments wherein A is oxygen such that reagent g is a cyclic alcohol, the reaction of step 4A may utilize a copper catalyst with hydrophobic solvent, in the presence of cesium carbonate or like base.

Alternatively, step 4B may be carried out wherein thiazinane compound e undergoes amination by reaction with cyclic amine h to afford sultam compound j, which is a compound of formula I in accordance with the invention. The reaction of step may utilize a suitable palladium catalyst under Buchwald reaction conditions.

In step 5, sultam compound g or i is treated with alkylating reagent j to afford sultam k or l respectively, which are compounds of formula I in accordance with the invention. In many embodiments reagent j is a heteroaryl halide such as thienyl iodide. The reaction of step 5 may be carried out in the presence of a suitable palladium catalyst in polar aprotic solvent such as THF.

Scheme B below shows another synthetic procedure usable to prepare specific compounds of formula I, wherein m, n, q, A, X¹, X², X³, X⁴, Y, R¹, R², R³, R⁴, R⁵, R⁶, R⁹ and R¹⁰ are as defined herein.

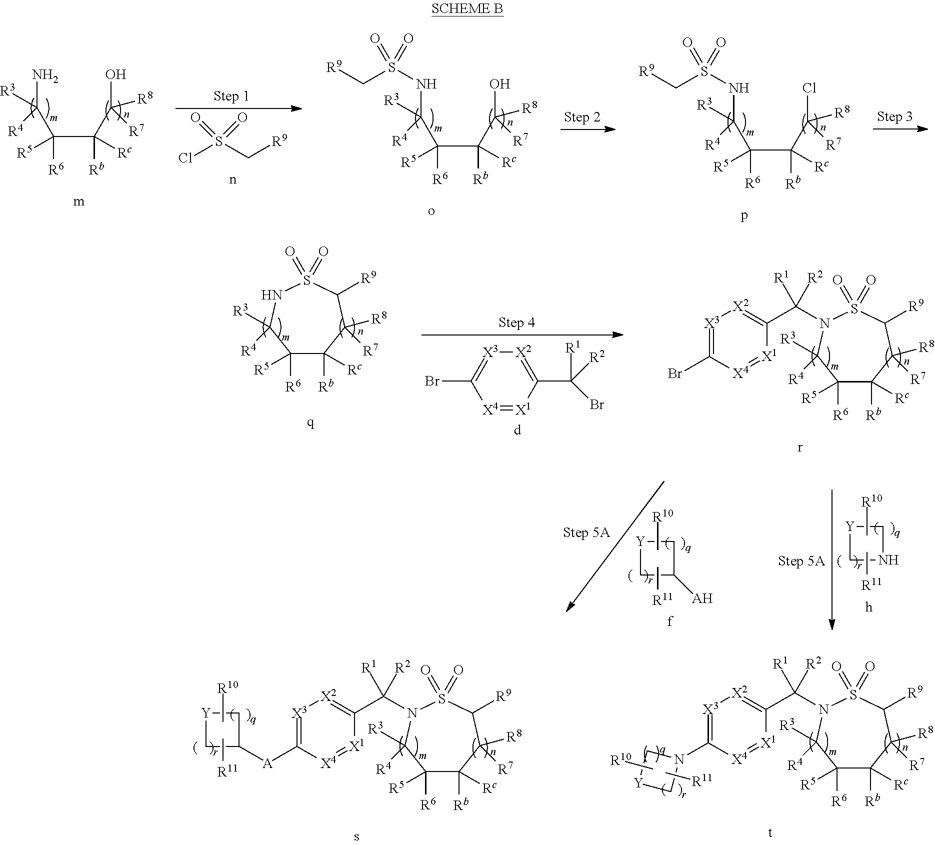

In step 1 of Scheme B, hydroxy-alkyl amine compound m is reacted with sulfonyl halide reagent n to form hydroxy-alkyl sulfonamide compound o.

In step 2, hydroxy-alkyl sulfonamide compound o is reacted with an alkalai metal halide salt (not shown) such as NaCl to provide halo-alkyl sulfonamide compound p.

In step 3, a cyclization reaction is affected to provide thiazinane compound q. The cyclization may be carried out in the presence of alkyllithium reagent under polar aprotic solvent conditions.

In step 4, thiazinane compound q is reacted with aryalkyl halide compound d to yield aralkyl thiazinane r. The reaction of step 4 may be carried out in the presence of a strong base such as sodium hydride under anhydrous polar aprotic solvent conditions. The bromo groups of compound d may be replaced by other suitable leaving groups as described above.

Steps 5A or 5B may then be carried out by reaction of aralkyl thiazinane r with reagents f and h respectively, in the manner described above with reference to Scheme A, to afford sultam compounds s and t respectively, which are compounds of formula I in accordance with the invention.

Many variations on the procedures of Scheme A and Scheme B are possible and will suggest themselves to those skilled in the art. Specific details for producing compounds of the invention are described in the Examples below.

Administration and Pharmaceutical Composition

The invention includes pharmaceutical compositions comprising at least one compound of the present invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients.

In general, the compounds of the invention will be administered in a therapeutically effective amount by any of the accepted modes of administration for agents that serve similar utilities. Suitable dosage ranges are typically 1-500 mg daily, for example 1-100 mg daily, and most preferably 1-30 mg daily, depending upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this Application, to ascertain a therapeutically effective amount of the compounds of the present invention for a given disease.

Compounds of the invention may be administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation. A particular manner of administration is generally oral using a convenient daily dosage regimen which can be adjusted according to the degree of affliction.

A compound or compounds of the invention, together with one or more conventional adjuvants, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

The compounds of the invention may be formulated in a wide variety of oral administration dosage forms. The pharmaceutical compositions and dosage forms may comprise a compound or compounds of the present invention or pharmaceutically acceptable salts thereof as the active component. The pharmaceutically acceptable carriers may be either solid or liquid. Solid form preparations include powders, tablets, pills, capsules, cachets, suppositories, and dispersible granules. A solid carrier may be one or more substances which may also act as diluents, flavouring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets may contain from about one (1) to about seventy (70) percent of the active compound. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The compounds of the invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of the invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

The compounds of the invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the invention may be formulated for vaginal administration. Pessaries, tampons, creams, gels, pastes, foams or sprays containing in addition to the active ingredient such carriers as are known in the art to be appropriate.

The subject compounds may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatine or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylazacycloheptan-2-one). Sustained release delivery systems are inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations may be in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa. Representative pharmaceutical formulations containing a compound of the present invention are described below.

Utility

The compounds of the invention are useful for treatment of immune disorders generally. The compounds may be used for treatment of arthritis, including rheumatoid arthritis, osteoarthritis, psoriatic arthritis, septic arthritis, spondyloarthropathies, gouty arthritis, systemic lupus erythematosus and juvenile arthritis, osteoarthritis, and other arthritic conditions.

The compounds may be used for treatment of respiratory disorders such as chronic obstructive pulmonary disease (COPD), asthma, bronchospasm, and the like.

The compounds may be used for treatment of gastrointestinal disorder (“GI disorder”) such as Irritable Bowel Syndrome (IBS), Inflammatory Bowel Disease (IBD), biliary colic and other biliary disorders, renal colic, diarrhea-dominant IBS, pain associated with GI distension, and the like.

The compounds may be used for treatment of pain conditions such as inflammatory pain; arthritic pain, surgical pain; visceral pain; dental pain; premenstrual pain; central pain; pain due to burns; migraine or cluster headaches; nerve injury; neuritis; neuralgias; poisoning; ischemic injury; interstitial cystitis; cancer pain; viral, parasitic or bacterial infection; post-traumatic injury; or pain associated with irritable bowel syndrome.

### GENERAL EXPERIMENTAL

### LCMS Methods

High Pressure Liquid Chromatography-Mass Spectrometry (LCMS) experiments to determine retention times (RT) and associated mass ions were performed using one of the following methods:

Method A: Compounds were analysed using the following conditions: Experiments were performed on a Waters ZMD single quadrupole mass spectrometer linked to a Hewlett Packard HP1100 LC system with UV diode array detector and 100 position autosampler. The spectrometer has an electrospray source operating in positive and negative ion mode. This system uses a Phenomenex Luna 3 μm C18(2) 30×4.6 mm column at ambient temperature and a 2.0 mL/minute flow rate. The initial solvent system was 95% water containing 0.1% formic acid (solvent A) and 5% acetonitrile containing 0.1% formic acid (solvent B) for the first 0.5 minute followed by a gradient up to 5% solvent A and 95% solvent B over the next 4 minutes. This was maintained for 1 minute before returning to 95% solvent A and 5% solvent B over the next 0.5 minute. Total run time was 6 minutes.

Method B: Compounds were analysed using the following conditions: Experiments were performed on a Waters Micromass ZQ2000 quadrupole mass spectrometer linked to a Waters Acquity UPLC system with a PDA UV detector. The spectrometer has an electrospray source operating in positive and negative ion mode. This system uses an Acquity BEH C18 1.7 μm 100×2.1 mm column, maintained at 40° C. or an Acquity BEH Shield RP18 1.7 μm 100×2.1 mm column, maintained at 40° C. and a 0.4 mL/minute flow rate. The initial solvent system was 95% water containing 0.1% formic acid (solvent A) and 5% acetonitrile containing 0.1% formic acid (solvent B) for the first 0.4 minute followed by a gradient up to 5% solvent A and 95% solvent B over the next 5.6 minutes. This was maintained for 0.8 minute before returning to 95% solvent A and 5% solvent B over the next 1.2 minutes. Total run time was 8 minutes.

### NMR Methods:

¹H NMR spectra were recorded at ambient temperature or at 80° C. where indicated using one of the following machines: Varian Unity Inova (400 MHz) spectrometer with a triple resonance 5 mm probe, Bruker Avance DRX 400 (400 MHz) spectrometer with a triple resonance 5 mm probe, a Bruker Avance DPX 300 (300 MHz) equipped with a standard 5 mm dual frequency probe for detection of 1H and 13C, Bruker Fourier 300 MHz system equipped with a standard 5 mm 1H/13C probe, a Bruker AVIII (400 MHz) using a BBI Broad Band Inverse 5 mm probe, or a Bruker AVIII (500 MHz) using a QNP (Quad Nucleus detect) 5 mm probe. Chemical shifts are expressed in ppm relative to an internal standard, tetramethylsilane (ppm=0.00). The following abbreviations have been used: br=broad signal, s=singlet, d=doublet, dd=double doublet, t=triplet, td=triplet doublet, dddd=doublet doublet doublet doublet, q=quartet, m=multiplet, or any combination of.

### Microwave Reactor:

Microwave reactions were carried out using a Biotage® Initiator® in vials appropriate to the scale of the reaction and at the temperature and time described in the experimental details.

### Purification Equipment:

Purifications were carried out using pre-packed silica gel cartridges either on a Teledyne ISCO CombiFlash® or Biotage® Isolera Four® or using compressed air to apply external pressure. Solvents and gradients shown in the experimental details were used.

Reverse Phase High Pressure Liquid Chromatography (HPLC) was used to purify compounds where indicated. Separation using gradient elution on a Phenomenex Gemini C18 column (250×21.2 mm, 5 micron) as stationary phase and using mobile phase indicated, operating at a 18 mL/min flow rate using a Gilson UV/Vis-155 dual channel detector and Gilson GX-271 automated liquid handler.

Phase separator cartridges are supplied by Biotage® as Isolute® phase separator cartridges.

### LIST OF ABBREVIATIONS

AcOH Acetic acid
AIBN 2,2′-Azobis(2-methylpropionitrile)
Atm. Atmosphere
BOC tert-Butyloxycarbonyl group
(BOC)₂O Di-tert-butyl dicarbonate
CrO₃ Chromium (VI) oxide
CDCl₃ Deuterated chloroform
DavePhos 2-Dicyclohexylphosphino-2′-(N,N-dimethylamino)biphenyl
DCM Dichloromethane/methylene chloride
DMA N,N-Dimethylacetamide
DIAD Diisopropyl azodicarboxylate
DIPEA DIPEA
DMAP 4-Dimethylaminopyridine
DME 1,2-Dimethoxyethane
DMF N,N-Dimethylformamide
DMSO Dimethyl sulfoxide
DPPF 1,1′-Bis(diphenylphosphino)ferrocene
ES Electrospray
Et₂O Diethyl ether
Et₃N Triethylamine
EtOH Ethanol/Ethyl alcohol
EtOAc Ethyl acetate
H₂O Water
H₂SO₄ Sulfuric acid
HATU 2-(1H-7-Azabenzotriazol-1-yl)-1, 1,3,3-tetramethyl uronium hexafluorophosphate methanaminium
HBTU O-Benzotriazol-1-yl-N,N,N′,N′-tetramethyluronium hexafluorophosphate
HCO₂H Formic acid
HCl Hydrochloric acid
HOBT 1-Hydroxybenzotriazole
HPLC High pressure liquid chromatography
RP HPLC Reverse phase high pressure liquid chromatography
IBX 2-Iodoxybenzoic acid
IMS Industrial methylated spirit
KOH Potassium hydroxide
K₂CO₃ Potassium carbonate
LDA Lithium diisopropylamide
i-PrOH Isopropanol/isopropyl alcohol/propan-2-ol
LCMS Liquid Chromatograph/Mass Spectroscopy
LiOH Lithium hydroxide
MgSO₄ Magnesium sulphate
MeOH Methanol/Methyl alcohol
MW Microwaves
NaH Sodium hydride
NaCl Sodium chloride
NaOH Sodium hydroxide
Na₂SO₄ Sodium sulfate
Na₂CO₃ Sodium carbonate
NaHCO₃ Sodium bicarbonate/Sodium hydrogen carbonate
NBS N-Bromosuccinimide
NH₄Cl Ammonium chloride
NMP 1-Methyl-2-pyrrolidinone
POCl₃ Phosphorus oxychloride
PhCH₃ Toluene
Pd₂(dba)₃ Tris(dibenzylideneacetone)dipalladium (0)
PSI Pound per square inch
RT Room temperature
sat. Saturated
SCX-2 Pre-packed Isolute® silica-based sorbent with a chemically bonded propylsulfonic acid functional group
TBDMS tert-Butyldimethylsilyl
TFA Trifluoroacetic acid
THF Tetrahydrofuran
TIPS Triisopropylsilyl
TLC Thin layer chromatography
XantPhos 4,5-Bis(diphenylphosphino)-9,9-dimethylxanthene

### Example 1

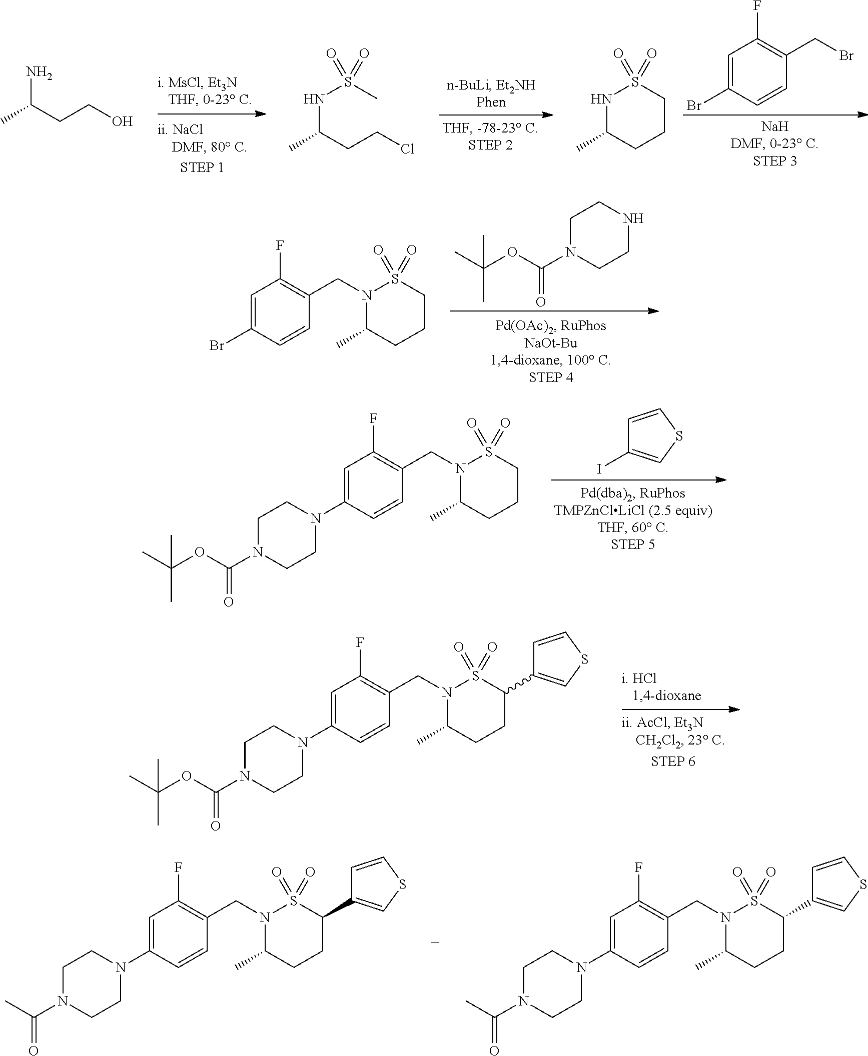

### Step 1

### N-[(1S)-3-chloro-1-methyl-propyl]methanesulfonamide

To a solution of (3S)-3-aminobutan-1-ol (3.0 g, 34 mmol) and triethylamine (12 mL, 84 mmol) in tetrahydrofuran (100 mL) at 0° C. was slowly added methanesulfonyl chloride (6.6 mL, 84 mmol). The reaction was then allowed to worm to room temperature and was stirred at that temperature for 16 hours. Methyl tert-butyl ether (100 mL) was then added to precipitate triethylamine hydrochloride and the salt was removed by filtration. The filtrate was then concentrated to give crude bis-sulfonylated intermediate.

To the crude intermediate was added sodium chloride (4.9 g, 84 mmol) and N,N-dimethylformamide (100 mL) and the reaction was stirred at 80° C. for 16 hours. The reaction was then diluted with ethyl acetate (500 mL), washed with water and brine, dried with anhydrous MgSO₄, concentrated and purified by silica gel column chromatography (0-70% ethyl acetate in heptane) to give N-[(1S)-3-chloro-1-methyl-propyl]methanesulfonamide (2.41 g, 13.0 mmol, 39% yield). NMR (400 MHz, CDCl₃) δ 4.99-4.77 (d, J=9.0 Hz, 1H), 3.82-3.52 (m, 3H), 3.06-2.95 (s, 3H), 1.99-1.86 (m, 2H), 1.37-1.27 (d, J=6.6 Hz, 3H); LCMS (m/z) ES⁺186 [M+1]⁺.

### Step 2

### (3S)-3-methylthiazinane 1,1-dioxide

To a solution of N-[(1S)-3-chloro-1-methyl-propyl]methanesulfonamide (2.41 g, 13.0 mmol) in tetrahydrofuran (43 mL) was added 1,10-phenanthroline (5.9 mg, 0.0325 mmol) and diisopropylamine (0.46 mL, 3.25 mmol) and the solution was cooled to −78° C. N-Butyllithium (2.5 M in hexanes, 18 mL, 45.4 mmol) was then added dropwise over 5 minutes. The reaction was allowed to warm to room temperature and was stirred at that temperature for 16 hours. The reaction was quenched with saturated NH₄Cl (aq) (10 mL), extracted with dichloromethane, dried with anhydrous MgSO₄ and concentrated to give (3S)-3-methylthiazinane 1,1-dioxide (2.1 g, 14 mmol, 110% yield). The product was used without further purification. LCMS (m/z) ES⁺150 [M+1]⁺.

### Step 3

### (3S)-2-[(4-bromo-2-fluoro-phenyl)methyl]-3-methyl-thiazinane 1,1-dioxide

To a solution of (3S)-3-methylthiazinane 1,1-dioxide (2.1 g, 14 mmol) and 4-bromo-1-(bromomethyl)-2-fluoro-benzene (5.28 g, 19.7 mmol) in N,N-dimethylacetamide (60 mL) at 0° C. was added sodium hydride (60% in mineral oil, 788 mg, 19.7 mmol) and the reaction was allowed to warm to room temperature and stirred at that temperature for 2 hours. Water (10 mL) was then added and the reaction was diluted with ethyl acetate (350 mL), washed with water and brine, dried with anhydrous MgSO₄, concentrated and purified by silica gel column chromatography (0-70% ethyl acetate in heptane) to give (3S)-2[(4-bromo-2-fluoro-phenyl)methyl]-3-methyl-thiazinane 1,1-dioxide (1.55 g, 4.62 mmol, 33% yield). LCMS (m/z) ES⁺336 [M+1]⁺.

### Step 4

### Tert-butyl 4-[3-fluoro-4-[[(3S)-3-methyl-1,1-dioxo-thiazinan-2-yl]methyl]phenyl]piperazine-1-carboxylate

A 20-mL vial was charged with (3S)-2[(4-bromo-2-fluoro-phenyl)methyl]-3-methyl-thiazinane 1,1-dioxide (526 mg, 1.56 mmol), tert-butyl piperazine-1-carboxylate (437 mg, 2.34 mmol), palladium(II) acetate (18 mg, 0.078 mmol), 2-dicyclohexylphosphino-2′,6′-di-i-propoxy-1,1′-biphenyl (74 mg, 0.15 mmol) and sodium tert-butoxide (228 mg, 2.34 mmol) and the vial was purged with nitrogen for 1 minutes. 1,4-Dioxane (8 mL) was then added and the reaction was stirred at 100° C. for 2 hours.

The mixture was diluted with dichloromethane (20 mL) and filtered through celite. The filtrate was concentrated and purified by silica gel column chromatography (0-100% ethyl acetate in heptane) to give tert-butyl 4-[3-fluoro-4-[[(3S)-3-methyl-1,1-dioxo-thiazinan-2-yl]methyl]phenyl]piperazine-1-carboxylate (610 mg, 1.38 mmol, 88% yield). ¹H NMR (400 MHz, DMSO) δ 7.32-7.25 (m, 1H), 6.81-6.76 (m, 1H), 6.75-6.68 (m, 1H), 4.27-4.19 (m, 1H), 4.19-4.12 (m, 1H), 3.85-3.74 (m, 1H), 3.48-3.40 (m, 4H), 3.17-3.01 (m, 6H), 2.13-2.03 (m, 1H), 2.03-1.91 (m, 1H), 1.59-1.47 (m, 2H), 1.43-1.39 (s, 9H), 1.12-1.05 (d, J=6.9 Hz, 3H); LCMS (m/z) ES⁺442 [M+1]⁺.

### Step 5

### Tert-butyl 4-[3-fluoro-4-[[(3S)-3-methyl-1, 1-dioxo-6-(3-thienyl)thiazinan-2-yl]methyl]phenyl]piperazine-1-carboxylate

A vial was charged with tert-butyl 4-[3-fluoro-4-[[(3S)-3-methyl-1,1-dioxo-thiazinan-2-yl]methyl]phenyl]piperazine-1-carboxylate (68 mg, 0.15 mmol), 3-iodothiophene (36 mg, 0.17 mmol), bis(dibenzylideneacetone)palladium (8.9 mg, 0.015 mmol) and 2-dicyclohexylphosphino-2′,6′-di-i-propoxy-1,1′-biphenyl (7.3 mg, 0.015 mmol) and the vial was purged with nitrogen. Tetrahydrofuran (1.5 mL) and 2,2,6,6-tetramethylpiperidinylzinc chloride lithium chloride complex (0.65 M in THF, 0.59 mL, 0.39 mmol) were then added and the reaction was stirred at 60° C. for 16 hours. The reaction was quenched with saturated NH₄Cl_{(aq)} (1 mL), extracted with ethyl acetate (2×2 mL), concentrated and purified by reverse-phase preparative HPLC to give tert-butyl 4-[3-fluoro-4[[(3S)-3-methyl-1,1-dioxo-6-(3-thienyl)thiazinan-2-yl]methyl]phenyl]piperazine-1-carboxylate (30 mg, 0.057 mmol, 38% yield). LCMS (m/z) ES⁺524 [M+1]⁺.

### Step 6

### 1-[4-[3-fluoro-4-[[(3S)-3-methyl-1,1-dioxo-6-(3-thienyl)thiazinan-2-yl]methyl]phenyl]piperazin-1-yl]ethanone

To a solution of tert-butyl 4-[3-fluoro-4-[[(3S)-3-methyl-1,1-dioxo-6-(3-thienyl)thiazinan-2-yl]methyl]phenyl]piperazine-1-carboxylate (30 mg, 0.057 mmol) in 1,4-dioxane (2 mL) at room temperature was added HCl (4N in 1,4-dioxane, 2 mL) and the reaction was stirred at that temperature for 2 hours. The reaction was then concentrated under vacuum to give crude BOC-deprotected intermediate.

To a solution of the crude intermediate in dichloromethane (2 mL) was added triethylamine (0.1 mL, 0.77 mmol), followed by acetyl chloride (0.055 mL, 0.77 mmol) and the reaction was stirred at room temperature for 1 hour. The reaction was then quenched with water (2 mL), extracted with extracted with dichloromethane (2×2 mL), concentrated and purified by reverse-phase preparative HPLC, followed by chiral supercritical fluid chromatography to give 1-[4-[3-fluoro-4-[[(3S)-3-methyl-1,1-dioxo-6-(3-thienyl)thiazinan-2-yl]methyl]phenyl]piperazin-1-yl]ethanone (ISOMER A) (7.6 mg, 0.016 mmol, 28% yield) and 1-[4-[3-fluoro-4-[[(3S)-3-methyl-1,1-dioxo-6-(3-thienyl)thiazinan-2-yl]methyl]phenyl]piperazin-1-yl]ethanone (ISOMER B) (5.2 mg, 0.011 mmol, 19% yield).

1-[4-[3-fluoro-4-[[(3S)-3-methyl-1,1-dioxo-6-(3-thienyl)thiazinan-2-yl]methyl]phenyl]piperazin-1-yl]ethanone (ISOMER A): ¹H NMR (400 MHz, DMSO) δ 7.59-7.52 (m, 2H), 7.38-7.28 (m, 1H), 7.22-7.15 (m, 1H), 6.83-6.78 (m, 1H), 6.76-6.69 (m, 1H), 4.64-4.54 (m, 1H), 4.43-4.33 (m, 1H), 4.30-4.22 (m, 1H), 4.11-3.98 (m, 1H), 3.59-3.52 (m, 4H), 3.22-3.15 (m, 2H), 3.15-3.09 (m, 2H), 2.42-2.29 (m, 1H), 2.20-2.09 (m, 1H), 2.07-2.00 (s, 3H), 1.84-1.69 (m, 1H), 1.68-1.58 (m, 1H), 1.12-1.03 (d, J=6.9 Hz, 3H); LCMS (m/z) ES⁺466.2 [M+1]⁺.

1-[4-[3-fluoro-4-[[(3S)-3-methyl-1,1-dioxo-6-(3-thienyl)thiazinan-2-yl]methyl]phenyl]piperazin-1-yl]ethanone (ISOMER B): ¹H NMR (400 MHz, DMSO) δ 7.61-7.57 (m, 1H), 7.57-7.53 (m, 1H), 7.31-7.24 (m, 1H), 7.22-7.19 (m, 1H), 6.83-6.74 (m, 2H), 4.54-4.46 (m, 1H), 4.37-4.31 (m, 2H), 3.59-3.51 (m, 5H), 3.25-3.20 (m, 2H), 3.18-3.13 (m, 2H), 2.69-2.57 (m, 1H), 2.16-1.99 (m, 5H), 1.64-1.55 (m, 1H), 1.35-1.28 (d, J=7.1 Hz, 3H); LCMS (m/z) ES⁺466.2 [M+1]⁺.

### Example 2

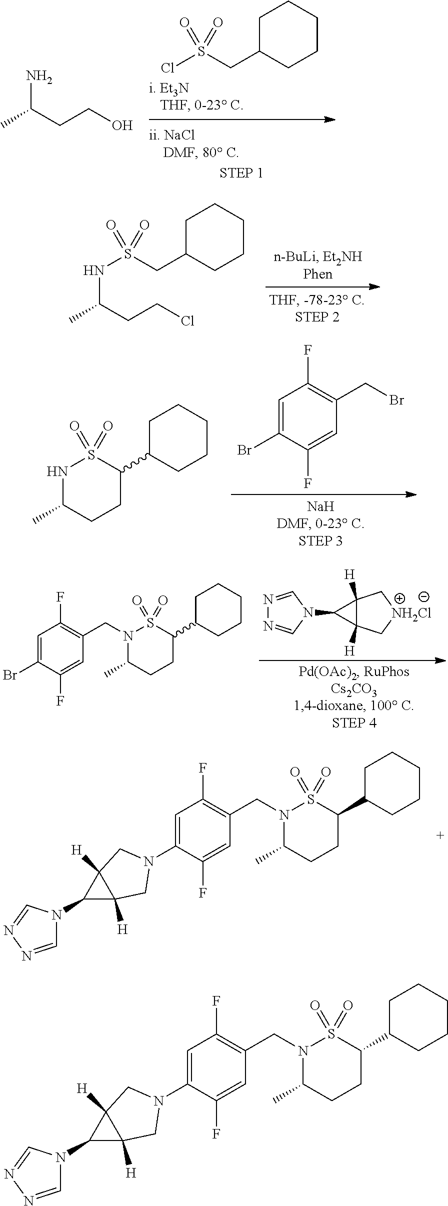

### Step 1

### N-[(1S)-3-chloro-1-methyl-propyl]-1-cyclohexyl-methane sulfonamide

To a solution of (3S)-3-aminobutan-1-ol (200 mg, 2.24 mmol) and triethylamine (0.79 mL, 5.61 mmol) in tetrahydrofuran (7 mL) at 0° C. was slowly added cyclohexylmethanesulfonyl chloride (1.0 g, 5.16 mmol) and the reaction was stirred at room temperature for 16 hours. Methyl tert-butyl ether (20 mL) was then added to precipitate triethylamine hydrochloride and the salt was removed by filtration. The filtrate was then concentrated to give crude bis-sulfonylated intermediate.

To the crude intermediate was added sodium chloride (328 mg, 5.61 mmol) and N,N-dimethylformamide (7 mL) and the reaction was stirred at 80° C. for 16 hours. The reaction was then diluted with ethyl acetate (25 mL), washed with water (2×10 mL) and brine (10 mL), dried with anhydrous MgSO₄, concentrated and purified by silica gel column chromatography (0-70% ethyl acetate in heptane) to give N-[(1S)-3-chloro-1-methyl-propyl]-1-cyclopropyl-methanesulfonamide (208 mg, 0.77 mmol, 35% yield). LCMS (m/z) ES+268 [M+1]+.

### Step 2

### (3S)-6-cyclohexyl-3-methyl-thiazinane 1,1-dioxide

To a solution of N-[(1S)-3-chloro-1-methyl-propyl]-1-cyclohexyl-methanesulfonamide (208 mg, 0.77 mmol) in tetrahydrofuran (5 mL) was added 1,10-phenanthroline (0.3 mg, 0.002 mmol) and diisopropylamine (0.03 mL, 0.2 mmol) and the solution was cooled to −78° C. N-Butyllithium (2.5 M in hexanes, 0.78 mL, 1.94 mmol) was then added dropwise over 1 minute. The reaction was allowed to warm to room temperature and was stirred at that temperature for 16 hours. The reaction was quenched with saturated NH₄Cl (aq) (5 mL), extracted with dichloromethane (2×10 mL), dried with anhydrous MgSO₄ and concentrated to give (3S)-6-cyclohexyl-3-methyl-thiazinane 1,1-dioxide (200 mg, 0.86 mmol, 111% yield). The product was used without further purification. LCMS (m/z) ES⁺232 [M+1]⁺.

### Step 3

### (3S)-2-[(4-bromo-2,5-difluoro-phenyl)methyl]-6-cyclohexyl-3-methyl-thiazinane 1,1-dioxide

To a solution of (3S)-6-cyclohexyl-3-methyl-thiazinane 1,1-dioxide (200 mg, 0.86 mmol) and 1-bromo-4-(chloromethyl)-2,5-difluoro-benzene (225 mg, 0.93 mmol) in N,N-dimethylacetamide (5 mL) at 0° C. was added sodium hydride (60% in mineral oil, 47 mg, 1.17 mmol) and the reaction was allowed to warm to room temperature and stirred at that temperature for 2 hours. Water (5 mL) was then added and the reaction was diluted with ethyl acetate (20 mL), washed with water (2×10 mL) and brine (5 mL), dried with anhydrous MgSO₄, concentrated and purified by silica gel column chromatography (0-70% ethyl acetate in heptane) to give (3S)-2-[(4-bromo-2,5-difluoro-phenyl)methyl]-6-cyclohexyl-3-methyl-thiazinane 1,1-dioxide (335 mg, 0.77 mmol, 99% yield). LCMS (m/z) ES⁺436 [M+1]⁺.

### Step 4

### (3S)-6-cyclohexyl-2-[[2,5-difluoro-4-[(1S,5R)-6-(1,2,4-triazol-4-yl)-3-azabicyclo[3.1.0]hexan-3-yl]phenyl]methyl]-3-methyl-thiazinane 1,1-dioxide

A vial was charged with (3S)-2-[(4-bromo-2,5-difluoro-phenyl)methyl]-6-cyclohexyl-3-methyl-thiazinane 1,1-dioxide (146 mg, 0.33 mmol), (1S,5R)-6-(1,2,4-triazol-4-yl)-3-azabicyclo[3.1.0]hexane hydrochloride (95 mg, 0.50 mmol), palladium(II) acetate (7.6 mg, 0.033 mmol), 2-dicyclohexylphosphino-2′,6′-di-i-propoxy-1,1′-biphenyl (32 mg, 0.066 mmol) and cesium carbonate (546 mg, 1.67 mmol) and the vial was purged with nitrogen for 2 minutes. 1,4-Dioxane (2 mL) was then added and the reaction was stirred at 100° C. for 16 hours. The mixture was diluted with dichloromethane (2 mL) and filtered through celite. The filtrate was concentrated and purified by reverse-phase preparative HPLC, followed by chiral supercritical fluid chromatography to give to give (3S)-6-cyclohexyl-2-[[2,5-difluoro-4-[(1S,5R)-6-(1,2,4-triazol-4-yl)-3-azabicyclo[3.1.0]hexan-3-yl]phenyl]methyl]-3-methyl-thiazinane 1,1-dioxide (ISOMER A) (5.6 mg, 0.011 mmol, 3.3% yield) and (3S)-6-cyclohexyl-2-[[2,5-difluoro-4-[(1S,5R)-6-(1,2,4-triazol-4-yl)-3-azabicyclo[3.1.0]hexan-3-yl]phenyl]methyl]-3-methyl-thiazinane 1,1-dioxide (ISOMER B) (6.7 mg, 0.013 mmol, 4.0% yield).

(3S)-6-cyclohexyl-2-[[2,5-difluoro-4-[(1S,5R)-6-(1,2,4-triazol-4-yl)-3-azabicyclo[3.1.0]hexan-3-yl]phenyl]methyl]-3-methyl-thiazinane 1,1-dioxide (ISOMER A): ¹H NMR (400 MHz, DMSO) δ 8.62-8.58 (s, 2H), 7.10-7.02 (m, 1H), 6.64-6.55 (m, 1H), 4.92-4.85 (m, 1H), 3.97-3.88 (m, 1H), 3.87-3.78 (m, 2H), 3.54-3.48 (m, 1H), 3.39-3.33 (m, 2H), 3.02-2.90 (m, 2H), 2.40-2.35 (m, 2H), 2.30-2.19 (m, 1H), 2.09-1.98 (m, 1H), 1.91-1.72 (m, 4H), 1.70-1.46 (m, 4H), 1.36-1.29 (d, J=6.3 Hz, 3H), 1.25-1.00 (m, 5H); LCMS (m/z) ES⁺506.2 [M+1]⁺.

(3S)-6-cyclohexyl-2-[[2,5-difluoro-4-[(1S,5R)-6-(1,2,4-triazol-4-yl)-3-azabicyclo[3.1.0]hexan-3-yl]phenyl]methyl]-3-methyl-thiazinane 1,1-dioxide (ISOMER A): ¹H NMR (400 MHz, DMSO) δ 8.61-8.58 (s, 2H), 7.04-6.96 (dd, J=14.4, 7.2 Hz, 1H), 6.61-6.54 (m, 1H), 5.02-4.95 (d, J=8.2 Hz, 1H), 4.21-4.12 (m, 1H), 3.88-3.78 (m, 2H), 3.54-3.48 (t, J=2.2 Hz, 1H), 3.38-3.33 (m, 2H), 3.13-3.07 (dd, J=14.0, 5.1 Hz, 1H), 2.65-2.57 (dd, J=14.0, 7.3 Hz, 1H), 2.40-2.35 (m, 2H), 2.19-2.05 (m, 1H), 1.90-1.82 (d, J=13.2 Hz, 1H), 1.79-1.53 (m, 8H), 1.31-1.26 (d, J=6.3 Hz, 3H), 1.19-0.90 (m, 5H); LCMS (m/z) ES⁺506.2 [M+1]⁺.

The above compounds, together with additional compounds made using the above procedures, are shown in Table 1 below, together with RORc IC₅₀ (micromolar) data for selected compounds. Structures shown in Table 1 with a “waved line” bond ( [symbol] ) associated with a chiral center represent compounds for which stereoisomers of the compound have been isolated or synthesized, but for which specific stereochemistry of the chiral center has not been definitely identified.

TABLE 1

| | | | IC₅₀ |
|---|---|---|---|
| | Structure | IUPAC Name | (μM) |
| 1 | 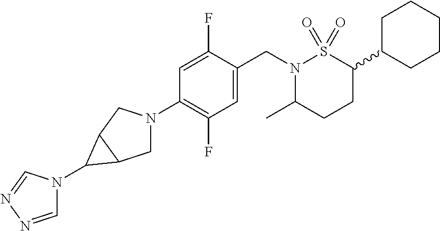 [SMILES: CC1CCC(C2CCCCC2)S(=O)(=O)N1Cc1cc(F)c(N2CC3C(C2)C3n2cnnc2)cc1F] | 3-[4-(6-Cyclohexyl-3- methyl-1,1-dioxo- [1,2]thiazinan-2-ylmethyl)- 2,5-difluoro-phenyl]-6- [1,2,4]triazol-4-yl-3-aza- bicyclo [3.1.0]hexane Diastereomer A | 0.043 |
| 2 | 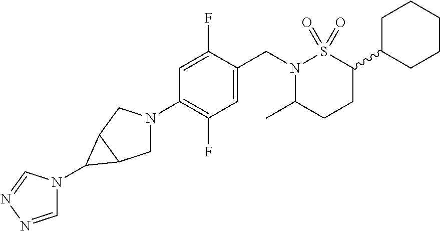 [SMILES: CC1CCC(C2CCCCC2)S(=O)(=O)N1Cc1cc(F)c(N2CC3C(C2)C3n2cnnc2)cc1F] | 3-[4-(6-Cyclohexyl-3- methyl-1,1-dioxo- [1,2]thiazinan-2-ylmethyl)- 2,5-difluoro-phenyl]-6- [1,2,4]triazol-4-yl-3-aza- bicyclo [3.1.0]hexane Diastereomer B | 0.070 |
| 3 | 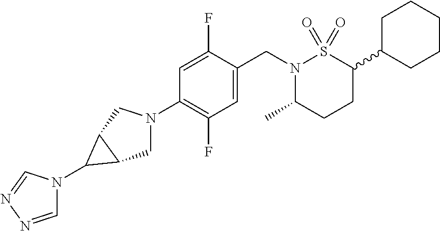 [SMILES: C[C@H]1CCC(C2CCCCC2)S(=O)(=O)N1Cc1cc(F)c(N2C[C@@H]3C(n4cnnc4)[C@@H]3C2)cc1F] | (1S,5R,6S)-3-[4-(6- Cyclopropyl-3-methyl-1,1- dioxo[1,2]thiazinan-2- ylmethyl)-2,5-difluoro- phenyl]-6-[1,2,4]triazol-4- yl-3-aza-bicyclo [3.1.0]hexane Diastereomer A | 0.030 |
| 4 | 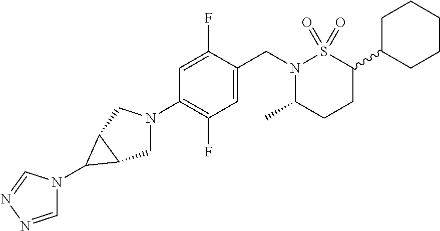 [SMILES: C[C@H]1CCC(C2CCCCC2)S(=O)(=O)N1Cc1cc(F)c(N2C[C@@H]3C(n4cnnc4)[C@@H]3C2)cc1F] | (1S,5R,6S)-3-[4-((6- Cyclopropyl-3-methyl-1,1- dioxo-[1,2]thiazinan-2- ylmethyl)-2,5-difluoro- phenyl]-6-[1,2,4]triazol-4- yl-3-aza-bicyclo [3.1.0]hexane Diastereomer B | 0.249 |
| 5 | 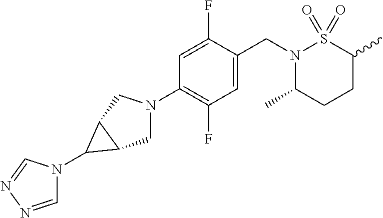 [SMILES: CC1CC[C@H](C)N(Cc2cc(F)c(N3C[C@@H]4C(n5cnnc5)[C@@H]4C3)cc2F)S1(=O)=O] | (1S,5R,6S)-3-[4-(3,6- Dimethyl-1,1-dioxo- 1,2]thiazinan-2-ylmethyl)- 2,5-difluoro-phenyl]-6- [1,2,4]triazol-4-yl-3-aza- bicyclo [3.1.0]hexane Diastereomer A | 0.074 |
| 6 | 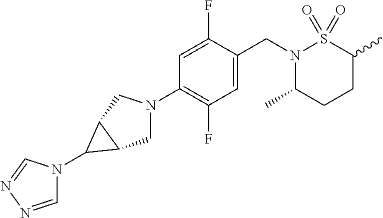 [SMILES: CC1CC[C@H](C)N(Cc2cc(F)c(N3C[C@@H]4C(n5cnnc5)[C@@H]4C3)cc2F)S1(=O)=O] | (1S,5R,6S)-3-[4-(3,6- Dimethyl-1,1-dioxo- [1,2]thiazinan-2-ylmethyl)- 2,5-difluoro-phenyl]-6- [1,2,4]triazol-4-yl-3-aza- bicyclo [3.1.0]hexane Diastereomer B | 0.583 |
| 7 | 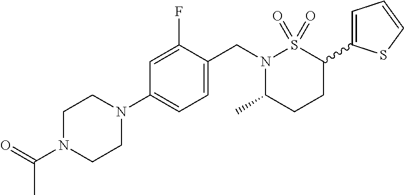 [SMILES: CC(=O)N1CCN(c2ccc(CN3[C@@H](C)CCC(c4cccs4)S3(=O)=O)c(F)c2)CC1] | 1-{4-[3-Fluoro-4-(3-methyl- 1,1-dioxo-6-thiophen-2-yl- [1,2]thiazinan-2-ylmethyl)- phenyl]-piperazin-1-yl}- ethanone | 0.018 |
| 8 | 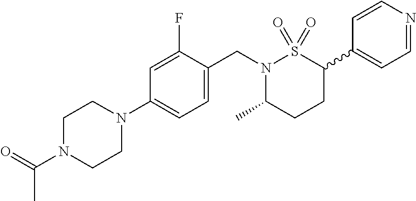 [SMILES: CC(=O)N1CCN(c2ccc(CN3[C@@H](C)CCC(c4ccncc4)S3(=O)=O)c(F)c2)CC1] | 1-{4-[3-Fluoro-4-(3-methyl- 1,1-dioxo-6-pyridin-4-yl- [1,2]thiazinan-2-ylmethyl)- phenyl]-piperazin-1-yl}- ethanone Diastereomer A | 1.000 |
| 9 | 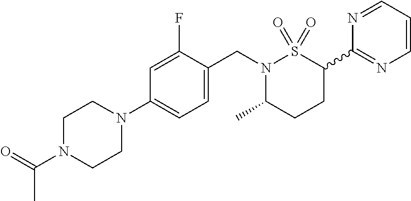 [SMILES: CC(=O)N1CCN(c2ccc(CN3[C@@H](C)CCC(c4ncccn4)S3(=O)=O)c(F)c2)CC1] | 1-{4-[3-Fluoro-4-(3-methyl- 1,1-dioxo-6-pyrimidin-2-yl- [1,2]thiazinan-2-ylmethyl)- phenyl]-piperazin-1-yl}- ethanone Diastereomer A | 0.792 |
| 10 | 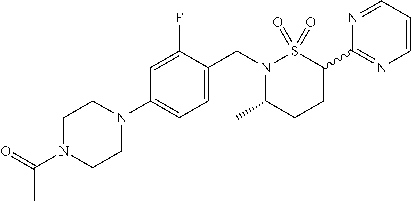 [SMILES: CC(=O)N1CCN(c2ccc(CN3[C@@H](C)CCC(c4ncccn4)S3(=O)=O)c(F)c2)CC1] | 1-{4-[3-Fluoro-4-(3-methyl- 1,1-dioxo-6-pyrimidin-2-yl- [1,2]thiazinan-2-ylmethyl)- phenyl]-piperazin-1-yl}- ethanone Diastereomer B | 1.100 |
| 11 | 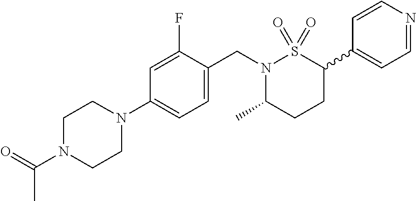 [SMILES: CC(=O)N1CCN(c2ccc(CN3[C@@H](C)CCC(c4ccncc4)S3(=O)=O)c(F)c2)CC1] | 1-{4-[3-Fluoro-4-(3-methyl- 1,1-dioxo-6-pyridin-4-yl- [1,2]thiazinan-2-ylmethyl)- phenyl]-piperazin-1-yl}- ethanone Diastereomer B | 2.200 |
| 12 | 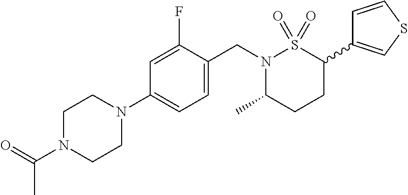 [SMILES: CC(=O)N1CCN(c2ccc(CN3[C@@H](C)CCC(c4ccsc4)S3(=O)=O)c(F)c2)CC1] | 1-{4-[3-Fluoro-4-(3-methyl- 1,1-dioxo-6-thiophen-3-yl- [1,2]thiazinan-2-ylmethyl)- phenyl]-piperazin-1-yl}- ethanone Diastereomer A | 0.036 |
| 13 | 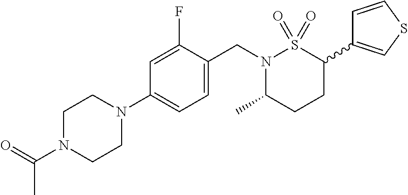 [SMILES: CC(=O)N1CCN(c2ccc(CN3[C@@H](C)CCC(c4ccsc4)S3(=O)=O)c(F)c2)CC1] | 1-{4-[3-Fluoro-4-(3-methyl- 1,1-dioxo-6-thiophen-3-yl- [1,2]thiazinan-2-ylmethyl)- phenyl]-piperazin-1-yl}- ethanone Diastereomer B | 0.123 |
| 14 | 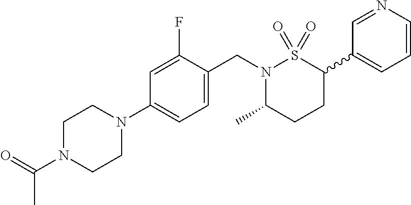 [SMILES: CC(=O)N1CCN(c2ccc(CN3[C@@H](C)CCC(c4cccnc4)S3(=O)=O)c(F)c2)CC1] | 1-{4-[3-Fluoro-4-(3-methyl- 1,1-dioxo-6-pyridin-3-yl- [1,2]thiazinan-2-ylmethyl)- phenyl]-piperazin-1-yl}- ethanone Diastereomer A | 0.870 |
| 15 | 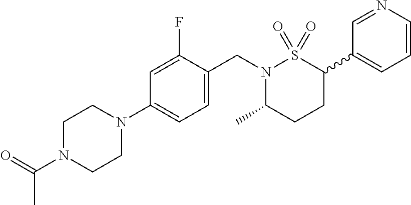 [SMILES: CC(=O)N1CCN(c2ccc(CN3[C@@H](C)CCC(c4cccnc4)S3(=O)=O)c(F)c2)CC1] | 1-{4-[3-Fluoro-4-(3-methyl- 1,1-dioxo-6-pyridin-3-yl- [1,2]thiazinan-2-ylmethyl)- phenyl]-piperazin-1-yl}- ethanone Diastereomer B | 4.200 |
| 16 | 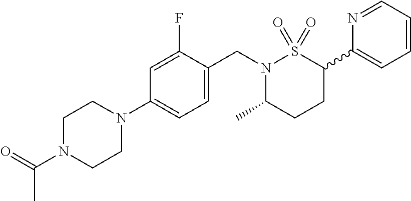 [SMILES: CC(=O)N1CCN(c2ccc(CN3[C@@H](C)CCC(c4ccccn4)S3(=O)=O)c(F)c2)CC1] | 1-{4-[3-Fluoro-4-(3-methyl- 1,1-dioxo-6-pyridin-2-yl- [1,2]thiazinan-2-ylmethyl)- phenyl]-piperazin-1-yl}- ethanone Diastereomer A | 0.292 |
| 17 | 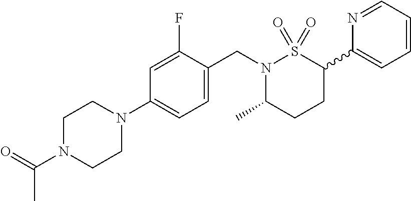 [SMILES: CC(=O)N1CCN(c2ccc(CN3[C@@H](C)CCC(c4ccccn4)S3(=O)=O)c(F)c2)CC1] | 1-{4-[3-Fluoro-4-((3S,6S)-3- methyl-1,1-dioxo-6-pyridin- 2-yl-[1,2]thiazinan-2- ylmethyl)-phenyl]-piperazin- 1-yl}-ethanone Diastereomer B | 0.825 |
| 18 | 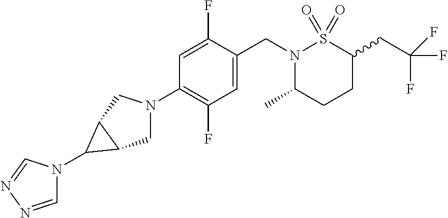 [SMILES: C[C@H]1CCC(CC(F)(F)F)S(=O)(=O)N1Cc1cc(F)c(N2C[C@@H]3C(n4cnnc4)[C@@H]3C2)cc1F] | (3S)-2-(4-((1R,5S)-6-(4H- 1,2,4-triazol-4-yl)-3- azabicyclo [3.1.0]hexan-3- yl)-2,5-difluorobenzyl)-3- methyl-6-(2,2,2- trifluoroethyl)-1,2-thiazinane 1,1-dioxide Diastereomer A | |
| 19 | 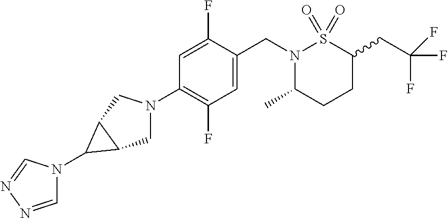 [SMILES: C[C@H]1CCC(CC(F)(F)F)S(=O)(=O)N1Cc1cc(F)c(N2C[C@@H]3C(n4cnnc4)[C@@H]3C2)cc1F] | (3S)-2-(4-((1R,5S)-6-(4H- 1,2,4-triazol-4-yl)-3- azabicyclo [3.1.0]hexan-3- yl)-2,5-difluorobenzyl)-3- methyl-6-(2,2,2- trifluoroethyl)-1,2-thiazinane 1,1-dioxide Diastereomer B | |
| 20 | 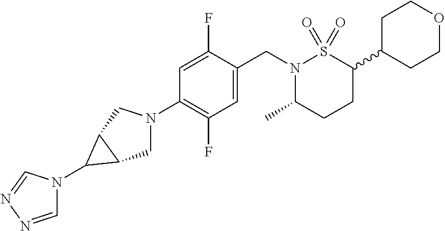 [SMILES: C[C@H]1CCC(C2CCOCC2)S(=O)(=O)N1Cc1cc(F)c(N2C[C@@H]3C(n4cnnc4)[C@@H]3C2)cc1F] | (3S)-2-(4-((1R,5S)-6-(4H- 1,2,4-triazol-4-yl)-3- azabicyclo [3.1.0]hexan-3- yl)-2,5-difluorobenzyl)-3- methyl-6-(tetrahydro-2H- pyran-4-yl)-1,2-thiazinane 1,1-dioxide Diastereomer A | |
| 21 | 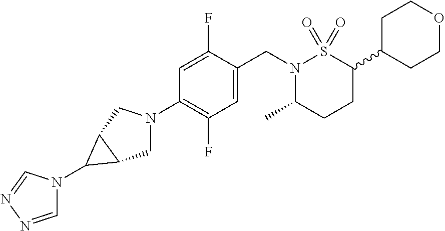 [SMILES: C[C@H]1CCC(C2CCOCC2)S(=O)(=O)N1Cc1cc(F)c(N2C[C@@H]3C(n4cnnc4)[C@@H]3C2)cc1F] | (3S)-2-(4-((1R,5S)-6-(4H- 1,2,4-triazol-4-yl)-3- azabicyclo [3.1.0]hexan-3- yl)-2,5-difluorobenzyl)-3- methyl-6-(tetrahydro-2H- pyran-4-yl)-1,2-thiazinane 1,1-dioxide Diastereomer B | |
| 22 | 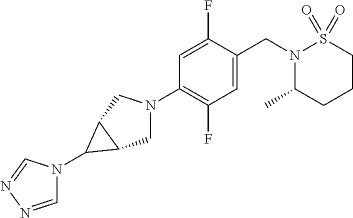 [SMILES: C[C@H]1CCCS(=O)(=O)N1Cc1cc(F)c(N2C[C@@H]3C(n4cnnc4)[C@@H]3C2)cc1F] | (1S,5R,6S)-3-[2,5-Difluoro- 4-((S)-3-methyl-1,1-dioxo- [1,2]thiazinan-2-ylmethyl)- phenyl]-6-[1,2,4]triazol-4- yl-3-aza-bicyclo [3.1.0]hexane | 0.601 |

### Example 5

### In Vitro RORc Ligand Binding Assay

This assay was used to determine a compound's potency in inhibiting activity of RORc by determining, Kiₐₚₚ, IC₅₀, or percent inhibition values. Consumables used in this Example are shown in Table 5 below.

TABLE 2
Table 2

| Consumable | Supplier and product code |
|---|---|
| GFB Unifilter plates | Perkin Elmer 6005177 |
| 3-[(3- | Sigma C5070 |
| Cholamidopropyl)dimethylammonio]- | |
| 1-propanesulfonate (CHAPS) | |
| 96-well polypropylene U-bottom | Nunc 267245 |
| assay plate | |
| HEPES buffer, 1M | Sigma H3375 |
| Magnesium chloride (MgCl₂) | Sigma M8266 |
| D,L-Dithiothreitol (DTT) | Sigma D0632 |
| Sodium chloride (NaCl) | Sigma 71382 |
| Bovine serum albumin (BSA) | Sigma A7030 [lyophilized powder, |
| | ≧98% (agarose gel |
| | electrophoresis), Essentially |
| | fatty acid free, essentially |
| | globulin free] |
| 25-hydroxycholesterol | Sigma H1015 |
| 25-[26,27-³H]hydroxycholesterol | Perkin Elmer NET674250UC |
| | American Radiolabeled |
| | Chemicals ART0766 |
| RORc ligand binding domain | Genentech (e.g., PUR 28048), |
| | expressed in E. coli |
| Plate seals | Perkin Elmer 6005185 |
| Microscint 0 | Perkin Elmer 6013611 |

### Filter Plate Preparation

On day of the assay, 100 uL of 0.05% CHAPS (in deionized H₂O) was added to all wells of the GFB Unifilter plate and allowed soak for 1 h. A wash buffer of 50 mM HEPES (pH 7.4), 150 mM NaCl, and 5 mM MgCl₂ was prepared to wash the filter plate. To prepare an assay buffer, BSA was added to the wash buffer to reach 0.01% and DTT was added to reach 1 mM.

### Compounds

For IC₅₀ mode, 10 mM compound stocks were serially diluted in DMSO with DMSO to give 20× required final concentration in DMSO (15 uL compound+30 uL DMSO). The 20× compound stocks were diluted in DMSO with Assay Buffer 4-fold to reach 5× the final test concentration in 25% DMSO (10 uL compound+30 uL Assay Buffer). Solutions were mixed by aspiration several times with a pipette set on 50 uL volume. For the assay, 10 uL of 5× compound stock solutions in 25% DMSO were added to the assay plate in duplicate.

For two point screening, 10 mM stock compound solutions were diluted in DMSO to obtain 200 uM (20× the high test concentration) and then diluted 10-fold further to reach 20 uM (20× the low test concentration). The 20× stocks were diluted 4-fold with Assay Buffer (10 uL compound+30 uL Assay Buffer) to reach 5× the test concentrations (50 uM and 5 uM) and 10 uL were added to two assay plates for the duplicate wells. With each concentration tested on 2 plates, each set of 80 compounds used 4 assay plates (1 uM and 10 uM, with n=2).

### Nonspecific Binding (NSB) Samples, Total Binding (TB) Samples and No Receptor (No R) Samples

25-hydroxycholesterol (1 uM) was used to determine the level of NSB signal is prepared in DMSO as for compounds above, then diluted in Assay Buffer to give a final concentration of 5 uM. For 25-hydroxycholesterol in 25% DMSO/75% Assay Buffer; 10 uL per well was used for NSB samples. Wells for Total Binding and No Receptor sample determination contained 10 uL of 25% DMSO/75% Assay Buffer per well.

### Radioligand (25-[³H]Hydroxycholesterol) Preparation

25-[³H]hydroxycholesterol was dilute in Assay Buffer to obtain 15 nM and vortex to mix. Add 20 uL to all wells to reach 6 nM final in the assay.

### Receptor Preparation

The optimal concentration for RORc receptor was found to be 0.6 ug/mL. Stock receptor solution was diluted in assay buffer to obtain 1.5 ug/mL in Assay Buffer. 20 uL was added to all wells. For No R samples, 20 uL Assay Buffer was substituted for receptor solution.

### Sample Addition to Plates and Incubation

Assay plates were 96-well polypropylene V-bottom plates. 10 uL of 5× compound in 25% DMSO/75% Assay Buffer was added to Test wells. 10 uL of 25% DMSO/75% Assay Buffer was added to Total Binding or No Receptor wells. 10 uL of 5 uM 25-hydroxycholesterol in 25% DMSO/75% Assay Buffer was added to NSB wells. 20 uL of 15 nM 25-[³H]hydroxycholesterol prepared in Assay Buffer was added to all wells. 20 uL of 1.5 ug/mL RORc receptor was added to wells (or 40 uL Assay Buffer to No R wells). Following addition to the wells, the plates were incubated 3 h at 25° C.

### Filtration

Using a Packard Filtermate Harvester, the filter plate were washed 4 times following transfer of the incubated samples. Plates were dry-filtered completely (2 h at 50° C. or overnight at room temperature). 50 uL Microscint 0 was added to all wells and read on Topcount protocol Inverted.

### Final Concentrations

Final concentrations were as follows: 50 mM HEPES buffer (pH 7.4); 150 mM NaCl; 1 mM DTT; 5 mM MgCl₂, 0.01% BSA; 5% DMSO; 0.6 ug/mL RORc receptor; 6 nM 25-[³H]hydroxycholesterol. For NSB wells, 1 uM 25-hydroxycholesterol was also present.

### Example 10

### Arthritis Mouse Model

8 to 10-week old male DBA/1 (DBA/1OlaHsd, Harlan Laboratories) mice are housed in a specific pathogen free (SPF) animal facility. Arthritis is induced by two injections of collagen subcutaneously in the base of the tail. The initial injection (on day 0) uses bovine type II collagen (2 mg/ml from Chondrex, Redmond, Wash.) emulsified in equal volume of CFA containing 4 mg/ml of M. tuberculosis (Chondrex). The CII booster injection on Day 29 is emulsified in incomplete Freund's adjuvant (IFA). Each animal receives 0.1 ml of emulsion by subcutaneous/intradermal injection in the tail 2 to 3 cm from the body of the mouse. The booster injection site is in the vicinity of but different from the initial injection site and closer to the body of the animal. OR-1050 was formulated in HRC-6 as above. On weekdays, the animals received two doses (a.m. and p.m.) of HRC-6 or 50 mg/kg OR-1050 p.o. (2.5 mls/kg). On weekends, a single dose of 100 mg/kg was administered (5 mls/kg).

The mice were observed daily for clinical symptoms of CIA based on the following qualitative scale. Each paw was examined individually and scored. Grade 0, normal; grade 1, mild but definite redness and swelling of the ankle or wrist, or apparent redness and swelling limited to individual digits, regardless of the number of affected digits; grade 2, moderate redness and swelling of ankle or wrist; grade 3, severe redness and swelling of the entire paw including digits; grade 4, maximally inflamed limb with involvement of multiple joints. To estimate cumulative disease severity for each animal, an area under the curve score was calculated for each animal by totaling the sum of the daily hind paw measurements between days 24 and 48.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

What is claimed is:

1. A compound of formula I 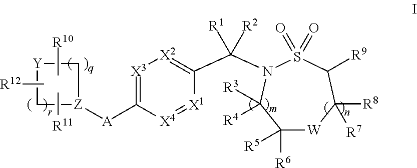
or a pharmaceutically acceptable salt thereof,
wherein:
m is 0 or 1;
n is 0 or 1;
q is 0, 1 or 2;
r is from 1 to 3;
A is: a bond; —(CRⱼRₖ)ₜ—; —C(O)—(CRⱼRₖ)ₜ—; —(CRⱼRₖ)ₜ—C(O)—; —NR^{a}—(CRⱼRₖ)ₜ—; —(CRⱼRₖ)ₜ—NR^{a}—; —C(O)NR^{a}—(CRⱼRₖ)ₜ—; —(CRⱼRₖ)ₜ—NR^{a}C(O)—; —O—(CRⱼRₖ)ₜ—; —(CRⱼRₖ)ₜ—O—; —S—(CRⱼRₖ)ₜ—; —(CRⱼRₖ)ₜ—S—; —SO₂—(CRⱼRₖ)ₜ—; or —(CRⱼRₖ)ₜ—SO₂—;
t is from 0 to 4;
W is: —CR^{b}R^{c}—; —O—; —S—; —SO₂—; or NR^{d}—;
one of X¹, X², X³ and X⁴ is N and the others are CR^{e}; or two of X¹, X², X³ and X⁴ are N and the others are CR^{e}; or three of X¹, X², X³ and X⁴ are N and the other is CR^{e}; or each of X¹, X², X³ and X⁴ is CR^{e};
Y is: —O—; —S—; SO₂—; —CR^{f}R^{g}—; or NR^{h}—;
Z is: CH; or N;
R¹, R², R³, R⁴, R⁵, R⁶, R⁷ and R⁸ each independently is: hydrogen; or C₁₋₆alkyl which may be unsubstituted or substituted one or more times with halo;
or R³ and R⁴ together with the atom to which they are attached may form an ethylene group;
or R³ and R⁴ together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;
or R⁵ and R⁶ together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;
or R⁷ and R⁸ together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;
or one of R³ and R⁴ together with one of R⁵ and R⁶ and the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;
or one of R⁵ and R⁶ together with one of R⁷ and R⁸ and the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;
R⁹ is: C₁₋₆alkyl; C₃-cycloalkyl; heterocyclyl; or heteroaryl; each of which may be unsubstituted or substituted one or more times with Rⁱ;
R¹⁰ is: hydrogen; carboxy; C₁₋₆alkyl-carbonyl; C₁₋₆alkoxy-carbonyl; oxo; hydroxy; aminocarbonyl; N—C₁₋₆alkyl-aminocarbonyl; N,N-di-C₁₋₆alkyl-aminocarbonyl; cyano; hydroxy-C₁₆alkyl; N—C₁₋₆alkoxy-C₁₋₆alkyl-aminocarbonyl; N-hydroxy-C₁₋₆alkyl-aminocarbonyl; N—C₁₋₆alkoxy-aminocarbonyl; halo; or C₁₋₆alkyl which may be unsubstituted or substituted one or more times with halo or oxo;
R¹¹ is: hydrogen; halo; carboxy; C₁₋₆alkyl-carbonyl; C₁₋₆alkoxy-carbonyl; oxo; hydroxy; aminocarbonyl; N—C₁₋₆alkyl-aminocarbonyl; N,N-di-C₁₋₆alkyl-aminocarbonyl; C₁₋₆alkyl-sulfonylamino; C₁₋₆alkyl-sulfonylamino-C₁₋₆alkyl; cyano; hydroxy-C₁₆alkyl; N—C₁₋₆alkoxy-C₁₋₆alkyl-aminocarbonyl; N-hydroxy-C₁₋₆alkyl-aminocarbonyl; N—C₁₋₆alkoxy-aminocarbonyl; or C₁₋₆alkyl which may be unsubstituted or substituted one or more times with halo or oxo;
R¹² is: hydrogen; halo; carboxy; C₁₋₆alkyl-carbonyl; C₁₋₆alkoxy-carbonyl; oxo; hydroxy; aminocarbonyl; N—C₁₋₆alkyl-aminocarbonyl; N,N-di-C₁₋₆alkyl-aminocarbonyl; cyano; hydroxy-C₁₆alkyl; N—C₁₋₆alkoxy-C₁₋₆alkyl-aminocarbonyl; N-hydroxy-C₁₋₆alkyl-aminocarbonyl; N—C₁₋₆alkoxy-aminocarbonyl; or C₁₋₆alkyl which may be unsubstituted or substituted one or more times with halo or oxo;
or R¹⁰ and R¹¹ together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;
R^{a}, R^{b}, and R^{d} each independent is: hydrogen; or C₁₋₆alkyl which may be unsubstituted or substituted one or more times with halo;
or R^{b} and R^{c} together with the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;
or one of R^{b} and R^{c} together with one of R⁷ and R⁸ and the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;
or one of R^{b} and R^{c} together with one of R⁵ and R⁶ and the atoms to which they are attached may form a three, four, five, six or seven membered saturated or partially saturated ring that may optionally include one or two heteroatoms selected from —O—, —NR^{a}— or —S—, and which may be optionally substituted one or more times with Rⁱ;
each R^{e} is independently: hydrogen; C₁₋₆alkyl; halo; C₁₋₆alkoxy; or cyano; wherein the C₁₋₆alkyl moieties may be unsubstituted or substituted one or more times with halo;
R^{f} is: hydrogen; halo; or C₁₋₆alkyl which may be unsubstituted or substituted one or more times with halo;
R^{g} is: hydrogen; C₁₋₆alkyl; C₃₋₆cycloalkyl; C₃₋₆cycloalkenyl; C₃₋₆cycloalkyl-C₁₋₆alkyl; halo; C₁₋₆alkyl-carbonyl; C₃₋₆cycloalkyl-carbonyl; C₃₋₆cycloalkyl-C₁₋₆alkyl-carbonyl; cyano-C₁₋₆alkyl-carbonyl; hydroxy-C₁₋₆alkyl-carbonyl; C₁₋₆alkoxy-C₁₋₆alkyl-carbonyl; carboxy; N-cyano-aminocarbonyl; N-cyano-N—C₁₋₆alkyl-aminocarbonyl; N—C₁₋₆alkyl-acetimidamidyl; N,N′-di-C₁₋₆alkyl-acetimidamidyl; N′-cyano-N—C₁₋₆alkyl-acetimidamidyl; N′-hydroxy-acetimidamidyl; N′—C₁₋₆alkoxy-acetimidamidyl; N′-hydroxy-N—C₁₋₆alkyl-acetimidamidyl; N′—C₁₋₆alkoxy-N—C₁₋₆alkyl-acetimidamidyl; 2-nitro-1-N—C₁₋₆alkylamino-vinyl; formyl; C₁₋₆alkyl-sulfonyl; C₃₋₆cycloalkyl-sulfonyl; C₃₋₆cycloalkyl-C₁₋₆alkyl-sulfonyl; C₁₋₆alkyl-sulfonyl-C₁₋₆alkyl; aminocarbonyl; N-hydroxy-aminocarbonyl; N—C₁₋₆alkoxy-aminocarbonyl; N—C₁₋₆alkyl-aminocarbonyl; aminocarbonyl-C₁₋₆alkyl; N—C₁₋₆alkyl-aminocarbonyl-C₁₋₆alkyl; N,N-di-C₁₋₆alkyl-aminocarbonyl-C₁₋₆alkyl; C₁₋₆alkoxy-carbonyl; N-hydroxy-N—C₁₋₆alkyl-aminocarbonyl; N—C₁₋₆alkoxy-N—C₁₋₆alkyl-aminocarbonyl; N,N-di-C₁₋₆alkyl-aminocarbonyl; aminosulfonyl; N—C₁₋₆alkyl-aminosulfonyl; N,N-di-C₁₋₆alkyl-aminosulfonyl; cyano; C₁₋₆alkoxy; C₁₋₆alkyl-sulfonylamino; N—C₁₋₆alkyl-sulfonylaminocarbonyl; N—(C₁₋₆alkyl-sulfonyl)-N—C₁₋₆alkyl-aminocarbonyl; N—(C₁₋₆alkyl-sulfonyl)-amino-C₁₋₆alkyl; amino; N—C₁₋₆alkyl-amino; N,N-di-C₁₋₆alkyl-amino; halo-C₁₋₆alkyl; heterocyclyl; heteroaryl; or hydroxyl; wherein the C₁₋₆alkyl moieties may be unsubstituted or substituted one or more times with halo; and wherein the heterocyclyl, heteroaryl, C₃₋₆cycloalkyl, C₃₋₆cycloalkenyl and C₃₋₆cycloalkyl-C₁₋₆alkyl moieties may be unsubstituted or substituted one or more times with Rⁱ;
or R^{f} and R^{g} together with the atoms to which they are attached may form a four, five, six or seven membered saturated or partially saturated ring that may optionally include one or two heteroatoms selected from —O—, —NR^{a}— or —S—, and which may be optionally substituted one or more times with Rⁱ;
R^{h} is: hydrogen; C₁₋₆alkyl; C₃₋₆cycloalkyl; C₃₋₆cycloalkenyl; C₃₋₆cycloalkyl-C₁₋₆alkyl; C₁₋₆alkyl-carbonyl; C₃₋₆cycloalkyl-carbonyl; C₃₋₆cycloalkyl-C₁₋₆alkyl-carbonyl; cyano-C₁₋₆alkyl-carbonyl; hydroxy-C₁₋₆alkyl-carbonyl; C₁₋₆alkoxy-C₁₋₆alkyl-carbonyl; N-cyano-aminocarbonyl; N-cyano-N—C₁₋₆alkyl-aminocarbonyl; N—C₁₋₆alkyl-acetimidamidyl; N,N′-di-C₁₋₆alkyl-acetimidamidyl; N′-cyano-N—C₁₋₆alkyl-acetimidamidyl; N′-hydroxy-acetimidamidyl; N′—C₁₋₆alkoxy-acetimidamidyl; N′-hydroxy-N—C₁₋₆alkyl-acetimidamidyl; N′—C₁₋₆alkoxy-N—C₁₋₆alkyl-acetimidamidyl; 2-nitro-1-N—C₁₋₆alkylamino-vinyl; formyl; C₁₋₆alkyl-sulfonyl; C₃₋₆cycloalkyl-sulfonyl; C₃₋₆cycloalkyl-C₁₋₆alkyl-sulfonyl; C₁₋₆alkyl-sulfonyl-C₁₋₆alkyl; aminocarbonyl; N-hydroxy-aminocarbonyl; N—C₁₋₆alkoxy-aminocarbonyl; N—C₁₋₆alkyl-aminocarbonyl; N-hydroxy-N—C₁₋₆alkyl-aminocarbonyl; N—C₁₋₆alkoxy-N—C₁₋₆alkyl-aminocarbonyl; N,N-di-C₁₋₆alkyl-aminocarbonyl; aminosulfonyl; N—C₁₋₆alkyl-aminosulfonyl; N,N-di-C₁₋₆alkyl-aminosulfonyl; cyano; C₁₋₆alkyl-sulfonylamino; C₁₋₆alkyl-sulfonylamino-C₁₋₆alkyl; N—(C₁₋₆alkyl-sulfonyl)aminocarbonyl; N—(C₁₋₆alkyl-sulfonyl)-N—C₁₋₆alkyl-aminocarbonyl; N—(C₁₋₆alkyl-sulfonyl)-amino-C₁₋₆alkyl; aminocarbonyl-C₁₋₆alkyl; N—C₁₋₆alkyl-aminocarbonyl-C₁₋₆alkyl; N,N-di-C₁₋₆alkyl-aminocarbonyl-C₁₋₆alkyl; C₁₋₆alkoxy-carbonyl; halo-C₁₋₆alkyl; heterocyclyl; or heteroaryl; wherein the C₁₋₆alkyl moieties may be unsubstituted or substituted one or more times with halo; and wherein the heterocyclyl, heteroaryl, C₃₋₆cycloalkyl, C₃₋₆cycloalkenyl and C₃₋₆cycloalkyl-C₁₋₆alkyl moieties may be unsubstituted or substituted one or more times with Rⁱ;
or R^{h} and one of R¹⁰ and R¹¹ together with the atoms to which they are attached may form a four, five, six or seven membered aromatic, partially saturated or unsaturated ring that may optionally include one or two additional heteroatom selected from —O—, —NR^{a}— or —S—, and which may be optionally substituted one or more times with Rⁱ—;
or one of R^{f} and R^{g} and one of R¹⁰ and R¹¹ together with the atoms to which they are attached may form a three, four, five, six or seven membered aromatic, partially saturated or unsaturated ring that may optionally include an additional heteroatom selected from —O—, —NR^{a}— or —S—, and which may be optionally substituted one or more times with Rⁱ;
Rⁱ is: C₁₋₆alkyl; halo; oxo; hydroxy; acetyl; C₁₋₆alkyl-carbonyl; amino-carbonyl; hydroxy-C₁₋₆alkyl; cyano; heteroaryl; or C₁₋₆alkoxy; wherein the C₁₋₆alkyl moieties may be unsubstituted or substituted one or more times with halo; and
R^{j} and R^{k} each independent is: hydrogen; or C₁₋₆alkyl which may be unsubstituted or substituted one or more times with halo.

2. The Compound of claim 1, wherein m is 1.

3. The Compound of claim 1, wherein n is 0.

4. The Compound of claim 1, wherein A is a bond; —NR^{a}—; or —O—.

5. The Compound of claim 1, wherein A is a bond.

6. The Compound of claim 1, wherein m is W is —CH₂—.

7. The Compound of claim 1, wherein X¹, X², X³ and X⁴ are CR^{e}.

8. The Compound of claim 1, wherein R¹, R², R⁴, R⁵, R⁶, R⁷ and R⁸ are hydrogen.

9. The Compound of claim 1, wherein R⁹ is C₁₋₆alkyl which may be unsubstituted or substituted one or more times with Rⁱ.

10. The Compound of claim 1, wherein R⁹ is C₃-cycloalkyl which may be unsubstituted or substituted one or more times with Rⁱ.

11. The Compound of claim 1, wherein R⁹ is heterocyclyl selected from azetidinyl, pyrrolidinyl, piperidinyl, azepinyl, oxetanyl, tetrahydrofuranyl and tetrahydropyranyl, each of which may be unsubstituted or substituted one or more times with Rⁱ.

12. The Compound of claim 1, wherein R⁹ is heteroaryl selected from thienyl, furanyl, pyrrolyl, pyrazolyl, oxazolyl, thiazolyl, isoxazolyl, isothiazolyl, triazolyl, pyridinyl, or pyrimidinyl, each of which may be unsubstituted or substituted one or more times with Rⁱ.

13. The Compound of claim 1, wherein R⁹ is thienyl, pyridinyl, or pyrimidinyl.

14. The Compound of claim 1, wherein the compound may is of formula XVIIIa or XVIIIb: 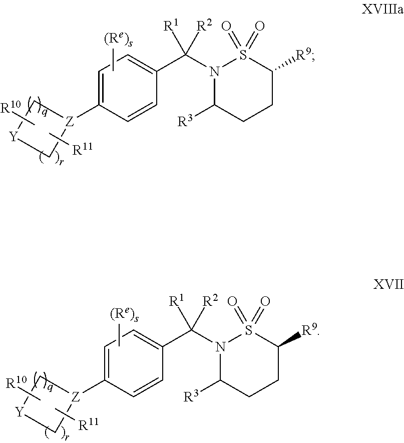
and s is from 0 to 2.

15. The Compound of claim 14, wherein R^{e} is halo.

16. The Compound of claim 15, wherein s is 1.

17. The Compound of claim 15, wherein s is 2.

18. The Compound of claim 15, wherein the group 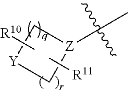
is:

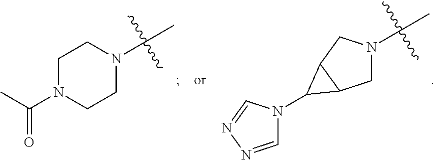

19. A composition comprising:
(a) a pharmaceutically acceptable carrier; and
(b) a compound of claim 1.

20. A method for treating arthritis, said method comprising administering to a subject in need thereof an effective amount of a compound of claim 1.
